SEQUENCE LISTING (42 sequences)

SEQ ID NO: 1 - DNA, 1894 bp, Homo sapiens
cgccgctcgccgcagacttacttccccggctcagcagggaaaggttcctagaaggtgagcgcggacggtatgcaaagttgtgaatccagtggtgacagtgcggatgaccctctcagtcgcggcctacggagaaggggacagcctcgtgtggtggtgatcggcgccggcttggctggcctggctgcagccaaagcacttcttgagcagggtttcacggatgtcactgtgcttgaggcttccagccacatcggaggccgtgtgcagagtgtgaaacttggacacgccacctttgagctgggagccacctggatccatggctcccatgggaaccctatctatcatctagcagaagccaacggcctcctggaagagacaaccgatggggaacgcagcgtgggccgcatcagcctctattccaagaatggcgtggcctgctaccttaccaaccacggccgcaggatccccaaggacgtggttgaggaattcagcgatttatacaacgaggtctataacttgacccaggagttcttccggcacgataaaccagtcaatgctgaaagtcaaaatagcgtgggggtgttcacccgagaggaggtgcgtaaccgcatcaggaatgaccctgacgacccagaggctaccaagcgcctgaagctcgccatgatccagcagtacctgaaggtggagagctgtgagagcagctcacacagcatggacgaggtgtccctgagcgccttcggggagtggaccgagatccccggcgctcaccacatcatcccctcgggcttcatgcgggttgtggagctgctggcggagggcatccctgcccacgtcatccagctagggaaacctgtccgctgcattcactgggaccaggcctcagcccgccccagaggccctgagattgagccccggggtgagggcgaccacaatcacgacactggggagggtggccagggtggagaggagccccgggggggcaggtgggatgaggatgagcagtggtcggtggtggtggagtgcgaggaccgtgagctgatcccggcggaccatgtgattgtgaccgtgtcgctaggtgtgctaaagaggcagtacaccagtttcttccggccaggcctgcccacagagaaggtggctgccatccaccgcctgggcattggcaccaccgacaagatctttctggaattcgaggagcccttctggggccctgagtgcaacagcctacagtttgtgtgggaggacgaagcggagagccacaccctcacctacccacctgagctctggtaccgcaagatctgcggctttgatgtcctctacccgcctgagcgctacggccatgtgctgagcggctggatctgcggggaggaggccctcgtcatggagaagtgtgatgacgaggcagtggccgagatctgcacggagatgctgcgtcagttcacagggaaccccaacattccaaaacctcggcgaatcttgcgctcggcctggggcagcaacccttacttccgtggctcctattcatacacgcaggtgggctccagcggggcggatgtggagaagctggccaagcccctgccgtacacggagagctcaaagacagcgcccatgcaggtgctgttttccggtgaggccacccaccgcaagtactattccaccacccacggtgctctgctgtccggccagcgtgaggctgcccgcctcattgagatgtaccgagacctcttccagcaggggacctgagggctgtcctcgctgctgagaagagccactaactcgtgacctccagcctgccccttgctgccgtgtgctcctgccttcctgatcctctgtagaaaggatttttatcttctgtagagctagccgccctgactgccttcagacctggccctgtagcttt

SEQ ID NO: 2 - protein, 555 aa, Homo sapiens [sequence omitted]

SEQ ID NO: 3 - DNA, 1735 bp, Homo sapiens
cgccgctcgccgcagacttacttccccggctcagcagggaaaggttcctagaaggtgagcgcggacggtatgcaaagttgtgaatccagtggtgacagtgcggatgaccctctcagtcgcggcctacggagaaggggacagcctcgtgtggtggtgatcggcgccggcttggctggcctggctgcagccaaagcacttcttgagcagggtttcacggatgtcactgtgcttgaggcttccagccacgtcggaggccgtgtgcagagtgtgaaacttggacacgccacctttgagccgggagccacctggatccatggctcccatgggaaccctatctatcatctagcagaagccaacggcctcctggaagagacaaccgatggggaacgcagcgtgggccgcatcagcctctattccaagaatggcgtggcctgctaccttaccaaccacggccgcaggatccccaaggacgtggttgaggaattcagcgatttatacaacgaggtctataacttgacccaggagttcttccggcacgataaaccagtcaatgctgaaagtcaaaatagcgtgggggtgttcacccgagaggaggtgcgtaaccgcatcaggaatgaccctgacgacccagaggctaccaagcgcctgaagctcgccatgatccagcagtacctgaaggtggagagctgtgagagcagctcacacagcatggacgaggtgtccctgagcgccttcggggagtggaccgagatccccggcgctcaccacatcatcccctcgggcttcatgcgggttgtggagctgctggcggagggcatccctgcccacgtcatccagctagggaaacctgtccgctgcattcactgggaccaggcctcagcccgccccagaggccctgagattgagccccggggtgtgctaaagaggcagtacaccagtttcttccggccaggcctgcccacagagaaggtggctgccatccaccgcctgggcattggcaccaccgacaagatctttctggaattcgaggagcccttctggggccctgagtgcaacagcctacagtttgtgtgggaggacgaagcggagagccacaccctcacctacccacctgagctctggtaccgcaagatctgcggctttgatgtcctctacccgcctgagcgctacggccatgtgctgagcggctggatctgcggggaggaggccctcgtcatggagaggtgtgatgacgaggcagtggccgagatctgcacggagatgctgcgtcagttcacagggaaccccaacattccaaaacctcggcgaatcttgcgctcggcctggggcagcaacccttacttccgcggctcctattcatacacgcaggtgggctccagcggggcggatgtggagaagctggccaagcccctgccgtacacagagagctcaaagacagcgcccatgcaggtgctgttttccggtgaggccacccaccgcaagtactattccaccacccacggtgctctgctgtccggccagcgtgaggctgcccgcctcattgagatgtaccgagacctcttccagcaggggacctgagggctgtcctcgctgctgagaagagccactaactcgtgacctccagcctgccccttgctgccgtgtgctcctgccttcctgatcctctgtagaaaggatttttatcttctgtagagctagccgccctgactgccttcagacctggccctgtagcttt

SEQ ID NO: 4 - protein, 502 aa, Homo sapiens
MQSCESSGDSADDPLSRGLRRRGQPRVVVIGAGLAGLAAAKALLEQGFTDVTVLEASSHVGGRVQSVKLGHATFEPGATWIHGSHGNPIYHLAEANGLLEETTDGERSVGRISLYSKNGVACYLTNHGRRIPKDVVEEFSDLYNEVYNLTQEFFRHDKPVNAESQNSVGVFTREEVRNRIRNDPDDPEATKRLKLAMIQQYLKVESCESSSHSMDEVSLSAFGEWTEIPGAHHIIPSGFMRVVELLAEGIPAHVIQLGKPVRCIHWDQASARPRGPEIEPRGVLKRQYTSFFRPGLPTEKVAAIHRLGIGTTDKIFLEFEEPFWGPECNSLQFVWEDEAESHTLTYPPELWYRKICGFDVLYPPERYGHVLSGWICGEEALVMERCDDEAVAEICTEMLRQFTGNPNIPKPRRILRSAWGSNPYFRGSYSYTQVGSSGADVEKLAKPLPYTESSKTAPMQVLFSGEATHRKYYSTTHGALLSGQREAARLIEMYRDLFQQGT

SEQ ID NO: 5 - DNA, 799 bp, Homo sapiens
cgccgctcgccgcagacttacttccccggctcagcagggaaaggttcctagaaggtgagcgcggacggtatgcaaagttgtgaatccagtggtgacagtgcggatgaccctctcagtcgcggcctacggagaaggggacagcctcgtgtggtggtgatcggcgccggcttggctggcctggctgcagccaaagcacttcttgagcagggtttcacggatgtcactgtgcttgaggcttccagccacgtcggaggccgtgtgcagagtgtgaaacttggacacgccacctttgagctgggagccacctggatccatggctcccatgggaaccctatctatcatctagcagaagccaacggcctcctggaagagacaaccgatggggaacgcagcgtgggccgcatcagcctctattccaagaatggcgtggcctgctaccttaccaaccacggccgcaggatccccaaggacgtggttgaggaattcagcgatttatacaacgagcccatgcaggtgctgttttccggtgaggccacccaccgcaagtactattccaccacccacggtgctctgctgtccggccagcgtgaggctgcccgcctcattgagatgtaccgagacctcttccagcaggggacctgagggctgtcctcgctgctgagaagagccactaactcgtgacctccagcctgccccttgctgccgtgtgctcctgccttcctgatcctctgtagaaaggatttttatcttctgtagagccagccgccctgactgccttcagacctggccctgtagcttt

SEQ ID NO: 6 - protein, 190 aa, Homo sapiens [sequence omitted]

SEQ ID NO: 7 - DNA, 1825 bp, Homo sapiens
cgccgctcgccgcagacttacttccccggctcagcagggaaaggttcctagaaggtgagcgcggacggtatgcaaagttgtgaatccagtggtgacagtgcggatgaccctctcagtcgcggcctacggagaaggggacagcctcgtgtggtggtgatcggcgccggcttggctggcctggctgcagccaaagcacttcttgagcagggtttcacggatgtcactgtgcttgaggcttccagccacatcggaggccgtgtgcagagtgtgaaacttggacacgccacctttgagctgggagccacctggatccatggctcccatgggaaccctatctatcatctagcagaagccaacggcctcctggaagagacaaccgatggggaacgcagcgtgggccgcatcagcctctattccaagaatggcgtggcctgctaccttaccaaccacggccgcaggatccccaaggacgtggttgaggaattcagcgatttatacaacgaggtctataacttgacccaggagttcttccggcacgataaaccagtcaatgctgaaagtcaaaatagcgtgggggtgttcacccgagaggaggtgcgtaaccgcatcaggaatgaccctgacgacccagaggctaccaagcgcctgaagctcgccatgatccagcagtacctgaaggtggagagctgtgagagcagctcacacagcatggacgaggtgtccctgagcgccttcggggagtggaccgagatccccggcgctcaccacatcatcccctcgggcttcatgcgggttgtggagctgctggcggagggcatccctgcccacgtcatccagctagggaaacctgtccgctgcattcactgggaccaggcctcagcccgccccagaggccctgagattgagccccggggtgtgctaaagaggcagtacaccagtttcttccggccaggcctgcccacagagaaggtggctgccatccaccgcctgggcattggcaccaccgacaagatctttctggaattagaggagcccttctggggccctgagtgcaacagcctacagtttgtgtgggaggacgaagcggagagccacaccctcacctacccacctgagctctggtaccgcaagatctgcggctttgatgtcctctacccgcctgagcgctacggccatgtgctgagcggctggatctgcgggggggaggccctcgtcatggagaagtgtgatgacgaggcagtggccgagatctgcacggagatgctgcgtcagttcacagggaaccccaacattccaaaacctcggcgaatcttgcgctcggcctggggcagcaacccttacttccgcggctcctattcatacacgcaggtgggctccagcggggcggatgtggagaagctggccaagcccctgccgtacacagagagctcaaagacagcgcatggaagctccacaaagcagcagcctggtcaccttttctcttccaagtgcccagaacagcccctggatgctaacaggggcgccgtaaagcccatgcaggtgctgttttccggtgaggccacccaccgcaagtactattccaccacccacggtgctctgctgtccggccagcgtgaggctgcccgcctcattgagatgtaccgagacctcttccagcaggggacctgagggctgtcctcgctgctgagaagagccactaactcgtgacctccagcctgccccttgctgccgtgtgctcctgccttcctgatcctctgtagaaaggatttttatcttctgtagagctagccgccctgactgccttcagacctggccctgtagcttt

SEQ ID NO: 8 - protein, 532 aa, Homo sapiens [sequence omitted]

SEQ ID NO: 9 - DNA, 1073 bp, Homo sapiens
cgccgctcgccgcagacttacttccccggctcagcagggaaaggttcctagaaggtgagcgcggacggtatgcaaagttgtgaatccagtggtgacagtgcggatgaccctctcagtcgcggcctacggagaaggggacagcctcgtgtggtggtgatcggcgccggcttggctggcctggctgcagccaaagcacttcttgagcagggtttcacggatgtcactgtgcttgaggcttccagccacgtcggaggccgtgtgcagagtgtgaaacttggacacgccacctttgagctgggagccacctggatccatggctcccatgggaaccctatctatcatctagcagaagccaacggcctcctggaagagacaaccgatggggaacgcagcgtgggccgcatcagcctctattccaagaatggcgtggcctgctaccttaccaaccacggccgcaggatccccaaggacgtggttgaggaattcagcgatttatacaacgaggtctataacttgacccaggagttcttccggcacgataaaccagtcaatgctgaaagtcaaaatagcgtgggggtgttcacccgagaggaggtgcgtaaccgcatcaggaatgaccctgacgacccagaggctaccaagcgcctgaagctcgccatgatccagcagtacctgaaggtggagagctgtgagagcagctcacacagcatggacgaggtgtccctgagcgccttcggggagtggaccgagatccccggcgctcaccacatcatcccctcgggcttcatgcgggttgcggagctgctggcggagggcatccctgcccacgtcatccagctagggaaacctgtccgctgcattcactgggaccaggcctcagcccgccccagaggccctgagattgagccccggggtgagggcgaccacaatcacgacaccggggagggtggccagggtggagaggagcccctagctgccgtgtgctcctgccttcctgatcctctgtagaaaggatttttatcttctgtagagctagccgccctgactgccttcagacctggccctgtagcttt

SEQ ID NO: 10 - protein, 312 aa, Homo sapiens
MQSCESSGDSADDPLSRGLRRRGQPRVVVIGAGLAGLAAAKALLEQGFTDVTVLEASSHVGGRVQSVKLGHATFELGATWIHGSHGNPIYHLAEANGLLEETTDGERSVGRISLYSKNGVACYLTNHGRRIPKDVVEEFSDLYNEVYNLTQEFFRHDKPVNAESQNSVGVFTREEVRNRIRNDPDDPEATKRLKLAMIQQYLKVESCESSSHSMDEVSLSAFGEWTEIPGAHHIIPSGFMRVAELLAEGIPAHVIQLGKPVRCIHWDQASARPRGPEIEPRGEGDHNHDTGEGGQGGEEPLAAVCSCLPDPL

SEQ ID NO: 11 - DNA, 1171 bp, Homo sapiens
cgccgctcgccgcagacttacttccccggctcagcagggaaaggttcctagaaggtgagcgcggacggtatgcaaagttgtgaatccagtggtgacagtgcggatgaccctctcagtcgcggcctacggagaaggggacagcctcgtgtggtggtgatcggcgccggcttggctggcctggctgcagccaaagcacttcttgagcagggtttcacggatgtcactgtgcttgaggcttccagccacgtcggaggccgtgtgcagagtgtgaaacttggacacgccacctttgagctgggagccacctggatccatggctcccatgggaaccctatctatcatctagcagaagccaacggcctcctggaagagacaaccgatggggaacgcagcgtgggccgcatcagcctctattccaagaatggcgtggcctgctaccttaccaaccacggccgcaggatccccaaggacgtggttgaggaattcagcgatttatacaacgaggtctataacttgacccaggagttcttccggcacgataaaccagtcaatgctgaaagtcaaaatagcgtgggggtgttcacccgagaggaggtgcgtaaccgcatcaggaatgaccctgacgacccagaggccaccaagcgcctgaagctcgccatgatccagcagtacctgaaggtggagagctgtgagagcagctcacacagcatggacgaggtgtccctgagcgccttcggggagtggaccgagatccccggcgctcaccacatcatcccctcgggcttcatgcgggttgtggagctgctggcggagggcatccctgcccacgtcatccagctagggaaacctgtccgctgcattcactgggaccaggcctcagcccgccccagaggccctgagattgagccccggggtgagggcgaccacaatcacgacactggggagggtggccagggtggtgaggctgcccgcctcattgagatgtaccgagacctcttccagcaggggacctgagggctgtcctcgctgctgagaagagccactaactcgtgacctccagcctgccccttgctgccgtgtgctcctgccttcctgatcctctgtagaaaggatttttatcttctgtagagctagccgccctgactgccttcagacctggccctgtagcttt

SEQ ID NO: 12 - protein, 314 aa, Homo sapiens
MQSCESSGDSADDPLSRGLRRRGQPRVVVIGAGLAGLAAAKALLEQGFTDVTVLEASSHVGGRVQSVKLGHATFELGATWIHGSHGNPIYHLAEANGLLEETTDGERSVGRISLYSKNGVACYLTNHGRRIPKDVVEEFSDLYNEVYNLTQEFFRHDKPVNAESQNSVGVFTREEVRNRIRNDPDDPEATKRLKLAMIQQYLKVESCESSSHSMDEVSLSAFGEWTEIPGAHHIIPSGFMRVVELLAEGIPAHVIQLGKPVRCIHWDQASARPRGPEIEPRGEGDHNHDTGEGGQGGEAARLIEMYRDLFQQGT

SEQ ID NO: 13 - DNA, 943 bp, Homo sapiens
cgccgctcgccgcagacttacttccccggctcagcagggaaaggttcctagaaggtgagcgcggacggtatgcaaagttgtgaatccagtggtgacagtgcggatgaccctctcagtcgcggcctacggagaaggggacagcctcgtgtggtggtgatcggcgccggcttggctggcctggctgccatccaccgcctgggcattggcaccaccgacaagatctttctggaattcgaggagcccttctggggccctgagtgcaacagcctacagtttgtgtgggaggacgaagcggagagccacaccctcacctacccacctgagctctggtaccgcaagatctgcggctttgatgtcctctacccgcctgagcgctacggccatgtgctgagcggctggatctgcggggaggaggccctcgtcatggagaagtgtgatgacgaggcagtggccgagatctgcacggagatgctgcgtcagttcacagggaaccccaacattccaaaacctcggcgaatcttgcgctcggcctggggcagcaacccttacttccgcggctcctattcatacacgcaggtgggctccagcggggcggatgtggagaagctggccaagcccctgccgtacacagagagctcaaagacagcgcccatgcgggtgctgttttccggtgaggccacccaccgcaagtactattccaccacccacggtgctctgctgtccggccagcgtgaggctgcccgcctcattgagatgtaccgagacctcttccagcaggggacctgagggctgtcctcgctgctgagaagagccactaactcgtgacctccagcctgccccttgctgccgtgtgctcctgccttcctgatcctctgtagaaaggatttttatcttctgtagagccagccgccctgactgccttcagacctggccctgtagcttt

SEQ ID NO: 14 - protein, 238 aa, Homo sapiens
MQSCESSGDSADDPLSRGLRRRGQPRVVVIGAGLAGLAAIHRLGIGTTDKIFLEFEEPFWGPECNSLQFVWEDEAESHTLTYPPELWYRKICGFDVLYPPERYGHVLSGWICGEEALVMEKCDDEAVAEICTEMLRQFTGNPNIPKPRRILRSAWGSNPYFRGSYSYTQVGSSGADVEKLAKPLPYTESSKTAPMRVLFSGEATHRKYYSTTHGALLSGQREAARLIEMYRDLFQQGT

SEQ ID NO: 15 - DNA, 451 bp, Homo sapiens
cgccgctcgccgcagacttacttccccggctcagcagggaaaggttcctagaaggtgagcgcggacggtatgcaaagttgtgaatccagtggtgacagtgcggatgtggagaagctggccaagcccctgccgtacacggagagctcaaagacagcgcccatgcaggtgctgttttccggtgaggccacccaccgcaagtactattccaccacccacggtgctctgctgtccggccagcgtgaggctgcccgcctcattgagatgtaccgagacctcttccagcaggggacctgagggctgtcctcgctgctgagaagagccactaactcgtgacctccagcctgccccttgctgccgtgtgctcctgccttcctgatcctctgtagaaaggatttttatcttctgtagagccagccgccctgactgccttcagacctggccctgtagcttt

SEQ ID NO: 16 - protein, 74 aa, Homo sapiens
MQSCESSGDSADVEKLAKPLPYTESSKTAPMQVLFSGEATHRKYYSTTHGALLSGQREAARLIEMYRDLFQQGT

SEQ ID NO: 17 - DNA, 12 bp, Homo sapiens
ggaaaggtacgg

SEQ ID NO: 18 - DNA, 12 bp, Homo sapiens
ctgcaggttcct

SEQ ID NO: 19 - DNA, 12 bp, Homo sapiens
aacttggtaagt

SEQ ID NO: 20 - DNA, 12 bp, Homo sapiens
cctcaggacacg

SEQ ID NO: 21 - DNA, 12 bp, Homo sapiens
aacgaggtaagg

SEQ ID NO: 22 - DNA, 12 bp, Homo sapiens
tggcaggtctat

SEQ ID NO: 23 - DNA, 12 bp, Homo sapiens
ctgaaggtatct

SEQ ID NO: 24 - DNA, 12 bp, Homo sapiens
ccgcaggtggag

SEQ ID NO: 25 - DNA, 12 bp, Homo sapiens
tcacaggtgcgc

SEQ ID NO: 26 - DNA, 12 bp, Homo sapiens
catcagggaacc

SEQ ID NO: 27 - DNA, 12 bp, Homo sapiens
acagcggtaagc

SEQ ID NO: 28 - DNA, 12 bp, Homo sapiens
ccgcagcccatg

SEQ ID NO: 29 - DNA, 24 bp, Artificial
  Synthetic oligonucleotide primer
cgccgctcgccgcagacttacttc

SEQ ID NO: 30 - DNA, 24 bp, Artificial
  Synthetic oligonucleotide primer
aaagctacagggccaggtctgaag

SEQ ID NO: 31 - DNA, 31 bp, Artificial
  Synthetic oligonucleotide primer
tcggcgccatatgcaaagttgtgaatccagt

SEQ ID NO: 32 - DNA, 31 bp, Artificial
  Snythetic oligonucleotide primer
attactcgagagttagtggctcttctcagca

SEQ ID NO: 33 - DNA, 25 bp, Artificial
  Synthetic oligonucleotide primer
gatcccggcggaccatgtgattgtg

SEQ ID NO: 34 - DNA, 24 bp, Artificial
  Synthetic oligonucleotide primer
ctcaggcgggtagaggacatcaaa

SEQ ID NO: 35 - DNA, 21 bp, Artificial
  Synthetic oligonucleotide primer
gccccggggtgtgctaaagag

SEQ ID NO: 36 - DNA, 21 bp, Artificial
  Synthetic oligonucleotide primer
cctgcatgggcgctgtctttg

SEQ ID NO: 37 - DNA, 24 bp, Artificial
  Synthetic oligonucleotide primer
cgcagacttacttccccggctcag

SEQ ID NO: 38 - DNA, 24 bp, Artificial
  Synthetic oligonucleotide primer
ctgcatgggctggttgtataaatc

SEQ ID NO: 39 - DNA, 24 bp, Artificial
  Synthetic oligonucleotide primer
ggatgctaacaggggcgccgtaaa

SEQ ID NO: 40 - DNA, 25 bp, Artificial
  Synthetic oligonucleotide primer
gcagagcaccgtgggtggtggaata

SEQ ID NO: 41 - protein, 13 aa, Artificial
  Hydrophilic synthetic peptide based on sequences of the PAOh1/SMO protein
EEPRGGRWDEDEQ

SEQ ID NO: 42 - protein, 13 aa, Artificial
  Hydrophilic synthetic peptide based on sequences of the PAOh1/SMO protein
EEVRNRIRNDPDD

(19) United States
(12) Patent Application Publication
(10) Pub. No.: US 2004/0229241 A1
(43) Pub. Date: Nov. 18, 2004

(54) Cloned mammalian polyamine oxidase

(76) Inventors: Robert A. Casero, Glenn Aron, MD (US); Yanlin Wang, Baltimore, MD (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/733,020

(22) Filed: Dec. 12, 2003

Related U.S. Application Data
(63) Continuation-in-part of application No. PCT/US02/18666, filed on Jun. 13, 2002.
(60) Provisional application No. 60/297,815, filed on Jun. 13, 2001.

(51) Int. Cl. C12Q 1/68; C07H 21/04; C12N 9/06

(52) U.S. Cl. 435/6; 435/69.1; 435/191; 435/320.1; 435/325; 536/23.2

(57) ABSTRACT

Polynucleotides and the corresponding polypeptides of cloned mammalian polyamine oxidase (PAO) (including various isoforms and truncated forms) are provided. Also provided are antibodies to cloned mammalian PAO, and vectors and host cells containing cloned PAO, and methods for their use.

### BACKGROUND OF THE INVENTION

### FIELD OF THE INVENTION

The invention generally relates to the mammalian polyamine oxidase (PAO) enzyme. In particular, the invention provides cloned mammalian PAO and methods for its use as a diagnostic and prognostic tool.

### BACKGROUND OF THE INVENTION

The polyamines putrescine, spermidine, and spermine are naturally occurring polycationic alkylamines that have been demonstrated to be important in normal and neoplastic cell proliferation, differentiation, and in some cases, cell survival (1-3). Because of the absolute requirement of these compounds for cell growth, the polyamine metabolic pathway (FIG. 1) is a promising target for antiproliferative strategies such as those employed in cancer therapies (4). In fact, several tumor types, including prostate tumors, have been demonstrated to possess disregulated polyamine metabolism. Although much work (1) on interfering with the polyamine metabolic pathway has focused on blocking synthesis by directly inhibiting the biosynthetic enzymes ornithine decarboxylase and S-adenosylmethionine decarboxylase, more recent work has centered on using the self-regulatory properties of polyamine metabolism to design active polyamine analogues. Along this line, several symmetrically and unsymmetrically substituted antitumor polyamine analogues have been synthesized (5, 6).

Interestingly, in addition to regulating the biosynthetic enzymes, some of the most effective antitumor polyamine analogues were found to profoundly increase the catabolism of polyamines (7). In some instances, it appears that the activity of polyamine catabolism is directly associated with activity of the analogues (2, 8, 9).

Polyamine catabolism is mediated by the activity of two enzymes acting sequentially or through the activity of a single oxidase. One rate-limiting enzyme in polyamine catabolism is spermidine/spermine N¹-acetyltransferase (SSAT) (10). This enzyme catalyzes the addition of an acetyl group to the N¹-position of either spermidine or spermine. The acetylated polyamine then becomes the preferred substrate for the activity of acetylpolyamine oxidase (APAO), a flavin adenine dinucleotide-dependent oxidase that results in production of 3-acetamido propanal, H₂O₂, and either spermidine or putrescine, depending on the starting polyamine (11, 12). It should be noted that unmodified spermine is also a substrate of APAO (11). The enzyme polyamine oxidase described in this invention is a FAD-dependent oxidase that can act directly on the unacetylated polyamines and acetylated polyamines in a manner similar to that reported for a plant amine oxidase (14).

As stated above, the polyamine catabolic pathway has been implicated in the sensitivity of several tumor types to many antitumor polyamine analogues. This has been particularly true for SSAT. Several studies (2, 7, 13) have demonstrated that the activity of SSAT can increase several thousand-fold in response to exposure to polyamine analogues. However, it has been thought previously (12) that the activity of polyamine oxidation was constitutive and primarily limited by the availability of the acetylated substrate. Although there are recent cloning reports of yeast and plant PAOs (14, 15), the direct study of the regulation of a mammalian PAO has been hampered by the fact that no clone of the mammalian enzyme has been available.

It would be of great benefit to have available cloned mammalian PAO. Such a cloned form of the enzyme would be desirable for use as a diagnostic and/or prognostic tool, for example, to determine the etiology of and predict optimal treatment regimens for disease states caused by abnormal expression of this enzyme.

### SUMMARY OF THE INVENTION

It is an object of this invention to provide a substantially purified polynucleotide of mammalian origin that encodes the mammalian polyamine oxidase (PAO) enzyme.

According to the invention, the mammalian (in particular human) polynucleotide that encodes the PAO enzyme has been identified, isolated and cloned. The polynucleotide encoding a polypeptide with PAO activity is the product of splicing several exons together. Both isoforms of PAO and truncated forms of PAO have been made, and hosts containing the substantially purified polynucleotides and antibodies to the PAO produced from the substantially purified PAO have been prepared. The resulting proteins from the various clones can oxidize both the N1-acetylated polyamines and the unacetylated polyamines

### BRIEF DESCRIPTION OF THE DRAWINGS

FIG. 1. The polyamine metabolic pathway. AdoMetDC, s-adenosylmethionine decarboxylase; ODC, ornithine decarboxylase; SSAT, spermidine/spermine N¹-acetyltransferase; PAO, polyamine oxidase; APAO, N¹-acetyl polyamine oxidase.

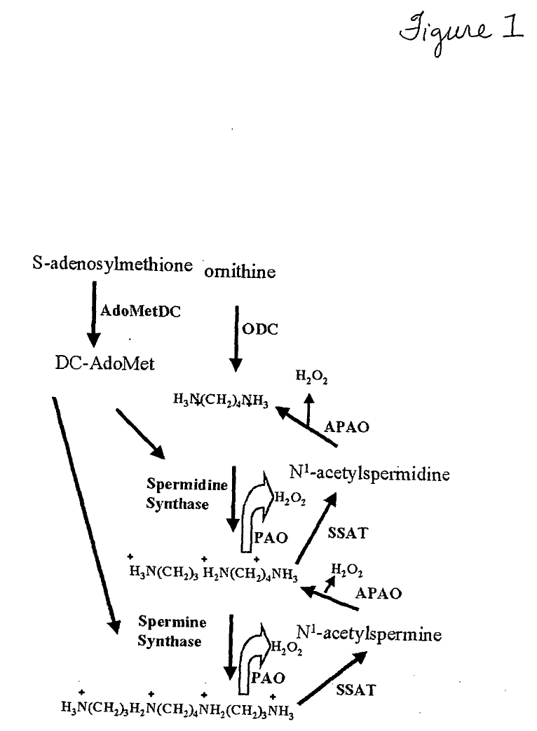

FIG. 2A-C. A, nucleotide (1894 bp, SEQ ID NO. 1) and predicted amino acid (555AA, SEQ ID NO. 2) sequences of the PAOh1. The ATG initiation codon and the TGA stop codon are in boldface. B, genomic structure of the human PAOh1 gene. The seven exons are numbered and represented by filled boxes. C, sequences at exon-intron junctions. Exon sequences are in uppercase letters and intron sequences are in lowercase letters. Sequences are: Exon 1, 5′ splice donor: SEQ ID NO. 17; Exon 1, 3′ splice donor: SEQ ID NO. 18; Exon 2, 5′ splice donor: SEQ ID NO. 19; Exon 2, 3′ splice donor: SEQ ID NO. 20; Exon 3, 5′ splice donor: SEQ ID NO. 21; Exon 3, 3′ splice donor: SEQ ID NO. 22; Exon 4, 5′ splice donor: SEQ ID NO. 23; Exon 4, 3′ splice donor: SEQ ID NO. 24 Exon 5, 5′ splice donor: SEQ ID NO. 25; Exon 5, 3′ splice donor: SEQ ID NO. 26; Exon 6, 5′ splice donor: SEQ ID NO. 27; Exon 6, 3′ splice donor: SEQ ID NO. 28.

FIG. 3A-D. Determination of PAO activity and Km from TnT-produced protein. In vitro transcription and translation were performed with T7-coupled wheat germ extract system. TnT reaction (10 μl) was used for each assay with spermine as the substrate. A, PAO activity from TnT products using pPAOh1 or vector pcDNA3.1 as the template. B, effects of amine oxidase inhibitors on PAO activity in protein from TnT reaction (pPAOh1). The inhibitors used in the experiment: pargyline (monoamine oxidase inhibitor)+semicarbazide (diamine oxidase inhibitor), MDL 72,527 (PAO inhibitor), and a no inhibitor control as indicated. The ordinate is measured in arbitrary fluorescent units/equal amounts of TnT reaction mix. Bars, the mean of at least two trials with a variation of <10%. C, in vitro transcription and translation of human PAOh1 with wheat germ extract system. The labeling assay was performed in the presence of [³⁵S]methionine with 2 μg of linearized plasmid as the template in a 25-μl TnT reaction. The labeled transcription products were then separated by 10% SDS-PAGE. The templates used in the assays were: pPAOh1 or pcDNA3.1 vector as indicated. The arrow indicates the position of PAOh1 protein. D, increasing concentrations of spermine were used with equal amounts of TnT reaction products to determine initial velocity, and Km was determined by a Lineweaver-Burke transformation.

FIG. 4A and B. BENSpm-induced PAOh1 expression in the H157 cell line. A, PAO activity of NCI H157 cells after exposure to 10 mM BENSpm. The ordinate represents pmol H₂O₂ produced/mg protein. Bars, the mean of two experiments with a variation of <10%. B, total RNA (20 μg) from controls or cells that had been treated for 24 h with 10 μM BENSpm was used in each lane for Northern blot analysis with labeled pPAOh1 cDNA as a probe. The blot was boiled and reprobed with 18S ribosomal DNA as a loading control.

FIG. 5. Schematic depiction of PAO-isoform 2 in comparison to PAO-isoform 1. For PAO-isoform 2, the cDNA is 1735 bp long; the open reading frame encodes 502 amino acids; the isoform results from the splicing of 8 exons and 7 introns. The new introns (159 bp) is a portion of exon V of isoform -1.

FIG. 6A and B. Schematic depiction of B, PAO-isoform 3, in comparison to A, PAO-isoform 1. For PAO-isoform 3, the cDNA is 799 bp long; the open reading frame encodes 109 amino acids.

FIG. 7A and B. Schematic depiction of B, PAO-isoform 4, in comparison to A, PAO-isoform 1. For PAO-isoform 4, the cDNA is 1825 bp long; the open reading frame encodes 532 amino acids.

FIG. 8A and B. Schematic depiction of B, PAO-truncation 1 (T-1) in comparison to A, PAO-isoform 1. For T-1, the cDNA is 1073 bp long; the open reading frame encodes 312 amino acids; the truncated portion is bps 971-1791 (amino acids 301-555), 12 new amino acids are added to the C-terminal, and a new stop codon TAG is introduced.

FIG. 9A and B. Schematic depiction of B, PAO-truncation 2 (T-2) in comparison to A, PAO-isoform 1. For T-2, the cDNA is 1171 bp long; the open reading frame encodes 314 amino acids; the truncated portion is bps 959-1681 (amino acids 298-538).

FIG. 10A and B. Schematic depiction of B, PAO-truncation 3 (T-3) in comparison to A, PAO-isoform 1. For T-3, the cDNA is 943 bp long; the open reading frame encodes 238 amino acids; the truncated portion is bps 170-1120 (amino acids 38-354).

FIG. 11A and B. Schematic depiction of B, PAO-truncation 4 (T-4) in comparison to A, PAO-isoform 1. For T-4, the cDNA is 293 bp long; the open reading frame encodes 75 amino acids; the truncated portion is bps 106-1548 (amino acids 13-493).

FIG. 12. Induction of PAO mRNA expression in various lung cancer cell lines by the polyamine analogue BENSpm 10 g of total RNA was hybridized to radiolabeled pPAOh1 probe.

FIG. 13. PAO activity corresponding to cell lines in FIG. 12. Fold increase over untreated cells is as indicated. Where indicated, lung cancer cells were treated with 10 μM BENSpm for 24 hrs.

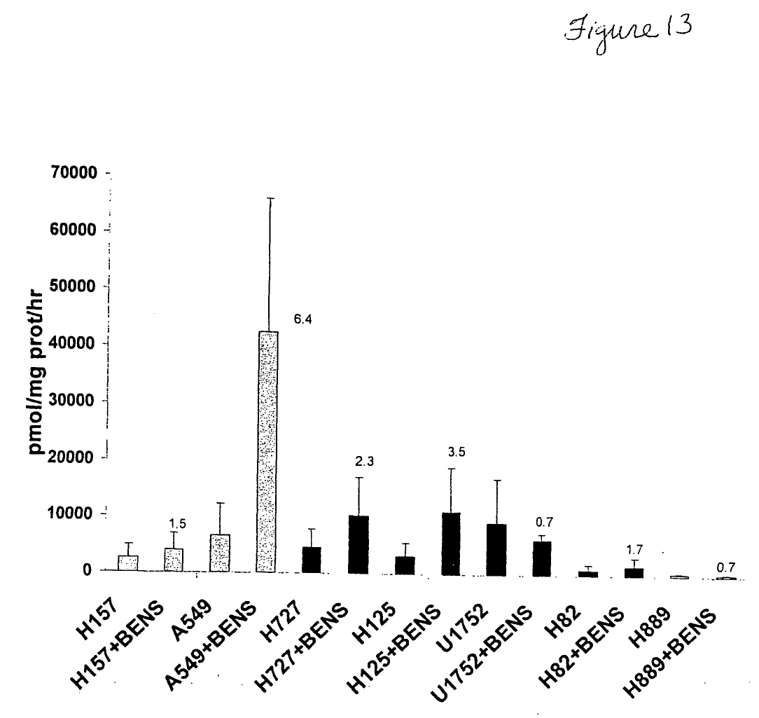

FIG. 14A and B. A, Nucleic acid sequence of PAO Isoform 1, SEQ ID NO. 1; B, amino acid sequence of PAO Isoform 1, SEQ ID NO. 2.

FIG. 15A and B. A, Nucleic acid sequence of PAO Isoform 2, SEQ ID NO. 3; B, amino acid sequence of PAO Isoform 2, SEQ ID NO. 4.

FIG. 16A and B. A, Nucleic acid sequence of PAO Isoform 3, SEQ ID NO. 5; B, amino acid sequence of PAO Isoform 3, SEQ ID NO. 6.

FIG. 17A and B. A, Nucleic acid sequence of PAO Isoform 4, SEQ ID NO. 7; B, amino acid sequence of PAO Isoform 4, SEQ ID NO. 8.

FIG. 18A and B. A, Nucleic acid sequence of PAO Truncation T-1, SEQ ID NO. 9; B, amino acid sequence of PAO Truncation T-1, SEQ ID NO. 10.

FIG. 19A and B. A, Nucleic acid sequence of PAO Truncation T-2, SEQ ID NO. 11; B, amino acid sequence of PAO Truncation T-2, SEQ ID NO. 12.

FIG. 20A and B. A, Nucleic acid sequence of PAO Truncation T-3, SEQ ID NO. 13; B, amino acid sequence of PAO Truncation T-3, SEQ ID NO. 14.

FIG. 21A and B. A, Nucleic acid sequence of PAO Truncation T-4, SEQ ID NO. 15; B, amino acid sequence of PAO Truncation T-4, SEQ ID NO. 16.

FIG. 22. Exon structures of human PAO isoforms The internal exon present in exon V of PAOh1 can act as an intron and is spliced out of PAOh2 and PAOh4, resulting in exons Va and Vb. PAOh4 contains a newly identified exon VIa.

FIG. 23A and B. Kinetic properties of human PAO isoforms (A) In this Table all values represent means for at least two experiments, each with all samples prepared and measured in duplicate from the same TNT reaction. Vₘₐₓ(‘V MAX’) and Km′ (‘K M’) values were predicted using the Lineweaver-Burk transformation of the Michaelis-Menten equation. Vₘₐₓ units are presented as pmol of H₂O₂ generated/min per unit of protein, with protein unit determination via SDS/PAGE analysis of a radiolabeled aliquot of the TNT reaction mixture. (B) Shows representative double-reciprocal plots of PAOh1 versus PAOh4 using increasing values of Spm as substrate.

FIG. 24. Specific activities of PAO isoforms with various polyamine substrates Protein produced from parallel TNT reactions in the presence and absence of [³⁵S]methionine was used for PAO activity analysis (shown) or quantified by SDS/PAGE (not shown). Band intensities from SDS-PAGE were normalized for the methionine content of each isoform and used for determination of specific activity values.

FIG. 25. Antitumor polyamine analogues and PAOh1 inhibitor used in this study.

FIG. 26. Induction of PAOh1/SMO activity by BENSpm in human lung cancer cells. Seven human lung cancer cell lines representative of the major forms of lung cancer were exposed to 10 1MBENSpm for 24 h to determine the e.ect on PAOh1/SMO activity. The numbers above each cell line represent the fold-increase in activity induced by BENSpm over untreated basal activity. The cell lines are: 1 H157 untreated, 2 H157+BENSpm, 3 A549 untreated, 4 A549+BENSpm, 5 H727 untreated, 6 H727+BENSpm, 7 H125 untreated, 8 H125+BENSpm, 9 U1752 untreated, 10 U1752+BENSpm, 11 H889 untreated, 12 H889+BENSpm, 13 H82 untreated, 14 H82+BENSpm. Values are the means±SE of two trials performed in duplicate using 250 μM spermine as the substrate.

FIG. 27A,B. Time- and dose-dependent changes in PAOh1/SMO activity in NCI A549 non-small-cell lung cancer cells in response to treatment with BENSpm or CPENSpm. A Cells were exposed to 10 μM BENSpm or CPENSpm for up to 24 h. B Cells were exposed to increasing concentrations of either BENSpm or CPENSpm for 24 h. The results in both A and B are the means±SE of four separate experiments using 250 μM spermine as substrate performed in duplicate

FIG. 28A, B. Time- and dose-dependent changes in steady-state PAOh1/SMO mRNA levels in NCI A549 non-small-cell lung cancer cells in response to treatment with BENSpm or CPENSpm. A Increase in PAOh1/SMO mRNA with increasing exposure time to 10 μM analogue. B Increase in PAOh1/SMO mRNA with increasing concentrations of analogue for 24 h. Fold increase is relative to untreated controls. Each point represents the mean±SE of two experiments performed in duplicate

FIG. 29A, B. Effects of cotreatment of NCI A549 cells with PAOh1/SMO-inducing analogues and the PAOh1/SMO inhibitor MDL 72,527. Cells were seeded at 5×10³ cells/well and treated for 96 h with (A) 10 μM BENSpm or (B) 10 μM CPENSpm in the presence or absence of 250 μM MDL 72,527 (MDL). Each point represents the mean±SD of triplicate determinations. Note that the error bars fall within the symbol at each point

FIG. 30. Substrate specificity of PAOh1/SMO activity induced by BENSpm and CPENSpm. NCI A549 cells were treated with 10 μM of either BENSpm or CPENSpm for 24 h. Cell lysate from the treated cells were then assayed for PAOh1/SMO activity using 250 μM spermine, spermidine, or N1-acetylspermine. The data represent the means of four separate experiments performed in duplicate±SE.

FIG. 31. Ability of various polyamine analogues to induce PAOh1/SMO activity. NCI A549 cells were exposed to 10 μM of the indicated polyamine analogues for 24 h. PAOh1/SMO activity was then measured in the corresponding cell lysates using 250 μM spermine as the substrate. The basal oxidase activity was 4184 pmol/mg per hour. It should be noted that 250 μM MDL 72,527 (MDL) only reduced the basal activity to 4055 pmol/mg per hour. The PAOh1/SMO inhibitor MDL 72,527 was used at a concentration of 250 μM where indicated (MDL) in the assay of the analogue-induced PAOh1/SMO to determine if this induced activity could be efficiently inhibited. The results presented are the means±SE of four separate experiments performed in duplicate.

FIG. 32. Symmetric, unsymmetric, conformationally restricted, and oligamine analogues used.

FIG. 33A, B. A, Substrate specificity of purified recombinant PAOh1/SMO. Purified protein was incubated in the presence of 250 μM of the indicated substrate (Spm, spermine; N¹-Aspm, N¹-acetylspermine; and Spd, spermidine). The oxidase inhibitor, MDL 72,527 was used at the concentration of 250 μM. The data are a representative experiment performed in triplicate with the error bars indicating the standard deviation. B, Inhibition of PAOh1/SMO activity by polyamine analogues. Purified protein was incubated in the presence of 250 μM spermine and 10 μM of the indicated analogue. Each bar represents the mean of triplicate determinations with indicated standard deviation.

FIG. 34A, B. Dose-response to oligamine inhibitors of PAOh1/SMO. (A) Increasing concentrations of the indicated inhibitors were incubated with purified protein in the presence of 250 μM spermine. CHENSpm was also included since this analogue has been demonstrated to inhibit the plant PAO (32). (B) Increasing concentrations of the oxidase inhibitor MDL 72,527 were incubated with purified protein in the presence of 250 μM spermine. Each point represents the mean of duplicate determinations.

FIG. 35. Exon structures of human PAO isoforms As shown in FIG. 22, but with primer positions for real time PCR indicated.

### DETAILED DESCRIPTION OF THE PREFERRED EMBODIMENTS OF THE INVENTION

The present invention provides the first substantially purified polynucleotide of mammalian origin encoding a polypeptide with polyamine oxidase (PAO) activity, and the polypeptide encoded thereby, i.e. a mammalian PAO enzyme. The polynucleotide may be a polydeoxyribonucleotide (DNA) or a polyribonucleotide (RNA). By “substantially purified” we mean that the polynucleotide has been isolated from a mammalian source and cloned using genetic engineering techniques. In one embodiment of the invention, the polynucleotide is from a human source. In a preferred embodiment, the sequence of the polynucleotide is SEQ ID NO.1, SEQ ID NO.3, SEQ ID NO.5, SEQ ID NO.7, SEQ ID NO.9, SEQ ID NO.11, SEQ ID NO. 13, and SEQ ID NO. 15, and the primary amino acid sequence of the corresponding polypeptides (i.e. the translation product of the polynucleotide, a human PAO enzyme) are SEQ ID NO. 2, SEQ ID NO.4, SEQ ID NO.6, SEQ ID NO.8, SEQ ID NO.10, SEQ ID NO.12, SEQ ID NO. 14, and SEQ ID NO.16, respectively.

It should be noted that the polyamine oxidase protein and activity referred to in this application arise specifically from the PAOh1/SMO gene. The products of this gene are truly polyamine oxidases in that they preferably oxidize the unacetylated polyamine spermine with the greatest affinity for spermine. The PAOh1/SMO polyamine oxidase gene products should not be confused with the previously characterized N¹-acetylpolyamine oxidase that preferentially oxidizes N¹-acetylspermine and N-acetlyspermidine, products of the polyamine catabolic enzyme spermidine/spermine N¹-acetyltransferase. This acetylpolyamine oxidase is sometimes also referred to as PAO.

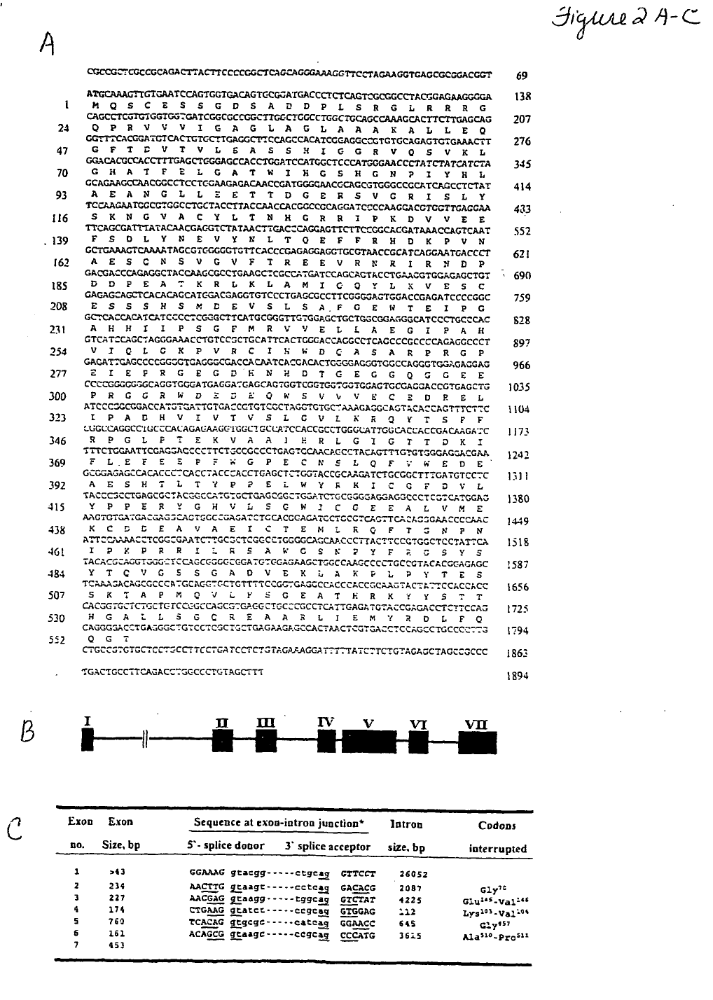

As disclosed herein, the polynucleotide encoding a polypeptide with PAO activity is the product of the splicing of several exons. For example, SEQ ID NO. 1 is the product of the splicing of seven exons (see FIG. 2). As is well known to those of ordinary skill in the art, such splicing reactions often exhibit variability, i.e. different combinations of the available exons are joined together, resulting in polypeptides which differ in primary sequence. These polypeptides with differing but related primary sequences are known as “splice variants” of mammalian PAO. Typically, such splice variants have several regions of primary amino acid sequence that are identical, whereas others regions may be omitted or exchanged. For example, if gene contains exons A, B, and C, splice variants of the gene could, theoretically, be ABC, AB, AC, or BC. However, as is recognized by those of ordinary skill, some splice variants may be more likely to occur than others for any of several reasons, e.g. developmental regulation of the splicing reaction, conformation of the DNA, evolutionary selective pressure against splice variants that are inactive or overly active, etc. All such splice variants of mammalian PAO are intended to be included in the scope of the present invention, whether they are naturally occurring or constructed in a laboratory setting using genetic engineering techniques. Such splice variants are referred to herein as “isoforms of PAO” or simply as “isoforms”.

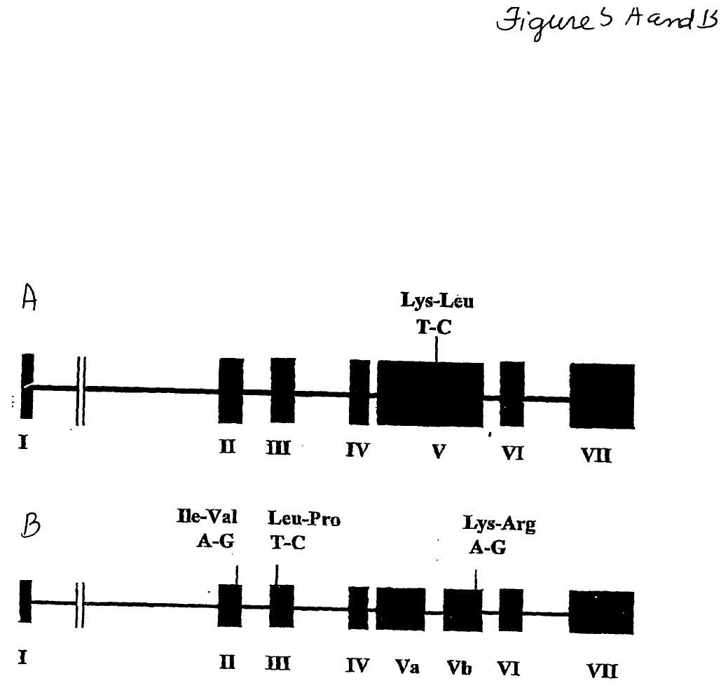

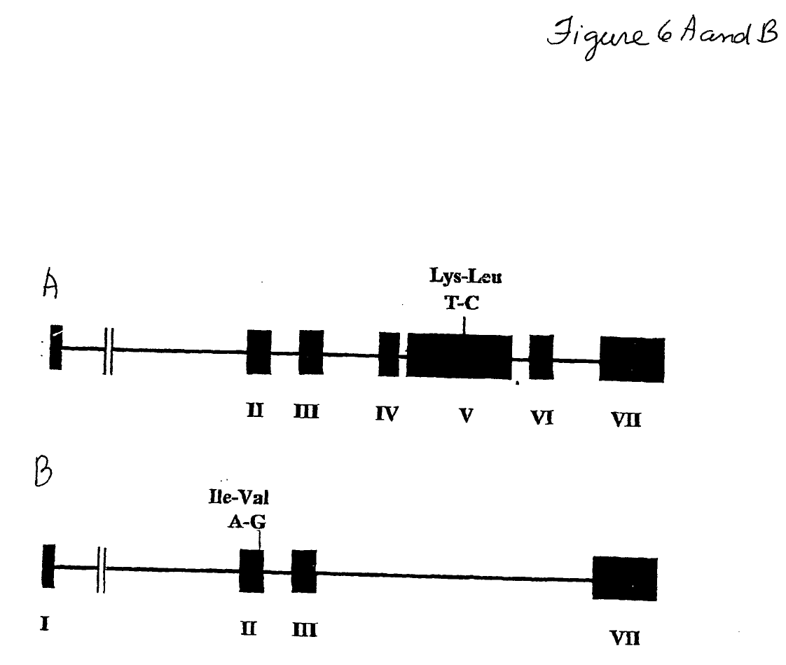

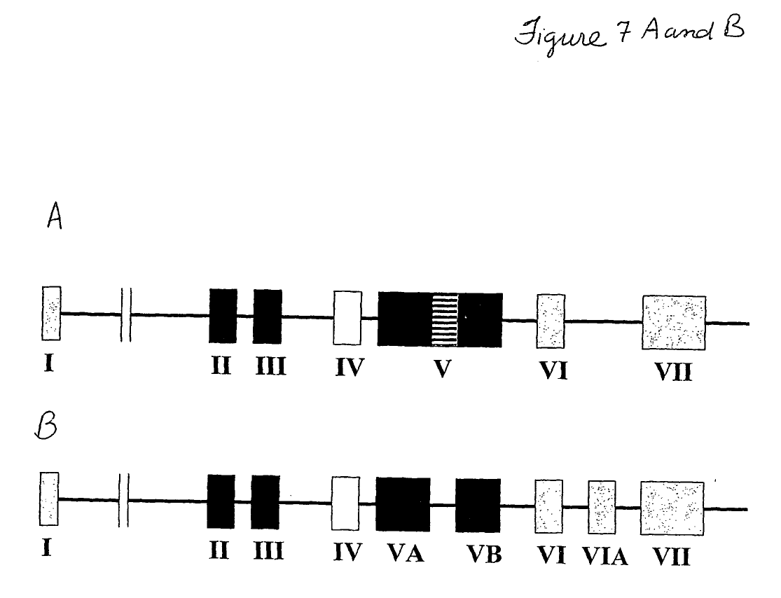

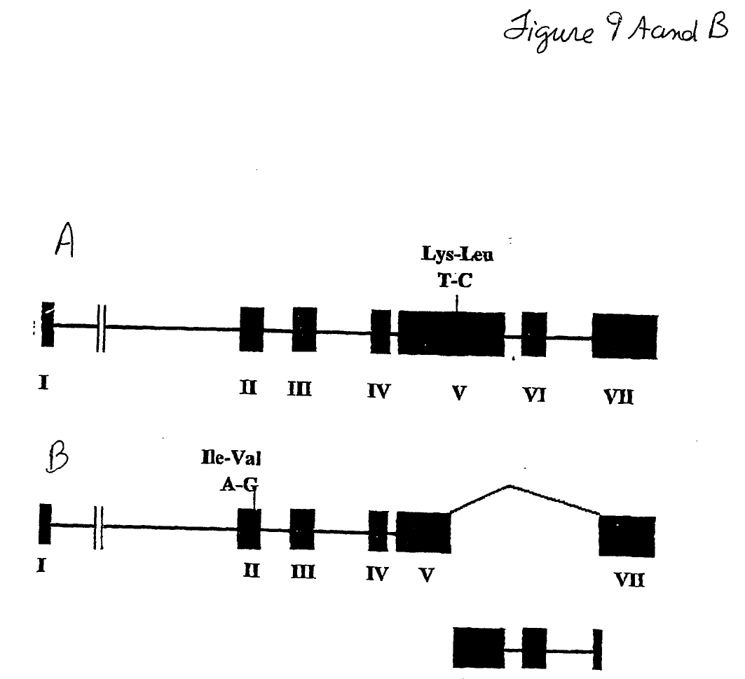

Further, as described herein, mammalian PAO is also present in cells in several truncated forms which display PAO activity (see Example 4, and FIGS. 5-9). Such truncated forms are also intended to be within the scope of the present invention, and are referred to as “truncations” or “truncated forms of PAO” or simply as “truncations” or “truncated forms”.

In a preferred embodiment of the present invention, the polynucleotide which encodes the mammalian PAO is SEQ ID NO.1, SEQ ID NO.3, SEQ ID NO.5, SEQ ID NO.7, SEQ ID NO.9, SEQ ID NO.11, SEQ ID NO. 13, or SEQ ID NO. 15 or modified variants thereof, and the encoded polypeptide is SEQ ID NO. 2, SEQ ID NO.4, SEQ ID NO.6, SEQ ID NO.8, SEQ ID NO.10 or SEQ ID NO.12, SEQ ID NO. 14, or SEQ NO. ID 16, or modified variants thereof. The term “modified variants” refers to both nucleic acid sequences and to corresponding translated amino acid sequences. As will be readily appreciated by those of skill in the art, it is possible to make changes of several types in a DNA sequence and still obtain the same (or a functionally equivalent) protein translation product. For example, due to degeneracy in the DNA code, it is possible to alter the DNA sequence and still encode an identical polypeptide (a “silent variation”).

Alternatively, it is possible to make changes in the DNA sequence which result in conservative substitutions of functionally similar amino acids in the encoded polypeptide. Still further, changes resulting in non-conservative amino acid substitutions may also be made which, depending on the nature and location of the substitution, may have no deleterious effect on the activity of the polypeptide, or may have another desired effect (such as increasing or decreasing thermal stability, susceptibility to protease degradation, solubility, etc.) with or without impacting the primary enzymatic activity of the polypeptide. In some cases, it may be acceptable to cause an alteration in the enzymatic activity of the polypeptide in order to achieve another desirable outcome. Further, more extensive modifications are also be contemplated in the scope of the invention. For example, the DNA sequence may be shortened to remove sequences, or sequences may be added to the DNA for any of several reasons. For example, it may be desirable to modify the DNA to effect changes in the DNA itself (e.g. to introduce convenient restriction sites for manipulation of the DNA; to add, remove or rearrange cis- and/or trans-acting elements such as promoters, enhancers, etc.; to increase or decrease the Tm of the DNA, to alter its conformation, to alter its hybridization properties, or may be used in antisense strategies, etc. Alternatively, it may be desirable to modify the DNA in order to effect a change in the corresponding polypeptide, e.g. to introduce or remove glycosylation sites, to add signal peptides, to add tagging or reporter moieties (e.g. green fluorescent protein), to create other desirable chimeric proteins, to decrease the number of amino acids or to generate polypeptide fragments for purposes such as, for example, raising antibodies, etc. Similarly, post-translational variants of the polypeptide are also encompassed by the scope of the present invention; for example, post-translational modification such as protease digestion, or various chemical modifications such as acetylation or amidation, are contemplated. All such modified variants of the sequences disclosed herein are intended to be encompassed by the present invention. In some embodiments, the resulting modified variant produces or is a polypeptide having polyamine oxidase activity of at least about 25% to 100% (or greater) of that of the sequences disclosed herein, and preferably having polyamine oxidase activity of at least about 50% to 100% (or greater) of that of the sequences disclosed herein. An exception is the generation of fragments for use as, for example but not limited to, probes (e.g. DNA fragments) or to raise antibodies (e.g. polypeptide fragments), which may be useful in the practice of the present invention and may exhibit little or no PAO activity by themselves. In general, modified variants will exhibit nucleic acid homology of from about 50% to about 100% compared to that of the starting material (i.e. to that of the sequences disclosed herein or fragments thereof, and preferably from about 75% to about 100% to that of the sequences disclosed herein or fragments thereof). Those of skill in the art will recognize that this percentage would exclude sequences not originally derived from PAO sequences, for example, sequences added via genetic engineering during construction of DNA encoding a chimeric protein, or which contain vector or regulatory sequences, etc. Similarly, modified variants of the polypeptide will preferably exhibit amino acid homology in the range of from about 50 to 100% compared to the starting material, (the sequences disclosed herein or fragments thereof) and most preferably from about 75 to 100%, excluding non-related sequences that are added, for example, during construction of a chimeric protein. If the modified variant is a fragment of the original DNA or protein sequence, such a DNA fragment will typically be at least about 20 nucleotides in length, and a polypeptide fragment will be about at least about 10 amino acids in length. Further, such modified variants may be the result of deliberate changes introduced in a laboratory setting, or fortuitous mutations which occur in a laboratory setting, or may be natural mutations or variants, for example, variations in sequence between individuals or species.

The polynucleotide and polypeptide sequences which are the subject of the present invention may be either derived from natural sources (i.e. isolated and purified directly from a mammalian source); or they may be produced by genetic engineering techniques (e.g. by PCR, in vitro translation systems, bacterial expression systems and the like), or by chemical synthetic methods, all of which techniques are well-known to those of skill in the related arts.

In one embodiment of the invention, the mammalian PAO enzyme is of human origin. However, those of skill in the art will recognize that all mammalian species possess PAO enzymes, and all such substantially purified enzymes and the nucleic acid sequences which encode them are intended to be encompassed by the scope of the present invention.

Uses of the polypeptides of the present invention include but are not limited to, for example, providing such polypeptides (or suitable fragments thereof ) to a cell in order to modulate the expression of PAO in the cell. For example, in a cell in which PAO is not expressed, or expressed at a very low level, or in which a nonfunctional form of PAO is expressed, the provision of a functional form of the enzyme may alleviate disease conditions resulting from the lack of a normal form of the enzyme.

The present invention also provides vectors comprising a substantially purified polynucleotide of mammalian origin encoding a polypeptide with PAO activity, or a fragment thereof. Those of skill in the art are well acquainted with techniques of genetic engineering by which polynucleotides encoding entire proteins, or encoding selected regions of the proteins, can be identified and placed within suitable vectors. Such vectors are useful for various reasons, for example, in order to carry out in vitro translation of the encoded polypeptide, or for use in maintaining the polynucleotide in a convenient form for various manipulations, such as for the transformation of host cells.

The polynucleotide sequences of the present invention may be used as probes, for example, to detect PAO DNA and/or mRNA within cells of interest. Those of skill in the art will recognize that for use as a probe, it is frequently not necessary to utilize an entire coding region of a gene. Rather, short regions of a gene sequence may suffice, particularly those regions known to possess high homology between many individuals of a sample population. In fact, non-coding intron regions may also be employed as probes, so long as they are sufficiently unique to identity the target gene. The rationale for selecting such regions and the methods of designing, making and using such probes are well known to those of skill in the art, and include taking into account such factors as probe length, secondary structure of the ssDNA, Tm of the ds hybridized sequences, etc. Guidance concerning such considerations in probe design is well known and readily available to those of skill in the art and dependent on the specific use, for example, standard Southern and Northern blotting, in situ hybridization, RNase protection, etc. n general, a probe based on the PAO gene of the present invention will be in the range of about 26 nts in length to about 1000 nts in length, depending on the technique used. Further, preferred regions of the gene to target include but are not limited to: the FAD binding region, exons 1-3, and probes that specifically recognize the individual splice variants.

The present invention also provides methods directed to the use of such probes for detecting PAO-related DNA or RNA in a cell of interest. By PAO-related DNA or RNA, we mean that the probe may be utilized to detect (typically by hybridization with complementary nucleic acid sequences) either DNA which encodes the PAO gene, or RNA (e.g. mRNA, either spliced or unspliced) which encodes the PAO protein. Typically, DNA or RNA is isolated from cell of interest (e.g. tumor cells, or cells which may be predisposed to become neoplastic) and the isolated DNA or RNA is incubated with the probe molecules under conditions inducing denaturation, followed by hybridization of complementary sequences. Because the probe molecules would typically be labeled, (for example, with radioactivity) it would be possible to detect DNA or RNA from the cell which became hybridized to a probe. Alternatively, pairs of or single DNA fragments homologous to regions of a gene of interest may be constructed and utilized as primers in, for example, a PCR reaction to amplify regions of DNA flanked by the primers. Such methodologies are routinely utilized by those of skill in the art, and many protocols are readily available for their execution.

Such methods as those of the present invention are useful for many purposes, including but not limited to the detection of PAO DNA within cells (e.g. to detect mutations within the PAO gene of a cell which may predispose the cell to the development of a disease phenotype, for example, cancer); to detect levels of expression of mRNA which encodes a PAO enzyme or fragment(s) thereof (e.g. in order to detect abnormal levels of expression of the enzyme, or the expression of abnormal forms of the enzyme, which might predispose a cell to develop an abnormal disease condition such as cancer); or to monitor the level of expression of PAO mRNA in cells in response to a treatment regimen intended to modulate PAO expression, or to determine the predominant splice variants expressed.

In order to carry out the practice of the present invention, the polynucleotides of the present invention may be placed in vectors which are maintained in host cells. Those of skill in the art are well acquainted with the transformation of host cells with DNA encoding a polypeptide of interest. Types of host cells that are utilized routinely by those of skill in the art include but are not limited to bacteria, yeast, various mammalian cells (e.g. established mammalian cell lines), and insect cells (e.g. Drosophila spp.). Such host cells may also possess utility as therapeutic agents, for example, in order to provide a desired form of a mammalian PAO gene to a cell of interest as in gene therapy. For example, it would be possible to provide a normal form of the enzyme to cells which either do not produce PAO, or which produce an abnormal form of PAO. Likewise, it would be possible to repress expression of a form of PAO by providing to a cell of interest antisense DNA or RNA to block the translation of mRNA encoding a form of PAO.

The present invention also provides antibodies to polypeptides encoding a mammalian PAO enzyme, or fragment thereof. The production of antibodies (both mono- and polyclonal) are well known to those of skill in the art. Such antibodies may be generated to any isoform or truncation of a mammalian PAO enzyme, or to fragments thereof. In a preferred embodiment, such antibodies are generated against SEQ ID NO. 2, SEQ ID NO.4, SEQ ID NO.6, SEQ ID NO.8, SEQ ID NO.10, SEQ ID NO.12, SEQ ID NO.14, and SEQ ID NO. 16, or fragments thereof. The uses of such antibodies include but are not limited to the detection of PAO within cells (for example, the detection of different splice variants of PAO, or of mutant or otherwise abnormal forms of the enzyme). Alternatively, the antibodies may be useful for inhibiting the enzymatic activity of mammalian PAO and various forms thereof upon administration of the antibodies to cells of interest, e.g. diseased cells known to exhibit abnormal polyamine oxidase-related metabolism, or cells which may have a predisposition to development of a diseased phenotype due to such abnormal metabolism.

As described above, various aspects of the present invention may be useful in order to identify abnormalities in the forms or expression of various forms of mammalian PAO. As such, the invention provides diagnostic, prognostic and therapeutic tools for disease conditions associated with such abnormalities. For example, the probes and antibodies of the present invention may be used to identify abnormal forms or abnormal expression levels of PAO in a cell of interest, e.g. in a cancer cell, in order to confirm a diagnosis of malignancy or predict the likelihood of the development of malignancy beforehand. In particular, such methods may help to characterize a disease state, e.g. the potential aggressiveness of a tumor, early in (or even prior to) diagnosis based on the elucidation of the precise type of abnormality. For example, the expression of specific splice variants may be associated with a predisposition to disease conditions. The ability to detect these forms prior to the onset of other symptoms of the disease would clearly be a boon to physicians. Also, the ability to monitor the expression of specific forms of PAO associated with disease conditions during and after therapeutic treatment regimens would be of great utility.

In particular, it is known that the prostate possesses the highest concentration of natural polyamines of any human tissue, and that abnormalities in polyamine metabolism are implicated in prostate cancer. Detection and modulation of PAO in prostate cells may be utilized prophylactically (for early detection of or to prevent the occurrence of prostate cancer) or therapeutically, to treat prostate cancer. For example, up-regulation of POA expression within cells is known to result in increased production of H₂O₂, leading to apoptosis of the cells. Therefore, the PAO enzyme is an excellent target for modulation in order to induce apoptosis in cells of interest.

### EXAMPLES

Materials and Methods

Chemicals. The radionucleotides ([α-³²P]dCTP and [α-³⁵S]methionine) were supplied by Amersham Pharmacia Biotech (Piscataway, N.J.). The TnT coupled wheat germ extract system was purchased from Promega (Madison, Wis.). The TA cloning kit was purchased from Invitrogen (Carlsbad, Calif.). Trizol total RNA reagent was from Life Technologies, Inc. (Rockville, Md.). Advantage cDNA Polymerase Mix system and a retroviral placenta cDNA library were from Clontech Laboratories, Inc. (Palo Alto, Calif.). Restriction and DNA modifying enzymes were purchased from New England Biolabs, Inc. (Beverly, Mass.), Life Technologies, Inc., and Sigma Chemical Co. (St. Louis, Mo.). Life Technologies, Inc. synthesized all of the oligomers used in the experiments.

The DNA sequencing was performed with a Perkin-Elmer ABI Automated DNA Sequencer. N¹,N⁴-bis(2,3-butadienyl)-1,4-butanediamine (MDL 72,527) was kindly supplied by Dr. Eugene Gerner (University of Arizona, Tucson, Ariz.). Other chemicals came from Sigma Chemical Co., Roche Molecular Biochemicals (Indianapolis, Ind.), Bio-Rad (Hercules, Calif.), Aldrich Chemical Company, Inc. (Milwaukee, Wis.), and J. T. Baker, Inc. (Phillipsburg, N.J.).

Cloning of Human PAO. PCR (Advantage cDNA Polymerase Mix system) was used to clone human PAO cDNA. PCR was performed with a gene-specific primer pair [5′-CGCCGCTCGCCGCAGACTTACTTC-3′ (SEQ ID NO. 29) and 5′-AAAGCTACAGGGCCAGGTCTGAAG-3′ (SEQ ID NO. 30)] and cDNA from a human placenta library. The PCR products were then cloned into pCR2.1 vector (pCR2.1/PAOhx).

To construct the pPAOh1 plasmid, the cDNA insert in pCR2.1/PAOh1 was removed by cutting with HindIII and EcoRV and then inserting the resultant fragment into pcDNA3.(−) vector in the same restriction sites.

In Vitro Transcription and Translation. In vitro transcription and translation reactions were performed with the TnT-coupled wheat germ extract system. Parallel reactions were prepared by adding an unlabeled amino acid mixture to one reaction and a [³⁵S]methionine containing amino acid mixture to the other, according to the supplied protocol. Vector pcDNA3.1 and pPAOh1 were linearized by SalI restriction and served as the templates. The labeled translation reactions products were separated by 10% SDS-PAGE, and radioactivity on the labeled PAO band was determined by Phosphor image analysis using Image Quant software (Molecular Dynamics, Sunnyvale, Calif.).

RNA Purification and Northern Blot Assay. Total cellular RNA from the NCI H157 cell line was extracted using Trizol total RNA reagent according to the protocol from the manufacturer. Total RNA (20 μg) was separated on a denaturing 1.5% agarose gel containing 6% formaldehyde, transferred to Zetaprobe membrane (Bio-Rad), and hybridized with a random primer-labeled pPAOh1 cDNA as the probe. Blots were washed and reprobed with an 18S ribosomal cDNA probe as a loading control.

Determination of PAO Enzyme Activity. The cultured H157 cells with or without treatment of 10 μM N¹, N″-bis(ethyl)norspermine (BENSpm) were homogenized with a Dounce tissue homogenizer in ice-cold 0.083 M sodium borate buffer (pH 9.0). The PAO activity in homogenates was assayed by the method of Suzuki et al. (16), which measures the H₂O₂ formed due to oxidation of spermine by converting homovanillic acid into a highly fluorescent compound in the presence of horseradish peroxidase. The samples were prepared in a 600-μl reaction containing 83 mM sodium borate buffer (pH 9.0), 0.04 mg of horseradish peroxidase, 100 μl of cell homogenate, 0.1 mg of homovanillic acid, and 250 μM spermine. Before the addition of homovanillic acid and spermine, the tubes were preincubated for 20 min with shaking at 37° C. to remove endogenous substrates of H₂O₂-producing enzymes. After preincubation, homovanillic acid and spermine were added, and the reactions were incubated for 1 h at 37° C. The enzyme activity was stopped by the addition of 2.0 ml of 0.1 M NaOH solution. The fluorescence intensity was measured with excitation at 323 nm and emission at 426 nm. Background fluorescence was determined by addition of the spermine substrate into the reaction mixture only after inactivation of the enzyme by NaOH. Protein content of the cellular homogenate was determined using the Bio-Rad protein assay kit (Bio-Rad). One unit of PAO activity in cell homogenate was defined as that amount that transformed 1 pmol spermine/mg cell protein/60 min at 37° C. For determination of PAO activity in the product of the unlabeled TnT reaction, 10 μl of TnT reaction were used in the place of the homogenate. The PAO activity in the TnT reactions was represented by the fluorescent compound formed within the 1-h incubation. In some tests, the inhibitor for monoamine oxidase (pargyline), diamine oxidase (semicarbazide), or polyamine oxidase (MDL-72527) was used at the final concentration of 1.0 mM, 0.1 mM, and 0.25 mM, respectively. The concentrations chosen for each inhibitor were based on studies published previously (12).

Determination of Km. The apparent Km of PAO using spermine as a substrate was determined using the TnT-produced protein described above. Concentrations of spermine used were similar to those reported by Libby and Porter (11).

### EXAMPLE 1

### Molecular Cloning of Human PAO

The maize PAO was cloned recently by Tavladoraki et al. (14). Using the information provided by their work, PCR primers spanning the putative flavin adenine dinucleotide-binding site were made to be used in the PCR techniques.

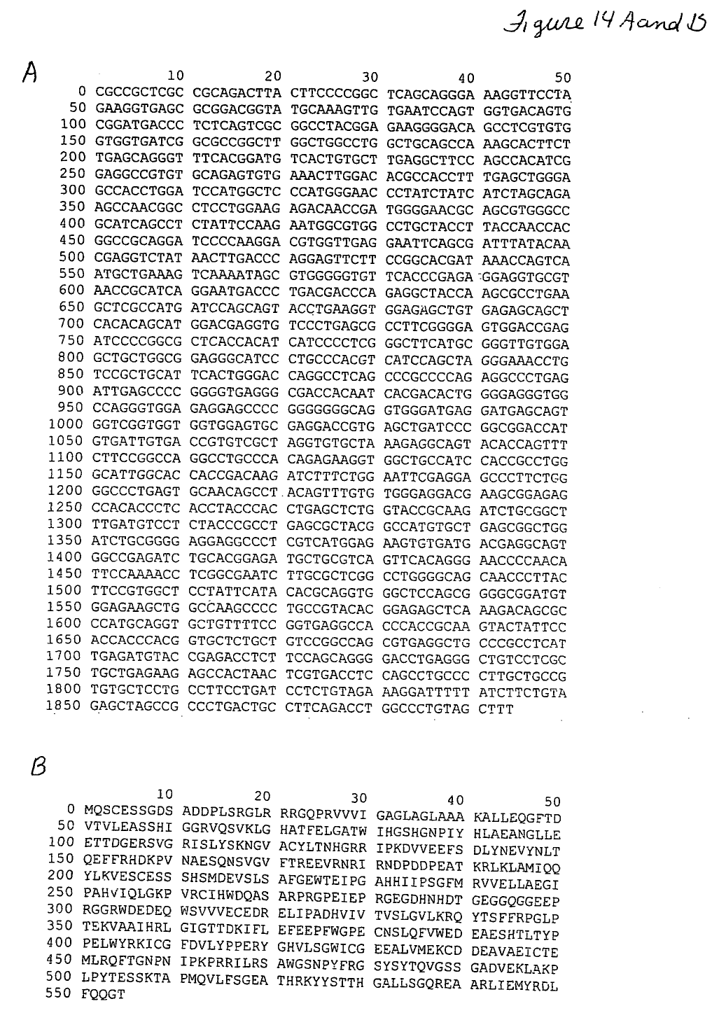

Using human placenta cDNA as a starting material, multiple homologues of the maize PAO were identified. After sequencing of each individual clone, BLAST homology searching of the National Center for Biotechnology Information human genome database revealed that the multiple clones were encoded by the same genomic sequence (accession no. AL1216785), which is located on chromosome 20p13. The longest clone, pPAOh1, is a total of 1894 bp (FIGS. 2A and 14A, SEQ ID NO. 1) and possesses an open reading frame of 1668 bp coding for a putative protein of 555 AA (FIGS. 2A and 14B, SEQ ID NO. 2). This clone was chosen for further characterization. On the basis of the available GenBank data, pPAOh1 is the product of seven exons and six introns (FIG. 2B) spanning 38.9 kb of genomic DNA. The nucleotide sequence representing SEQ ID NO. 1 has been submitted to the GenBank and has the assigned accession no. AY033889.

### EXAMPLE 2

### Activity and Km of in Vitro Transcription/Translation Product

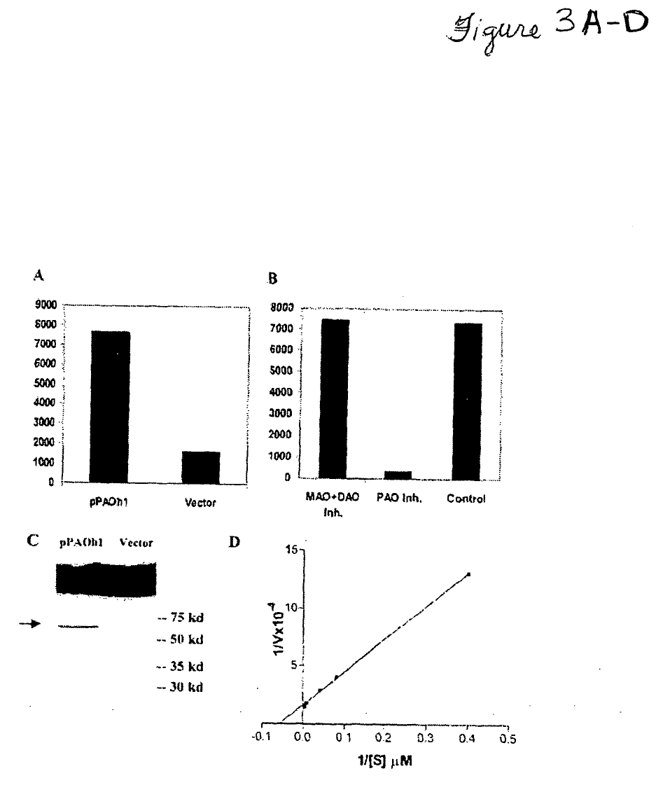

To verify that the newly cloned cDNA coded for a protein that possessed PAO activity, the in vitro TnT wheat germ extract system (Promega) was used. The wheat germ system, rather than the rabbit reticulocyte system, was used because the fluorescent enzyme assay used to detect PAO activity is based on H₂O₂ production by PAO. The rabbit reticulocyte system contains heme, which would result in the Fenton catalysis of H₂O₂. Enzyme activity was determined by the method of Suzuki et al. (16) using spermine as a substrate. Spermine was chosen because the acetylated polyamines are no longer available from a commercial source. Previous work (11, 17) has validated spermine as a PAO substrate. In the standard fluorescence detection assay, the in vitro produced protein demonstrated significant oxidase activity using 250 μM spermine as the substrate (FIG. 3A). To verify that the enzyme activity was attributable to PAO and not monoamine or diamine oxidases, specific inhibitors of each were included in the indicated reactions. Only the PAO inhibitor, MDL 72,527, was effective in inhibiting the human PAOh1 protein product. (FIG. 3B). To ensure equal additions of protein to the assays described above, parallel TnT reactions for each condition were prepared by adding an unlabeled amino acid mixture to one reaction and an [³⁵S]methioninecontaining amino acid mixture to the other. Protein produced in this manner yielded a major band of ˜62 kDa after denaturing PAGE, consistent with the expected size of the open reading frame (FIG. 3C). To determine the apparent Km for the in vitro produced human PAO, increasing concentrations of spermine were used in the calculation of initial velocities of H₂O₂ production as described above. The initial velocity of the reaction was determined for increasing concentrations of spermine ranging from 2.5 to 250 mM. The apparent Km of the TnT-produced PAO using spermine as the substrate was determined by the Lineweaver/Burke transformation to be ˜18 μM (FIG. 3D).

### EXAMPLE 3

### Effects of BENSpm Treatment on PAO mRNA Expression and Enzyme Activity in NCI H157 Cells

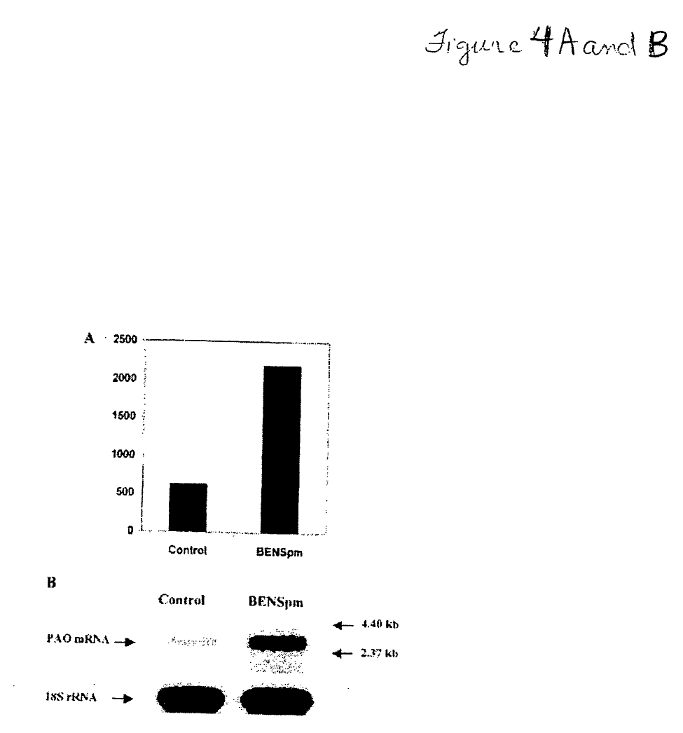

PAO has frequently been described as a constitutively expressed protein. To test this hypothesis, NCI H157 cells were exposed to 10 μM BENSpm for 24 h. This time and concentration were chosen because BENSpm has demonstrated the ability to highly induce SSAT in H157 cells and produce H₂O₂-related apoptosis (3). BENSpm exposure resulted in ˜5-fold increase in PAO message (FIG. 4A) and a >3-fold increase in PAO activity (FIG. 4B). This significant induction of PAO message and activity in the analogue-treated human non-small cell lung carcinoma cells clearly demonstrates that PAO can be up-regulated within 24, hours in a manner similar to that observed for SSAT. Further, the induction of PAO activity correlates well with the message level, suggesting that the major regulation of PAO activity may be at the transcriptional level. This is in contrast to SSAT inductioin, where post-transcriptional regulation play a large role in the regulation of SSAT expression (19, 20).

### EXAMPLE 4

### Isoforms and Truncations of PAO

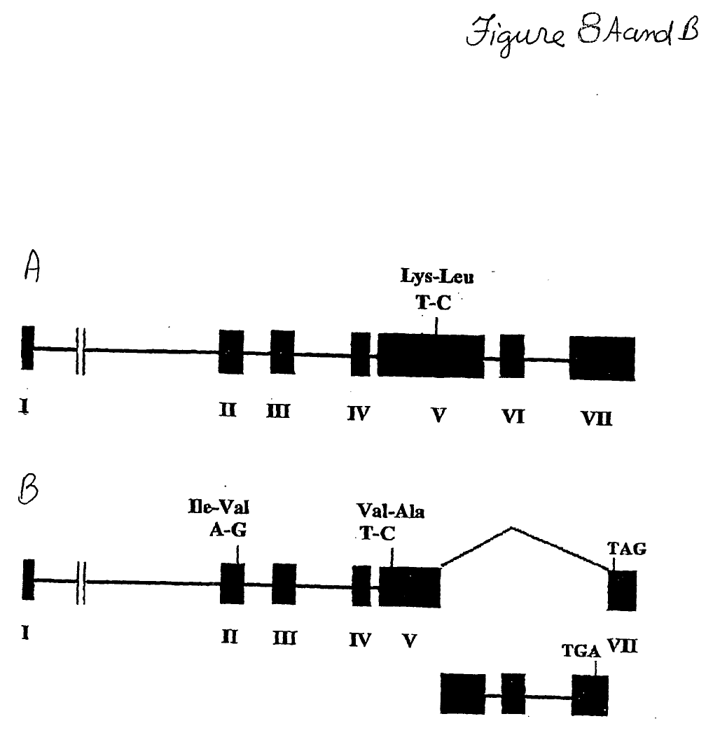

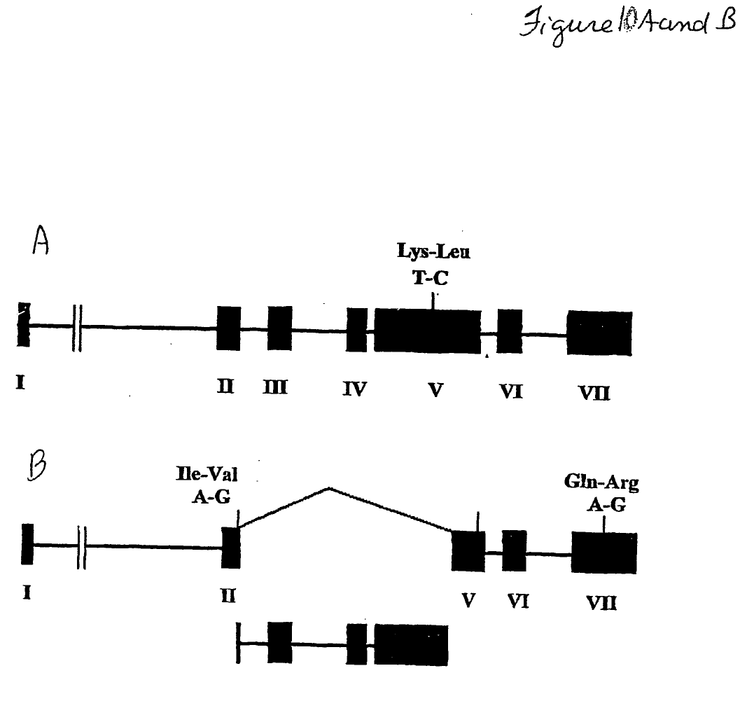

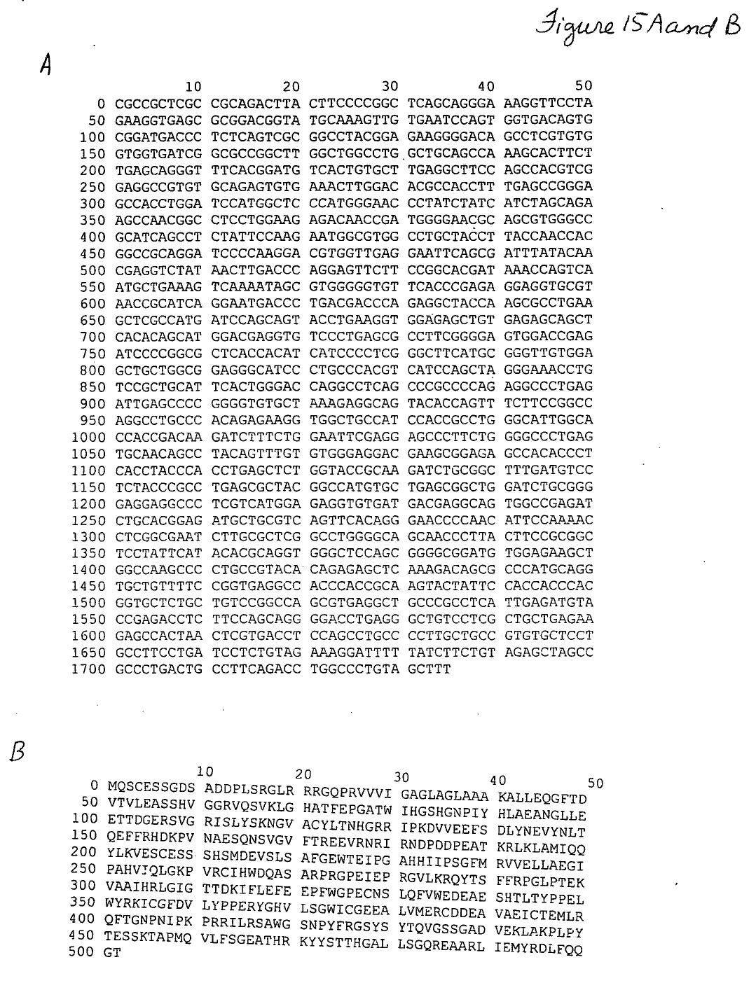

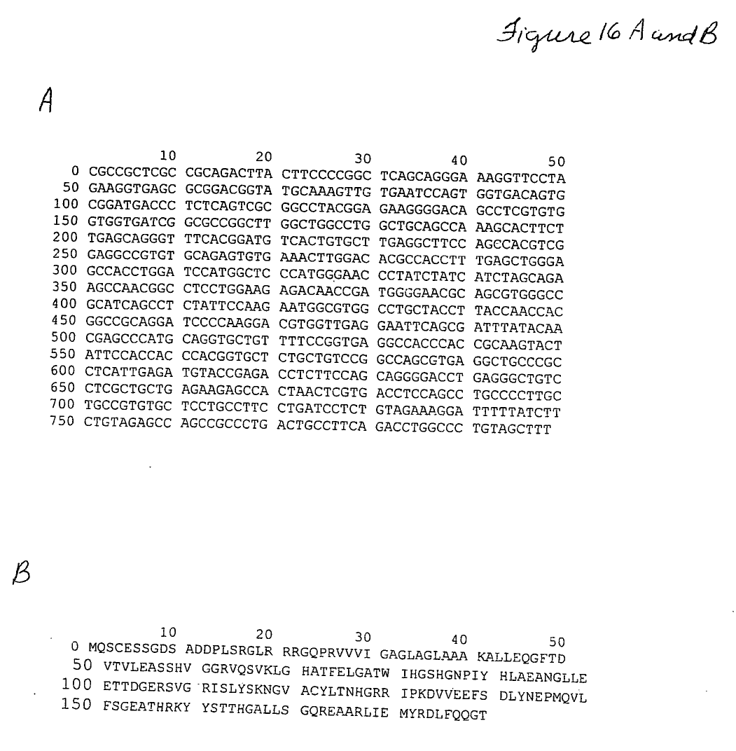

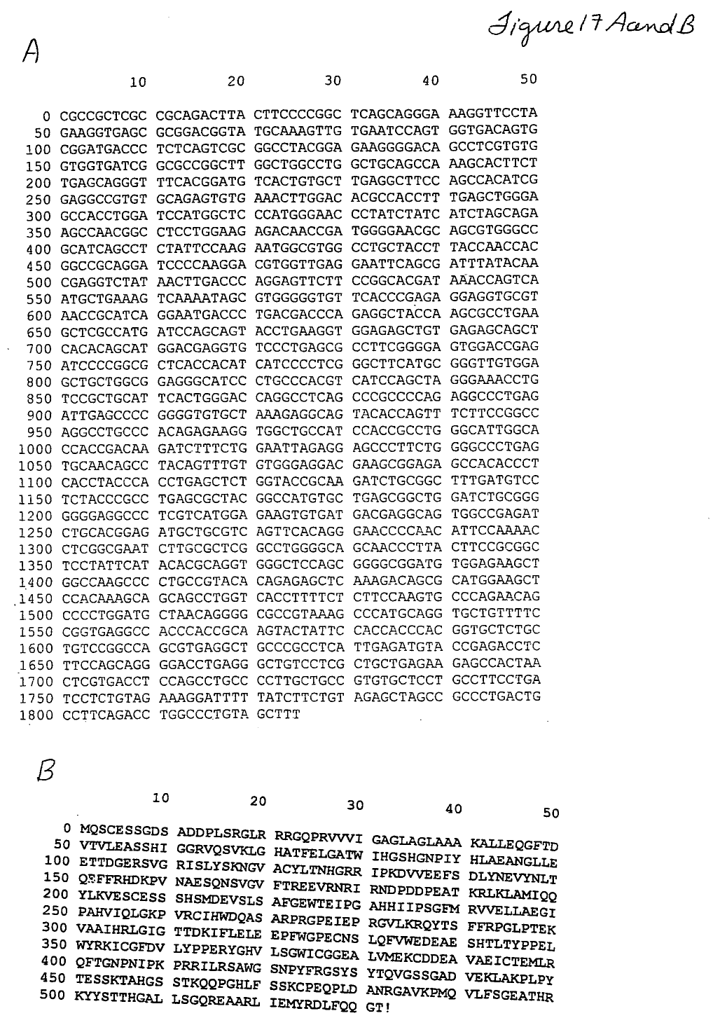

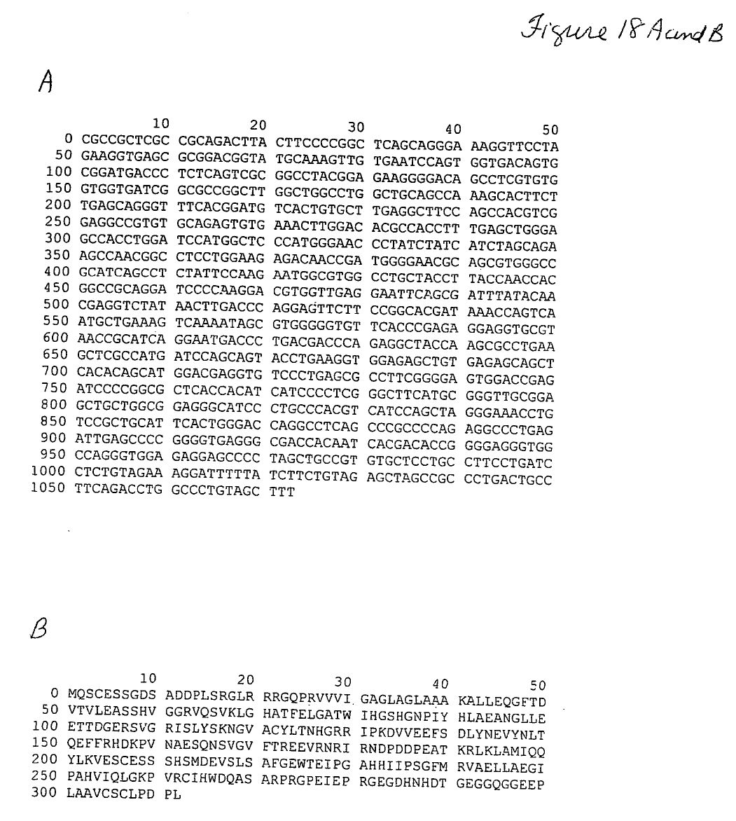

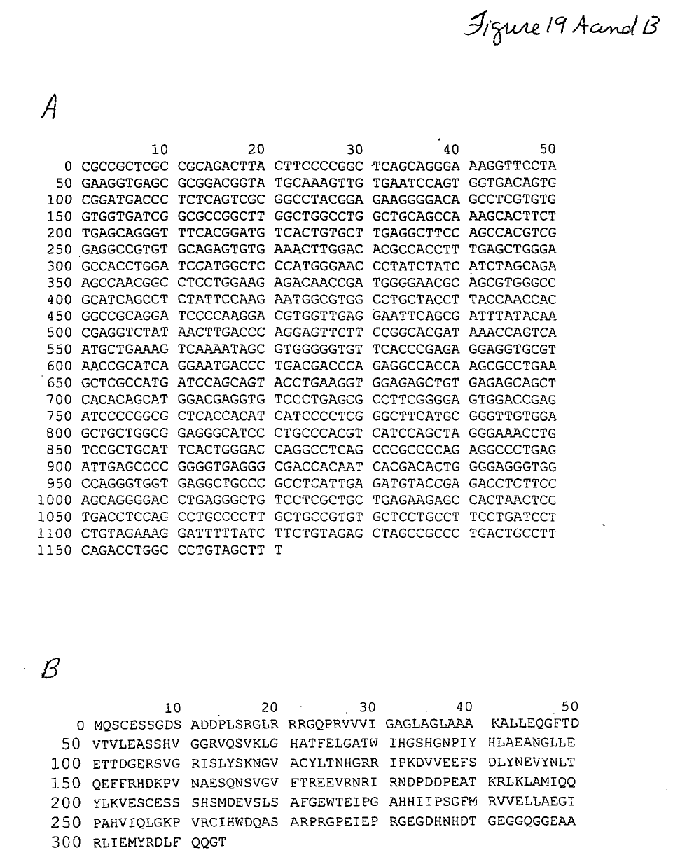

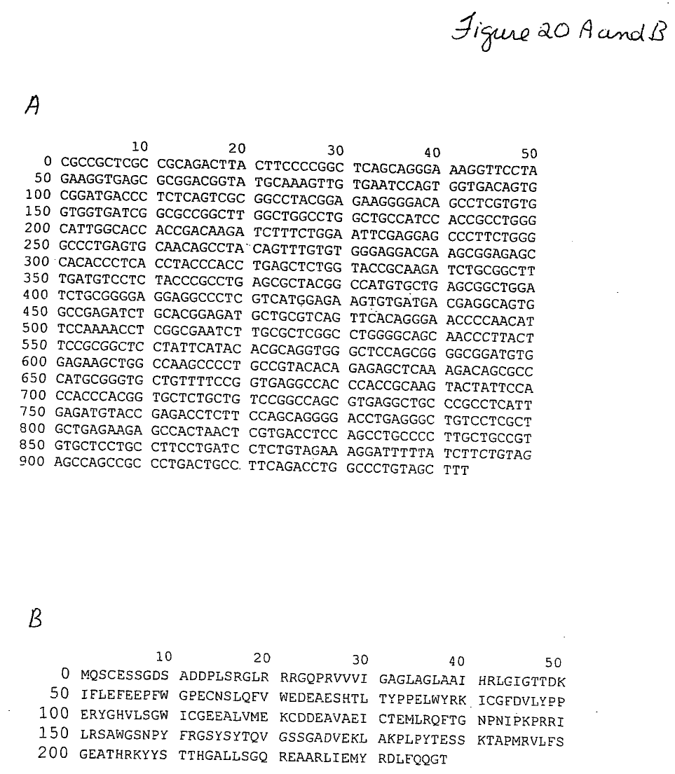

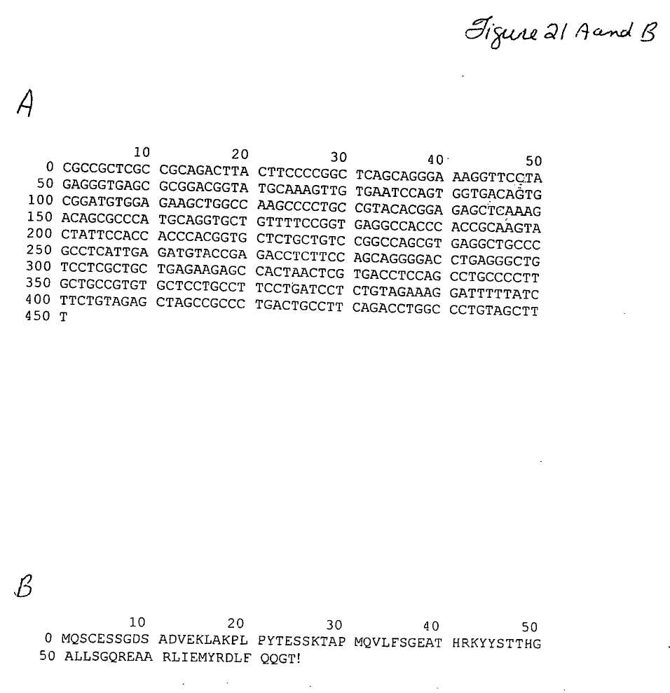

In addition to the isoform of PAO described in Examples 1-3 above, several other functional forms of mammalian PAO have been identified: three additional isoforms (Isoforms 2, 3, & 4, see FIGS. 5, 6 and 7 respectively), and four truncated (i.e. foreshortened) forms, truncations T-1, T-2, T-3 and T-4 (see FIGS. 8, 9, 10 and 11, respectively). Each of the splice variants and truncations were isolated by reverse transcriptase/polymerase chain reaction (rt-PCR) techniques. The starting source RNA for the truncations were lung tumor cell lines. The various splice variants were derived both from normal cell RNA and RNA from lung cancer cell lines. These forms are depicted schematically in FIGS. 5-11, in comparison to PAO isoform 1, and the corresponding sequences are given in figures as follows: Isoform 2, FIG. 15A and B; Isoform 3, FIG. 16A and B; Isoform 4, FIG. 17A and B; Truncation T-1, FIG. 18A and B; Truncation T-2, FIG. 19A and B; Truncation T-3, FIG. 20A and B; Truncation T-4, FIG. 21A and B. This example demonstrates that both normal and tumor tissues express a variety of PAO variants that possess different kinetic properties. These differences may have both therapeutic and disease consequences such as, but not limited to, determining the response of the cells to anticancer agents.

### EXAMPLE 5

### Induction of PAO Expression in Various Lung Cancer Cell Lines

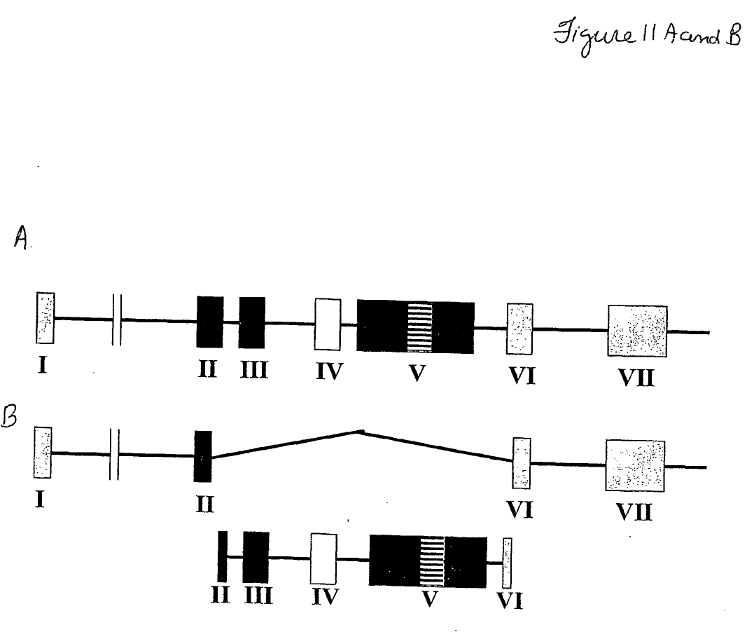

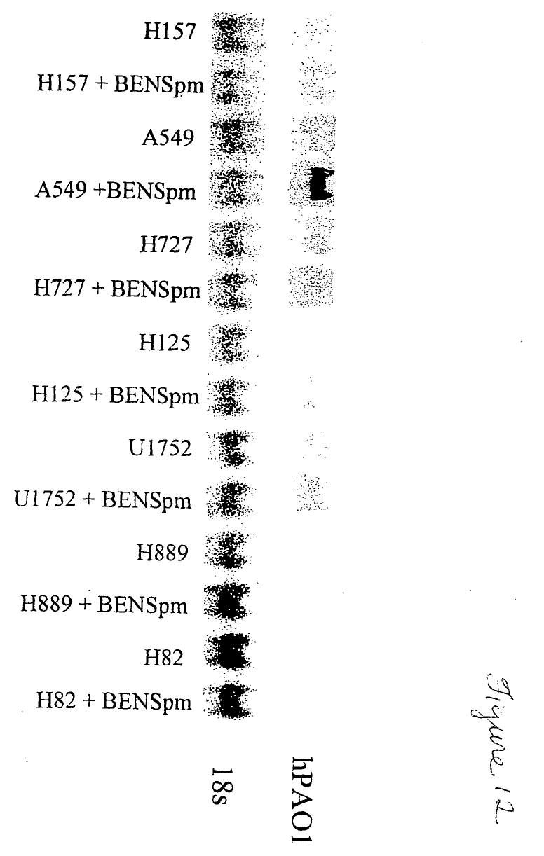

To determine if PAO expression is differentially expressed in response to exposure to the antitumor polyamine analogues cell lines representative of the major forms of human lungs were exposed to 10 μM BENSpm. The cell lines H157, H727, A549, U1752, and H125 represent non-small lung cancers, and H82 and H889 represent the small cell form of human lung cancer. Northern blot analysis was performed using 10 μg of total RNA hybridized to a radiolabeled pPAOh1 cDNA. FIG. 11 is a radiographic image of the results. FIG. 12 represents a quantitation of the results relative to the highest induced cell line, A549. It should be noted that the non-small cell lung cancers are generally more sensitive to the cell killing activity of the antitumor polyamine analogues than are the small cell lung cancer (8). This example demonstrates that the cell lines that are the most sensitive to the antitumor polyamine analogue, BENSpm, express the highest level of PAO mRNA.

### REFERENCES

1. Pegg, A. E., and McCann, P. P. Polyamine metabolism and function. Am. J. Physiol., 243: 212-221, 1982.

2. Porter, C. W., Ganis, B., Libby, P. R., and Bergeron, R. J. Correlations between polyamine analogue-induced increases in spermidine/spermine N1-acetyltransferase activity, polyamine pool depletion, and growth inhibition in human melanoma cell lines. Cancer Res., 51: 3715-3720, 1991.

3. Ha, H. C., Woster, P. M., Yager, J. D., and Casero, R. A., Jr. The role of polyamine catabolism in polyamine analogue-induced programmed cell death. Proc. Natl. Acad. Sci. USA., 94: 11557-11562,1997.

4. Marton, L. J., and Pegg, A. E. Polyamines as targets for therapeutic intervention. Annu. Rev. Pharmacol. Toxicol., 35: 55-91, 1995.

5. Bergeron, R. J., Feng, Y., Weimar, W. R., McManis, J. S., Dimova, H., Porter, C., Raisler, B., and Phanstiel, O. A comparison of structure-activity relationships between spermidine and spermine analogue antineoplastics. J. Med. Chem., 40: 1475-1494, 1997.

6. Casero, R. A., and Woster, P. M. Terminally alkylated polyamine analogues as chemotherapeutic agents. J. Med. Chem., 44: 1-26, 2001.

7. Casero, R. A., Jr., Celano, P., Ervin, S. J., Porter, C. W., Bergeron, R. J., and Libby, P. R. Differential induction of spermidine/spermine N1-acetyltransferase in human lung cancer cells by the bis(ethyl)polyamine analogues. Cancer Res., 49: 3829-3833, 1989.

8. Casero, R. A., Jr., Mank, A. R., Xiao, L., Smith, J., Bergeron, R. J., and Celano, P. Steady-state spermidine/spermine N1-acetyltransferase messenger RNA and activity correlates with sensitivity to N1, N12-bis(ethyl)spermine in human cell lines representing the major forms of lung cancer. Cancer Res., 52: 5359-5363, 1992.

9. Alhonen, L., Karppinen, A., Uusi-Oukari, M., Vujcic, S., Korhonen, V. P., Halmekyto, M., Kramer, D. L., Hines, R., Janne, J., and Porter, C. W. Correlation of polyamine and growth responses to N1, N11-diethylnorspermine in primary fetal fibroblasts derived from transgenic mice overexpressing spermidine/spermine N¹-acetyltransferase. J. Biol. Chem., 273: 1964-1969, 1998.

10. Casero, R. A., Jr., and Pegg, A. E. Spermidine/spermine N1-acetyltransferase-the turning point in polyamine metabolism. FASEB J., 7: 653-661, 1993.

11. Libby, P. R., and Porter, C. W. Separation of two isozymes of polyamine oxidase from murine L1210 leukemia cells. Biochem. Biophys. Res. Commun., 144: 528-535, 1987.

12. Seiler, N. Polyamine oxidase, properties and functions. Prog. Brain Res., 106: 333-344, 1995.

13. Chang, B. K., Liang, Y., Miller, D. W., Bergeron, R. J., Porter, C. W., and Wang, G. Effects of diethyl spermine analogues in human bladder cancer cell lines in culture. J. Urol., 150: 1293-1297, 1993.

14. Tavladoraki, P., Schinina, M. E., Cecconi, F., Di Agostino, S., Manera, F., Rea, G., Mariottini, P., Federico, R., and Angelini, R. Maize polyamine oxidase: primary structure from protein and cDNA sequencing. FEBS Lett., 426: 62-66, 1998.

15. Nishikawa, M., Hagishita, T., Yurimoto, H., Kato, N., Sakai, Y., and Hatanaka, T. Primary structure and expression of peroxisomal acetylspermidine oxidase in the methylotrophic yeast Candida boidinii. FEBS Lett., 476: 150-154, 2000.

16. Suzuki, O., Matsumoto, T., and Katsumata, Y. Determination of polyamine oxidase activities in human tissues. Experientia (Basel), 40: 838-839, 1984.

17. Sarhan, S., Quemener, V., Moulinoux, J. P., Knodgen, B., and Seiler, N. On the degradation and elimination of spermine by the vertebrate organism. Int. J. Biochem., 23: 617-626, 1991.

18. Holtta, E. Oxidation of spermidine and spermine in rat liver: purification and properties of polyamine oxidase. Biochemistry, 16: 91-100, 1977.

19. Coleman, C. S., Huang, H., and Pegg, A. E. Role of the carboxyl terminal MATEE sequence of spermidine/spermine N1-acetyltransferase in the activity and stabilization by the polyamine analog N1, N12-bis(ethyl)spermine. Biochemistry, 34: 13423-13430, 1995.

20. Coleman, C. S., and Pegg, A. E. Proteasomal degradation of spermidine/spermine N¹-acetyltransferase requires the carboxyl-terminal glutamic acid residues. J. Biol. Chem., 272: 12164-12169, 1997.

### EXAMPLE 6

### Cloning and Characterization of Multiple Human Polyamine Oxidase Splice Variants that Code for Isoenzymes with Different Biochemical Characteristics

Introduction

Further investigations of the isoforms discussed in Example 4 were carried out and are presented in this example.

Interest in polyamine catabolism has increased since its role in determining cellular sensitivity to various antitumour polyamine analogues has been acknowledged [1,2]. Until recently, the mammalian polyamine catabolic pathway was thought to consist of two enzymes, namely a rate-limiting spermidine}spermine N¹-acetyltransferase (SSAT) [3] and a polyamine oxidase (PAO) that preferentially catalyses the oxidation of the N¹-acetylpolyamines produced by SSAT activity. This oxidation results in the production of H₂O₂, 3-acetamidopropanal, and putrescine or spermidine (Spd), depending on the starting substrate [4]. H₂O₂ production by increased polyamine catabolism in response to specific polyamine analogues has been shown to result in cytotoxicity by these agents in specific tumour-cell types, and this cytotoxicity can be attenuated through the use of a specific inhibitor [5]. However, studies into the role of polyamine oxidation in mammalian cells have been limited by the lack of any verified mammalian PAO clones. Our recent cloning of the human PAO gene PAOh1 provided the first mammalian PAO clone for study and demonstrated the ability of the gene product to catalyse spermine (Spm) oxidation [6]. By using alternative methods, another group of workers have recently confirmed PAOh1 activity and have shown that, in their system, this enzyme shows a greater specificity for the native polyamine, Spm, as a substrate [7]. These data suggest that this oxidase represents an additional enzyme in polyamine catabolism that preferentially utilizes the polyamines as substrates.

The potential importance of PAOs in anticancer drug response is underscored by the finding that PAO is significantly inducible by antitumour polyamine analogues in a manner similar to SSAT [6], suggesting that its activity may play a direct role in cell death via toxic H₂O₂ production. Additionally, polyamine oxidation has recently been identified as a critical step in the detoxification of one of the antitumour polyamine analogues, and tumor cells that have low to non-detectable levels of polyamine oxidation capacity are significantly more sensitive to the cytotoxic effects of the analogue [8].

Here we report the discovery that the human PAOh1 gene codes for at least four active isoenzymes that result from alternative splicing of eight exons. The resultant proteins have different biochemical characteristics and substrate specificities, and were identified in a variety of tumour and normal cell types. Because of the potential for cell- or tissue-specific PAO isoenzyme expression levels, the products of the PAOh1 gene may contribute in unique ways to our understanding of polyamine metabolism and antitumour-drug-sensitivity.

Experimental

Abbreviations used: M-AcSpm, N¹-acetylspermine; NSCLC, non-small-cell lung carcinoma; (h1)PAO, (human) polyamine oxidase; RT-, reverse transcription; Spd, spermidine; Spm, spermine; SSAT, spermidine/spermine N¹-acetyltransferase.

Nucleotide sequences for the PAOh2, PAOh3 and PAOh4 isoforms of the human polyamine oxidase gene (PAOh) have been deposited with the GenBank2, EMBL, DDBJ and GSDB Nucleotide Sequence Databases under the accession numbers AY033890, AY033891 and AF519179 respectively.

PAO Splice Variant Cloning

PAO isoforms were isolated using reverse-transcription (RT)-PCR on total RNA from NCI-H157 non-small-cell lung carcinoma (NSCLC) cells and HEK-293 cells. A human placenta cDNA library was also used as a source for potential splice variants, as previously described [6]. PCR products were ligated into the pCR2.1 vector (Invitrogen, Carlsbad, Calif., U.S.A.), sequenced using a PerkinElmer ABI automated DNA sequencer, and analysed by comparison with the previously identified human genomic PAO sequence located in GenBank2 (accession no. AL121675). Novel sequences corresponding to alternative splicing were further cloned into the pcDNA3.1+/− mammalian expression vectors using restriction-enzyme digestion and ligation, and proper insertion was confirmed via restriction analysis. These constructs were designated pPAOh2, pPAOh3, and pPAOh4. All restriction and modification enzymes were purchased from Invitrogen or New England Biolabs (Beverly, Mass., U.S.A.).

In Vitro Transcription and Translation

For PAO splice variant protein analysis, each cDNA construct, as well as the pcDNA3.1 empty vector, was linearized using NdeI. In vitro transcription and translation was subsequently performed using the Wheat Germ Extract-TnT coupled system from Promega (Madison, Wis., U.S.A.) according to the manufacturer's protocol. Reactions were performed in parallel, with one aliquot containing [³⁵S]methionine in the amino acid mixture for subsequent separation and quantification by SDS-PAGE. Labeled reaction products were run on 10%-(w/v)-BisTris gels (Invitrogen) with 20 mM Mops buffer, pH 7.7, as suggested by the manufacturer. After drying, bands were visualized and quantified using phosphorimage analysis with Image Quant software from Molecular Dynamics (Sunnyvale, Calif., U.S.A.).

PAO Enzyme Activity Analysis

TnT products from the reactions using unlabelled substrates were used for PAO enzyme analysis by the method of Suzuki et al [9]. Specifically, 10 μl of the TnT reaction mixture was used for each 600 μl of PAO assay sample (in duplicate, and in the presence of monoamine and diamine oxidase inhibitors, as previously described [6]). Background oxidase activity in the Wheat Germ Extract-TnT reactions was determined for each substrate using the empty pcDNA3.1 vector. These values (always<0.05 pmol/min) were subtracted from the oxidase activity measured in TnT lysates produced from vectors containing the individual splice variants. To ensure linearity of the reaction (thereby determining the optimal incubation time after substrate addition), time courses were performed for each potential isoenzyme in the presence of 1 mM Spm (Sigma, St. Louis, Mo., U.S.A.).

Kinetics of PAO Isoenzymes

Apparent kinetics were examined for each isoenzyme using TnT reaction products as described above with increasing concentrations of Spm, Spd (Sigma) or N¹-acetylspermine (N¹-AcSpm; Fluka). Apparent Kₘ and Vₘₐₓ values were determined using the Lineweaver-Burk transformation of the Michaelis-Menten equation. Values were adjusted for TnT reaction efficiency by SDS-PAGE quantfication of a [³⁵S]methionine-labelled aliquot of the TnT reaction mixture normalized for methionine content.

Specific Activities of PAO Isoforms

Specific activities were determined for each isoform with each substrate at 0.25 mM. Specific activity was calculated on the basis of band intensity resulting from parallel TnT reactions in the presence of [³⁵S]methionine and normalized according to number of methionine residues present in the splice-variant protein sequences. One unit of PAO activity was defined as the ability to produce 1 pmol of H₂O₂/min per unit of protein (where 1 unit of protein corresponds to one band intensity unit, as determined from PhosphorImager analysis).

Results

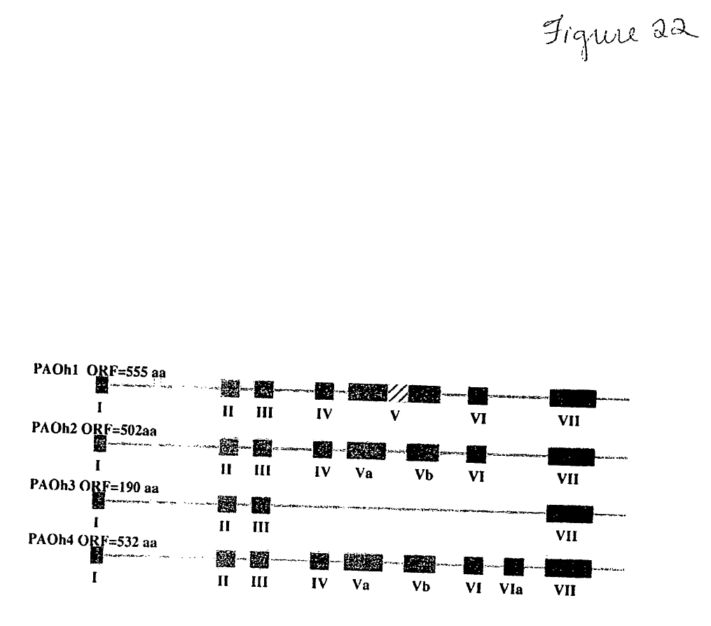

Upon sequence comparison with our previously identified PAO isoform 1 (GenBank2 accession no. AY033889), as well as with the human genomic PAO sequence (GenBank2 accession no. AL121675), we confirmed the isolation of three additional splice variants, designated PAOh2, PAOh3 and PAOh4. Isoforms 1 and 2 were isolated using the cDNA of a human placental library. PAOh2 was also isolated from the H157 NSCLC cell line, as was PAOh3, which was also obtained from HEK-293 mRNA. PAOh4 was obtained only from HEK-293 mRNA. It should be noted that exon V of PAOh1 possesses an internal region that can act as an intron, and which is spliced out of exon V of both PAOh2 and PAOh4, resulting in two smaller exons, designated exons Va and Vb. Also, PAOh4 contains an additional exon, VIa, which is not present in the other three isoforms. PAOh3 is completely devoid of exons IV-VIa, and possesses an open reading frame of only 190 amino acids, which is less than half that of the other three splice variants (FIG. 22). Surprisingly, many of the amino acids missing from PAOh3 correspond to those implicated in FAD cofactor binding in Zea mays (maize) PAO [10,11].

Since the stabilities of the various isoforms could differ, it was first necessary to determine the appropriate assay times for each splice variant to ensure measurements were made within the linear range. Time-course data using 1 mM Spm as substrate revealed a linear production of H₂O₂ by all isoforms for approximately 20 min (results not shown). Therefore 10 min was chosen as the period of incubation following substrate addition for further experiments.

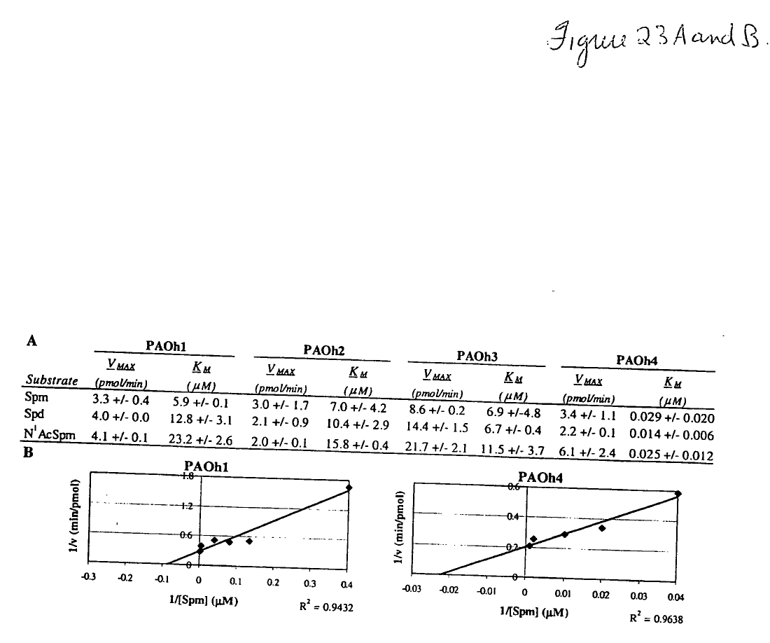

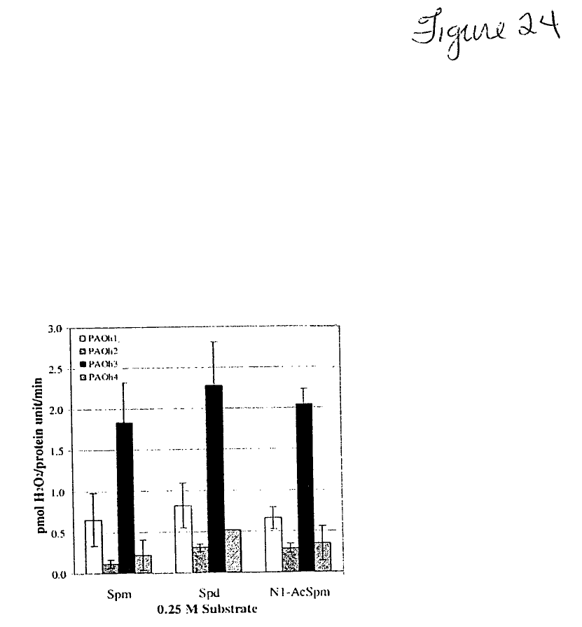

Relative kinetic analysis performed with TnT reaction products revealed distinct kinetic parameters and substrate affinities for each splice variant with Spm, Spd and N″-AcSpm (FIG. 23). Apparent Kₘ and Vₘₐₓ values for each isoform with each substrate were calculated from the Lineweaver-Burk transformation, and are presented in FIG. 23. Importantly, PAOh4 demonstrated the lowest Kₘ values for each of the substrates (in the nanomolar range), and appeared to have the highest affinity for Spd. In contrast, PAOh1 and PAOh2 had relatively low affinities for the acetylated polyamine (in the high micromolar range). The shortest of the four isoforms, PAOh3, also demonstrated a greater affinity for Spd and Spm than for N″-AcSpm. Using saturating concentrations of each substrate as apparent from FIG. 23 (0.25 mM), PAO activity assays were carried out using TnT-produced protein that was translated in parallel with an aliquot in the presence of [³⁵S]methionine. This enabled a comparison of specific activities among the splice variants based on the number of methionine residues present in the each splice variant (FIG. 24). Normalization for methionine content revealed that the shortest isoform, PAOh3, possessed the highest specific activities for all three substrates. Consistent with the predicted Kₘ and Vₘₐₓ values, Spd and N¹-AcSpm activities were slightly higher than that of Spm. PAOh1 possessed the next highest activities for all substrates, followed by PAOh2 and PAOh4, which demonstrated similar activities, in spite of the much lower Kₘ values predicted for PAOh4 (FIG. 24).

Discussion

The catabolism of polyamines has been demonstrated to be associated with the response of specific tumor types to several antitumour polyamine analogues [5,12]. Further, alterations in the enzymes that control polyamine catabolism have been implicated in the progression of neoplastic disease [13]. The mechanisms underlying these observations appear to be associated with oxidation of polyamines concurrent with the induction of SSAT. The results presented here demonstrate that each of the four identified splice variants from the PAOh1 gene are capable of catalysing the oxidation of multiple substrates, including Spd, Spm and N¹-AcSpm, suggesting that multiple oxidases coded by a single gene may significantly affect polyamine homoeostasis and potential drug response. These results are in contrast with those reported by Vujcic et al. [7], who did not obtain evidence of oxidation of any substrate other than Spm following transfection of cells with a construct homologous with PAOh1. The reasons for this variation may be a result of differences in reaction conditions. Specifically, the enzyme analyses presented here were performed in borate buffer at pH 9.0. The analyses by Vujcic et al. [7] were performed in glycine buffer at pH 9.5.

Consistent with their results, when glycine buffer was substituted for borate buffer in the system used here, only Spm was efficiently catalysed by any of the splice variants (results not shown). Another potential basis for the difference in the observations presented here from those of Vujcic et al. [7] may result from the use of a plant system (wheat-germ TnT) to produce our protein. This is necessary, since the mammalian rabbit reticulocyte system contains heme iron that would prevent the precise measurement of H₂O₂ produced [6]. The production of protein in the wheatgerm TnT may result in alternate substrate specificity based on potential differences in protein folding or a difference in cofactors or post-translational modifications between the wheat-germ system and those occurring in the transfected-cell system [14]. These results are, however, consistent with observations with the PAOs of the maize and barley (Hordeum vulgare) plants, which are nearly identical in size with the human PAOh1 protein, and which possess a protein domain organization very similar to that of the human protein. Both plant proteins are able to use both Spm and Spd as substrates [15,16]. Although the validation of substrate specificity awaits the availability of purified proteins representing the individual splice variants, the important finding that each of the splice variants codes for active proteins with different kinetic behavior is both valid and significant.

It should be noted that Vujcic et al. [7] also provided data on two constructs that were referred to as “splice variants” (GenBank2 accession nos. AK025938 and BC000669), neither of which produced oxidase activity in transfected cells. BC000669 corresponds to our PAOh2 (GenBank2 accession no. AY033890) with the exception of one amino acid at position 16. AK025938 possesses an open reading frame that codes for a polypeptide of 389 amino acids that does not correspond to any of the splice variants presented here. However, the open reading frame starts at an AUG codon corresponding to a region within exon 4 of PAOh1, and the original clone has no leader sequence associated with it. Consequently, the possibility that this cDNA represents a cloning artifact rather than an actual splice variant must be considered. More importantly, no data are provided to confirm that the transfected cells actually produce any protein from the constructs. Consequently, the studies indicating the lack of PAO activity in cells transfected with these sequences is currently difficult to interpret.

It is clear from our present results that the multiple splice variants are capable of catalysing multiple polyamine substrates in the system as reported. Interestingly, the shortest splice variant, PAOh3, appears to have the highest kcat of all the isoforms, and was the most common variant to be detected by the RT-PCR based cloning strategy used. It was isolated from the H157 and HEK-293 cells presented here, as well as in subsequent studies using normal human lymphocytes, and from DU145 prostate cancer cells. This prevalence of PAOh3 is quite possibly the result of higher cloning efficiency of the shorter sequence, but the significance that the existence of this isoform does not appear to be cell- or tissue-type-specific should not be overlooked. The Kₘ values in the micromolar range exhibited by PAOh3, as well as by PAOh1 and PAOh2, for the native polyamines is certainly within the intracellular concentration ranges often predicted, although the free intracellular polyamine concentration is difficult to estimate. PAOh4 exhibits Km values in the nanomolar range for Spd, Spm and N¹-AcSpm. This result may be significant, since the amount of acetylated polyamines in the cell is generally low, even with a high induction of SSAT activity. However, the ability of N¹-AcSpm to act as a substrate for PAOh4 in situ would depend on the concentration of free Spm and Spd, since they, too, are high-affinity substrates for PAOh4. It should be noted that PAOh4 was only obtained from the human embryonal kidney cell line.

It is not clear that any of the isoenzymes studied here is homologous with the animal PAO reported in the literature prior to our cloning of PAOh1 [4,13]. That PAO has been defined as a peroxisomal enzyme [17,18]. None of the isoenzymes presented here possesses a recognizable peroxisomal signal localization sequence [19]. Consequently, it is possible, as suggested by Vujcic et al. [7], that the isoenzymes coded for by the multiple splice variants of the PAOh1 gene represent an entirely new family of oxidases.

The results presented here demonstrate that the human PAO gene PAOh1 codes for multiple isoforms with significant activities that are capable of using multiple polyamine substrates within physiologically relevant Kₘ values. The relative expression of these various isoforms within the cell and among various cells is currently being investigated. The possibility that the various PAO isoforms may have a direct role in polyamine homoeostasis and, more importantly, in drug response to various antitumour polyamine analogues, underscores the critical importance of gaining a better understanding of this interesting group of enzymes.

References

1. Bergeron, R. J., Feng, Y., Weimar, W. R., McManis, J. S., Dimova, H., Porter, C., Raisler, B. and Phanstiel, O. (1997) A comparison of structure-activity relationships between spermidine and spermine analogue antineoplastics. J. Med. Chem. 40, 1475-1494

2. Casero, Jr, R. A. and Woster, P. M. (2001) Terminally alkylated polyamine analogues as chemotherapeutic agents. J. Med. Chem. 44, 1-26

3. Casero, Jr, R. A. and Pegg, A. E. (1993) Spermidine/spermine N¹-acetyltransferase- the turning point in polyamine metabolism. FASEB J. 7, 653-661

4. Seiler, N. (1995) Polyamine oxidase, properties and functions. Prog. Brain Res. 106, 333-344 Received 9 Oct. 2002/23 October 2002 ; accepted 24 Oct. 2002 Published as BJ Immediate Publication 24 Oct. 2002, DOI 10.1042/BJ20021587

5. Ha, H. C., Woster, P. M., Yager, J. D. and Casero, Jr, R. A. (1997) The role of polyamine catabolism in polyamine analogue-induced programmed cell death. Proc. Natl. Acad. Sci. U.S.A. 94, 11557-11562

6. Wang, Y., Devereux, W., Woster, P. M., Stewart, T. M., Hacker, A. and Casero, Jr, R. A. (2001) Cloning and characterization of a human polyamine oxidase that is inducible by polyamine analogue exposure. Cancer Res. 61, 5370-5373

7. Vujcic, S., Diegelman, P., Bacchi, C. J., Kramer, D. L. and Porter, C. W. (2002) Identification and characterization of a novel flavin-containing spermine oxidase of mammalian cell origin. Biochem. J. 367, 665-675

8. Lawson, K. R., Marek, S., Linehan, J. A., Woster, P. M., Casero, Jr, R. A., Payne, C. M. and Gerner, E. W. (2002) Detoxi/Ecation of the polyamine analogue N¹-ethyl-N¹-[(cycloheptyl)methyl]-4,8-diazaundecane (CHENSpm) by polyamine oxidase. Clin. Cancer Res. 8, 1241-1247

9. Suzuki, O., Matsumoto, T. and Katsumata, Y. (1984) Determination of polyamine oxidase activities in human tissues. Experientia 40, 838-839

10. Tavladoraki, P., Schinina, M. E., Cecconi, F., Di Agostino, S., Manera, F., Rea, G., Mariottini, P., Federico, R. and Angelini, R. (1998) Maize polyamine oxidase: primary structure from protein and cDNA sequencing. FEBS Lett. 426, 62-66

11. Binda, C., Coda, A., Angelini, R., Federico, R., Ascenzi, P. and Mattevi, A. (1999) A 30-angstrom-long U-shaped catalytic tunnel in the crystal structure of polyamine oxidase. Structure Fold. Des. 7, 265-276

12. Chen, Y., Kramer, D. L., Diegelman, P., Vujcic, S. and Porter, C. W. (2001) Apoptotic signaling in polyamine analogue-treated SK-MEL-28 human melanoma cells. Cancer Res. 61, 6437-6444

13. Wallace, H. M., Duthie, J., Evans, D. M., Lamond, S., Nicoll, K. M. and Heys, S. D. (2000) Alterations in polyamine catabolic enzymes in human breast cancer tissue. Clin. Cancer Res. 6, 3657-3661

14. Hartl, F. U. and Hayer-Hartl, M. (2002) Molecular chaperones in the cytosol: from nascent chain to folded protein. Science 295, 1852-1858

15. Cervelli, M., Cona, A., Angelini, R., Polticelli, F., Federico, R. and Mariottini, P. (2001) A barley polyamine oxidase isoform with distinct structural features and subcellular localization. Eur. J. Biochem. 268, 3816-3830

30 16. Federico, R., Alisi, C., Cona, A. and Angelini, R. (1988) Purification of polyamine oxidase from maize seedlings by immunoadsorbent column. Adv. Exp. Med. Biol. 250, 617-623

17. Van den Munckhof, R. J., Denyn, M., Tigchelaar-Gutter, W., Schipper, R. G., Verhofstad, A. A., Van Noorden, C. J. and Frederiks, W. M. (1995) In situ substrate specificity and ultrastructural localization of polyamine oxidase activity in unfixed rat tissues. J. Histochem. Cytochem. 43, 1155-1162

18. Nishikawa, M., Hagishita, T., Yurimoto, H., Kato, N., Sakai, Y. and Hatanaka, T. (2000) Primary structure and expression of peroxisomal acetylspermidine oxidase in the methylotrophic yeast Candida boidinii. FEBS Lett. 476, 150-154

19. Holroyd, C. and Erdmann, R. (2001) Protein translocation machineries of peroxisomes. FEBS Lett. 501, 6-10

### EXAMPLE 7

### Induction of the PAOh1/SMO Polyamine Oxidase by Polyamine Analogues in Human Lung Carcinoma Cells

Expanded investigations of induction of PAOh1/SNO polyamine oxidase by polyamine analogues in human lung carcinoma cells, as discussed in Example 5, were carried out and are presented in this example.

Introduction

Based on the ubiquitous requirement of tumor cell growth for polyamines, polyamine metabolism has been intensely investigated as a target for antineoplastic therapy [7, 26]. Although much work has focused on inhibiting the biosynthesis of polyamines through the rate-limiting biosynthetic enzymes ornithine decarboxylase and S-adenosylmethionine [30], the development of several antitumor polyamine analogues has resulted in increased interest in the regulation of polyamine catabolism [1, 2, 6, 7, 8, 17, 19, 33]. The interest in polyamine catabolism increased with the discovery that spermidine/spermine N¹-acetyltransferase (SSAT), a rate-limiting step in polyamine catabolism, is associated with the cell type-specific cytotoxic activity of some polyamine analogues [8, 10, 27, 31]. The increase in SSAT activity produces a rapid increase in acetylated polyamines which then become substrates of an acetylpolyamine oxidase (PAO) [36, 41]. It was originally hypothesized that H₂O₂ production from this last step in the two-step polyamine catabolism was responsible for cytotoxicity in some cases [12, 20]. However, our recent cloning of a new polyamine oxidase (PAOh1/SMO) that can efficiently use spermine as a substrate, and is inducible by polyamine analogues, has revealed a new and potentially exploitable target in polyamine metabolism [42]. Similar to the activity of PAO, PAOh1/SMO results in the production of H₂O₂, a reactive oxygen species that has been directly linked with the phenotype-specific toxicity of specific antitumor polyamine analogues [36]. In this study the induction of PAOh1/SMO by multiple antitumor polyamine analogues was examined in multiple lung cancer cell types representative of the various phenotypes of human lung cancer to demonstrate that PAOh1/SMO induction, similar to the induction of SSAT, is a phenotype-specific response to the analogues [8, 9, 10, 31, 32, 37]. The results also suggest that, similar to the effects observed with the high induction of SSAT, the high induction of PAOh1/SMO may also be linked with analogue-induced cytotoxicity for specific analogues.

Materials and Methods

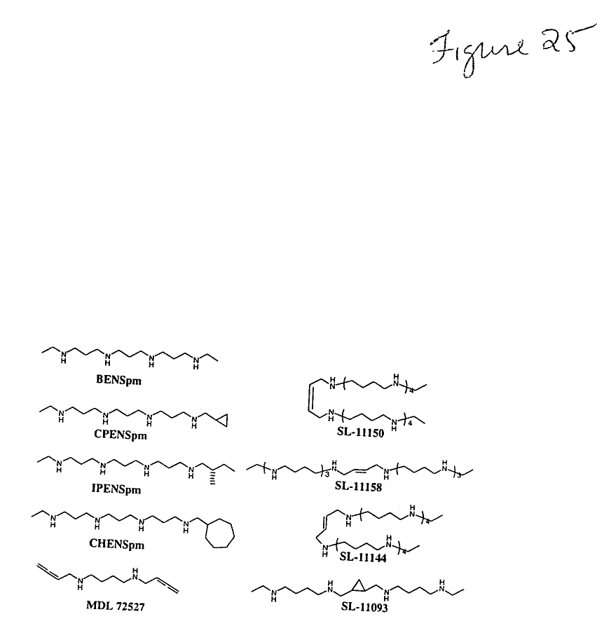

Abbreviations: BENSpm N1,N11-bis(ethyl)norspermine; CHENSpm N1-ethyl-N11-(cycloheptyl)methyl-4,8, diazaundecane; CPENSpm N1-ethyl-N11-(cyclopropyl) methyl-4,8,diazaundecane; DAO Diamine oxidase; IPENSpm (S)-N1-(2-methyl-1-butyl)-N11-ethyl-4,8, diazaundecane; MAO Monoamine oxidase; MDL 72,527 (N1,N4-bis(2,3-butadienyl)-1,4-butanediamine); PAO Acetylpolyamine oxidase; PAOh1/SMO Human polyamine oxidase h1/spermine oxidase; SSAT Spermidine/spermine N1-acetyltransferase Chemicals and reagents: N¹,N¹¹-bis(ethyl)norspermine (BENSpm) was provided by Parke-Davis (Ann Arbor, Mich.) and N¹-ethyl-N¹¹-(cyclopropyl) methyl-4,8,diazaundecane (CPENSpm), N¹-ethyl-N¹¹-(cycloheptyl) methyl-4,8,diazaundecane (CHENSpm), (S)-N1-(2-methyl-1-butyl)-N¹¹-ethyl-4,8,diazaundecane (IPENSpm), SL-11150, SL-11158, SL-11144, and SL-11093, and the selective PAOh1/SMO inhibitor MDL 72,527 were synthesized as previously reported [3, 4, 34, 35, 39, 44] (FIG. 25). N1-Acetylspermine was purchased from Fluka (Buchs, Switzerland). Stock solutions (10 mM) of the various analogues were prepared in ddH₂O and stored at )20° C. Other chemicals were obtained from Sigma Chemical Company (St. Louis, Mo.), Invitrogen/Life Technologies (Rockville, Md.), Bio-Rad (Hercules, Calif.), Aldrich Chemical Company (Milwaukee, Wis.), Hyclone (Logan, Utah), and J. T. Baker (Phillipsburg, N.J.).

Cell Culture and Analysis of Growth Inhibition of Human Lung Cancer Cell Lines The non-small-cell cancer lines NCI A549 (adenocarcinoma), NCI H157 (squamous), NCI H727 (carcinoid), NCI H125 (adenocarcinoma) and U1752 (squamous), and the small-cell lung carcinoma lines NCI H82 and NCI H889 were cultured as we have previously described [10, 25]. Cells were treated for the times and with the concentrations of specific agents as indicated in the Results. For cell growth analysis in the presence of the PAOh1/SMO inhibitor MDL 72,527, the MTS dye reduction assay CellTiter 96 system from Promega was used according to the supplier's protocol. In these experiments, NCI A549 cells were seeded at 5×10³ cells/well in a 96-well microtiter plate and treated for 96 h with 10 μM of the indicated analogue in the presence or absence of 250 μM MDL 72,527.

RNA Purification and Northern Blot Analysis

Total cellular RNA was extracted from the lung cancer cell lines using Trizol reagent (Invitrogen) according to the manufacturer's protocol. For Northern blotting, total RNA (20 lg) was separated on a denaturing 1.5% agarose gel containing 6% formaldehyde and transferred to Zetaprobe membrane (Bio-Rad). Random primer- labeled PAOh1 cDNA was used as probe to estimate PAOh1/SMO expression [42]. Blots were stripped and reprobed with an 18S ribosomal cDNA to provide a loading control. Analysis of polyamine content, SSAT and PAOh1/SMO activity. Intracellular polyamine concentrations were determined using the precolumn dansylation labeling, reverse-phase high-pressure liquid chromatography method as described by Kabra et al. [22] using 1,7-diaminoheptane as an internal standard. Polyamine concentrations are reported as nanomoles per milligram protein for each sample, where lysate protein content was measured by the method of Bradford [5]. SSAT activity of cellular extracts was measured as previously described [8]. The PAOh1/SMO enzyme activity in the cell lysates was assayed as previously described [42] by the method of Suzuki et al. [38] using 250 μM spermine as the substrate. The PAOh1/SMO assays were performed in the presence of 1.0 mM pargyline and 0.1 mM semicarbizide as inhibitors of monoamine oxidase (MAO) and diamine oxidase (DAO), respectively.

Results

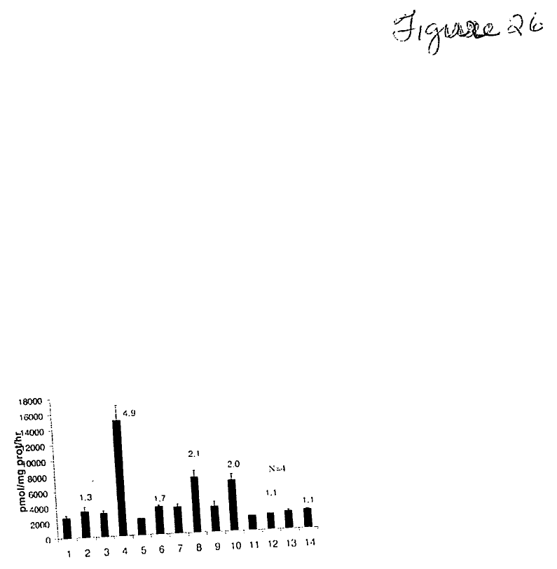

PAOh1/SMO expression in human lung cancer cell lines in response to BENSpm exposure BENSpm was chosen for the majority of studies reported here because it is one of the polyamine analogues that has been examined clinically [21] and because our initial studies indicated that PAOh1/SMO mRNA and PAOh1/SMO activity increases in a non-small-cell lung cancer line after 24 h exposure to 10 1M BENSpm [42]. Therefore we examined the ability of BENSpm to induce PAOh1/SMO in seven human lung cancer cell lines that represent the major phenotypes of lung cancer (FIG. 26). Five cell lines exhibited modest to significant induction of PAOh1/SMO activity (FIG. 26), with the adenocarcinoma line NCI A549 exhibiting the highest fold induction of PAOh1/SMO activity (about fivefold). It should be noted that the basal levels of oxidase activity do not directly reflect the amount of specific PAOh1/SMO activity since less than 10% of the basal level is inhibited by MDL 72,527 (see below). Consequently, the fold-induction estimates most probably underestimate the actual fold-induction. The observed increases in PAOh1/SMO activity in the individual cell types were reflected by similar increases in steady-state PAOh1/SMO mRNA levels (not shown). There was no observed increase in PAOh1/SMO activity or mRNA in the two small-cell lung cancer lines examined.

Time- and Dose-Dependency of Analogue-Induced PAOh1/SMO Activity

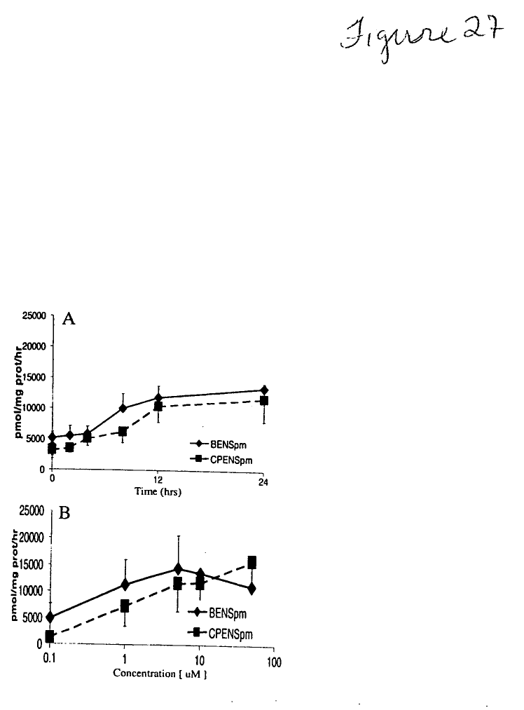

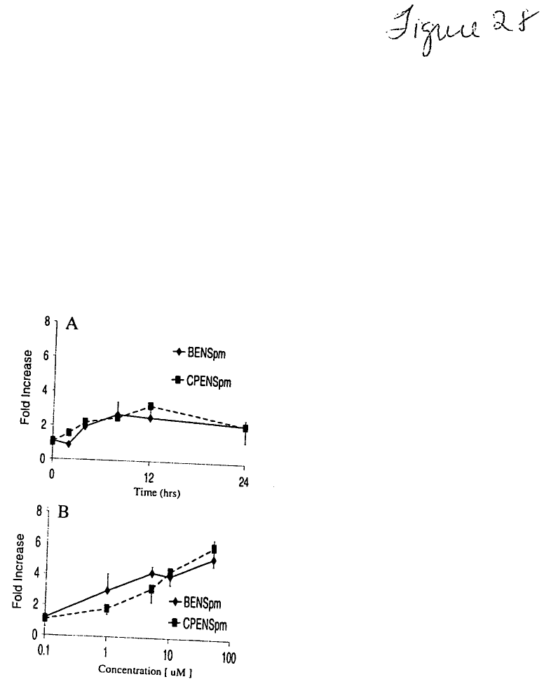

Since the NCI A549 cell line demonstrated the largest induction of PAOh1/SMO activity in response to BENSpm treatment, this line was chosen for further testing. CPENSpm treatment was also performed since it has also been shown to induce SSAT in a manner similar to BENSpm in these cells [11]. The effects of increasing time of BENSpm exposure on PAOh1/SMO activity were readily observed (FIG. 27A) when cells were exposed to 10 μM BENSpm for 0.5 to 24 h. The activity had increased approximately threefold by 12 h and nearly fivefold by 24 h. When NCI A549 cells were exposed to increasing concentrations of BENSpm for 24 h, the maximal induction of PAOh1/SMO was observed at 5 μM with lower activity at higher concentrations (FIG. 27B). Nearly identical results were observed when cells were exposed to the polyamine analogue CPENSpm, with the exception that PAOh1/SMO activity continued to increase at concentrations up to 50 μM CPENSpm (FIG. 27). The increase in PAOh1/SMO activity was generally accompanied by an increase in the 2.4 kb steady-state PAOh1/SMO mRNA (FIG. 28). It should be noted that the NCI A549cells readily accumulated both BENSpm and CPENSpm, resulting in a decrease in intracellular polyamine pools and an induction of SSAT activity (Table 1). We have previously demonstrated that both compounds are cytotoxic to non-small-cell lung cancer lines after 96 h exposure to concentrations >1 μM [7, 11].

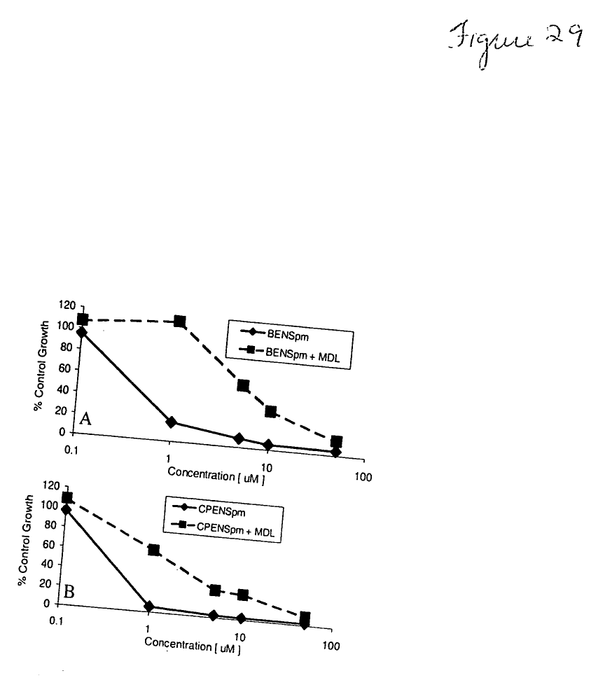

To determine whether the inhibition of PAOh1/SMO activity could alter the response of NCI A549 cells to BENSpm or CPENSpm, cell growth studies were performed where the cells were simultaneously exposed to the analogue and the PAOh1/SMO inhibitor MDL 72,527 and cultured for 96 h (FIG. 29). There was a clear decrease in sensitivity to both analogues in the presence of the PAOh1/SMO inhibitor.

Substrate Specificity of Analogue-Induced PAOh1/SMO in NCI A549 Cells

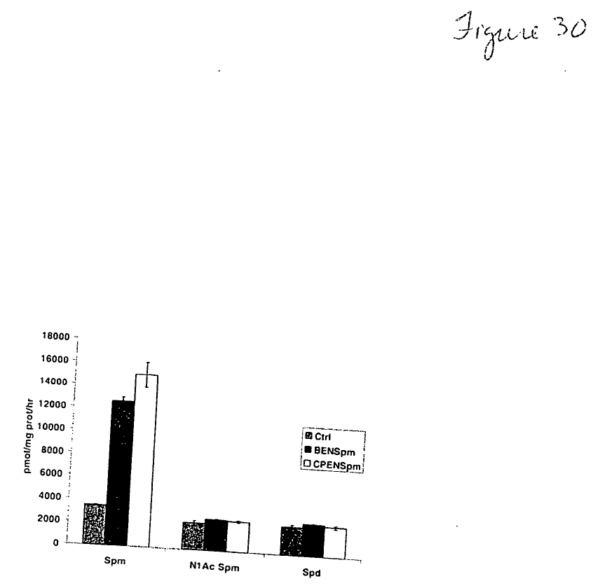

Vujcic et al. have recently reported that in a transfection system using an expression vector containing virtually the identical sequence we originally identified as PAOh1, the resulting lysate could only e.ciently use spermine as a substrate [40]. To determine the substrate specificity of the PAOh1/SMO activity induced in the NCI A549 cells, cell lysates from cells treated with either 10 μM BENSpm or 10 μM CPENSpm for 24 h were examined for their ability to catalyze spermine, spermidine, and N¹-acetylspermine (FIG. 30). This concentration was chosen since it has previously been demonstrated to significantly induce polyamine catabolism through SSAT and because it is an attainable concentration of BENSpm in the clinical setting [9, 21]. The results clearly indicated that only spermine was a substrate for the inducible PAOh1/SMO activity in the NCI A549 lysates.

Induction of PAOh1/SMO by Polyamine Analogues is Structure Dependent

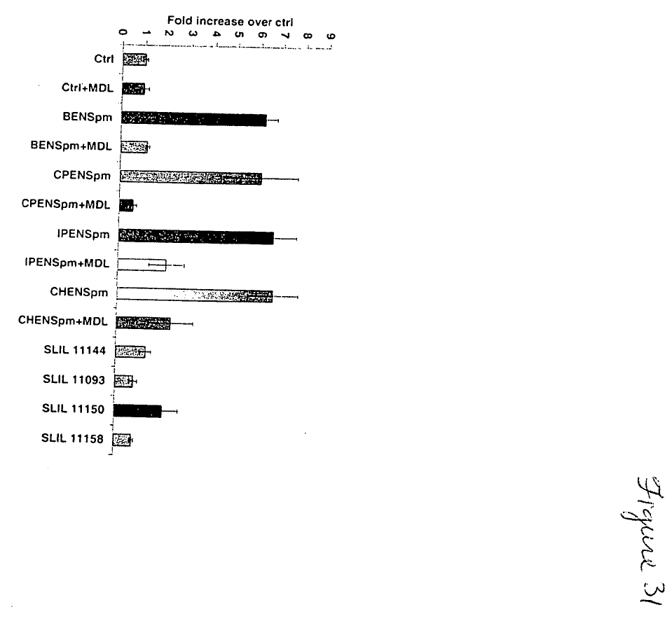

To determine the basic structural requirements of PAOh1/SMO induction in NCI A549 cells, the ability of eight polyamine analogues that are undergoing or are being considered for clinical trials were examined for their ability to induce PAOh1/SMO activity. The symmetrically substituted BENSpm, and the asymmetrically substituted CPENSpm, CHENSpm, and IPENSpm led to significant induction of PAOh1/SMO after 24 h exposure to 10 μM of each analogue (FIG. 31). The oligoamine analogues, SLIL 11144, 11150, 11158, and the conformationally restricted analogue, SLIL 11093, did not induce PAOh1/SMO. The results of these studies suggest that one structural requirement for PAOh1/SMO induction is the presence of multiple aminopropyl moieties within the analogue structure. It is also important to note that the polyamine oxidase inhibitor MDL 72,527 significantly inhibited the PAOh1/SMO activity induced by the analogues, but did not significantly reduce the basal levels of oxidase activity. These data are consistent with the possibility that there is a basal oxidase activity in the NCI A549 cells that is not inducible by polyamine analogues and is not inhibited by PAOh1/SMO, MAO, or DAO inhibitors.

TABLE 1
Effects of 24-h analogue treatment of NCI A549 cells on polyamine pools,
PAOh1/SMO and SSAT activities.

| | | | | | | SSAT (pmol |
|---|---|---|---|---|---|---|
| | | | | Analogue | PAOh1/SMO | N¹-acetyl- |
| | Polyamine (nmol/mg protein) | | | (nmol/mg | (pmol H₂O₂/ | spermidine/mg |
| Treatment | Putrescine | Spermidine | Spermine | protein) | mg/h) | protein/min) |
| None | 2.1 ± 0.7 | 26.9 ± 1.2 | 9.9 ± 1.2 | N/A | 6,390 ± 2,070 | 5 ± 0.1 |
| 10 μM | <0.05 | 1.2 ± 0.8 | 1.2 ± 0.3 | 49.4 ± 9.8 | 16,100 ± 780 | 5590 ± 517.7 |
| BENSpm | | | | | | |
| 10 μM | <0.05 | 5.5 ± 0.2 | 2.4 ± 0.4 | 17.8 ± 1.4 | 14,300 ± 3,700 | 1190 ± 287.9 |
| CPENSpm | | | | | | |

Values are means ± SE, four experiments in duplicate for the polyamines, and four experiments in triplicate for PAOh1/SMO and SSAT (SSAT spermidine/spermine N1-acetyltransferase)

Discussion

The role of polyamine catabolism in response to antitumor polyamine analogues and other agents has become an intense area of investigation based on the discovery that it is, in some instances, causally associated with the cell type-specific cytotoxicity of these agents [12, 20, 24, 29]. However, the study of polyamine catabolism in mammalian cells had previously been limited since no mammalian polyamine oxidases had been cloned. Our laboratory helped fill this deficiency by providing the first clone of a human polyamine oxidase (PAOh1) that could readily use spermine as a substrate [42]. Here data are presented which demonstrate that this newly characterized enzyme is inducible in a tumor cell type-specific and agent-specific manner. These results suggest that PAOh1/SMO activity can have an effect on tumor cell response to specific antitumor polyamine analogues. Similar to results observed with analogue-induction of SSAT, the non-small-cell lung cancer phenotypes responded to analogue exposure with a higher induction of PAOh1/SMO than did the small-cell lung cancer phenotypes. However, it should be noted that unlike SSAT, which is most highly expressed in the non-small cell lung cancer line NCI H157, PAOh1/SMO was found to be most highly expressed in the adenocarcinoma cell line NCI A549, at the levels of both mRNA (FIG. 26) and activity [10]. The results indicating that PAOh1/SMO is an inducible enzyme are significant since previous data suggest that the oxidation of polyamines is limited by the availability of acetylated substrate [36]. However, in the NCI A549 cells the inducible PAOh1/SMO activity clearly preferred spermine as the substrate, and was not significantly active on either N1-acetylspermine or spermidine (FIG. 30). Additionally, preliminary studies demonstrated that induction of PAOh1/SMO activity by the polyamine analogues occurs in a phenotype-specific manner in a number of human tumor cell types including breast, prostate and colon cancer cell lines [16]. These results are consistent with those recently reported by Vujcic et al. [40] who used a transfection model to demonstrate a spermine preference by an essentially identical clone (identified as SMO by Vujcic et al.) to the PAOh1h1 clone we originally reported. It is important to note that during the preparation of this report, Vujcic et al. [41] reported the identification of a mammalian oxidase that preferentially oxidizes acetylated polyamines. It should also be stressed that multiple splice variants of PAOh1 have been identified and each appears to possess different kinetic properties [28]. However, more study is necessary to determine the spectrum of expression of these splice variants in normal and tumor cells in order to determine the extent of their physiological relevance. Each of the key enzymes in polyamine metabolism has been demonstrated to be regulated at multiple levels [26, 30]. SSAT is known to be significantly post-transcriptionally regulated. The levels of SSAT protein induced by polyamine analogues are often in excess of those expected by observed increases in SSAT mRNA [13, 14, 15, 18]. Interestingly, in the case of PAOh1/SMO, the increase in steady-state PAOh1/SMO mRNA closely parallels the observed increase in PAOh1/SMO activity. This parallel increase in message and activity was seen in both the time- and dose-dependent studies.

These results suggest that PAOh1/SMO may be primarily regulated by changes in mRNA levels; however, formal transcriptional studies will have to be performed to determine if transcription is the key regulatory step. The induction of PAOh1/SMO in A549 cells appears to be agent-specific. Interestingly, the agents that were demonstrated to be the best inducers of SSAT (BENSpm, CPENSpm, etc.) also, with one exception, appear to be the best inducers of PAOh1/SMO [7]. The one exception is CHENSpm, which did not significantly induce SSAT in most cell types, but clearly was an effective inducer of PAOh1/SMO in the A549 cells. It should be noted that CHENSpm has been shown to be a substrate of a polyamine oxidase activity in CHO cells [23]. However, it is likely that this oxidase activity is the classical acetylpolyamine oxidase PAO recently cloned by Vujcic et al. [40] and not PAOh1/SMO activity. Importantly, Wang et al. [43] have demonstrated that none of the analogues used in this study is an effective substrate for purified PAOh1/SMO.

The small number of conformationally restricted and oligoamine analogues used in this study did not demonstrate an ability to induce PAOh1/SMO. Based on these data, it appears that the presence of three-carbon bridges between nitrogens as exist in BENSpm and CPENSpm are critical to PAOh1/SMO induction. These results, although derived from only a small number of analogues, are significant since there is increasing interest in the development of antitumor polyamine analogues and because the oxidation of polyamines can play a significant role in determining the relative sensitivity of particular tumor types to the analogues. This role is particularly evident in the studies demonstrating that the inhibition of PAOh1/SMO activity by MDL 72,527 significantly alters the dose response of NCI A549 in response to both BENSpm and CPENSpm requiring greater concentration of analogue to produce the growth effects. However, it is important to note that the concentration of MDL 72,527 used here did not adversely affect the growth of NCI A549 cells. It should also be emphasized that these oligoamines and conformationally restricted analogues have demonstrated significant in vitro activity against human prostate and breast cancer cells [34, 35] and in our lung cancer cell panel (unpublished observations). Therefore, PAOh1/SMO induction may be one component of specific analogue toxicity, but it is clearly not a requirement for all analogue activity.

Polyamine metabolism continues to be a focus of promising antineoplastic drug development. The recent discovery of a new enzyme in the polyamine catabolic pathway that produces H₂O₂, a mediator of toxicity that has been directly linked to tumor cell response, provides yet another potentially exploitable target. Although further study is required to determine the full potential of drug-induced modulation of PAOh1/SMO, the current results present an important start in understanding the role of PAOh1/SMO in de.ning tumor sensitivity to specific agents.

References

1. Bergeron R J, Neims A H, McManis J S, Hawthorne T R, Vinson J R, Bortell R, Ingeno M J (1988) Synthetic polyamine analogues as antineoplastics. J Med Chem 31:1183

2. Bergeron R J, Feng Y, Weimar W R, McManis J S, Dimova H, Porter C, Raisler B, Phanstiel O (1997) A comparison of structure-activity relationships between spermidine and spermine analogue antineoplastics. J Med Chem 40:1475

3. Bey P, Bolkenius F N, Seiler N, Casara P (1985) N-2,3-Butadienyl-1,4-butanediamine derivatives: potent irreversible inactivators of mammalian polyamine oxidase. J Med Chem 28:1

4. Bolkenius F N, Bey P, Seiler N (1985) Specific inhibition of polyamine oxidase in vivo is a method for the elucidation of its physiological role. Biochim Biophys Acta 838:69

5. Bradford M M (1976) A rapid and sensitive method for the quantitation of microgram quantities of protein utilizing the principle of protein-dye binding. Anal Biochem 72:248

6. Casero R A, Pegg A E (1993) Spermidine/spermine N1-acetyltransferase—the turning point in polyamine metabolism. FASEB J 7:653

7. Casero R A, Woster P M (2001) Terminally alkylated polyamine analogues as chemotherapeutic agents. J Med Chem 44:1

8. Casero R A, Celano P, Ervin S J, Porter C W, Bergeron R J, Libby P R (1989) Di.erential induction of spermidine/spermine N¹-acetyltransferase in human lung cancer cells by the bis(ethyl)polyamine analogues. Cancer Res 49:3829

9. Casero R A, Celano P, Ervin S J, Wiest L, Pegg A E (1990) High specific induction of spermidine/spermine N1-acetyltransferase in a human large cell lung carcinoma. Biochem J 270:615

10. Casero R A, Mank A R, Xiao L, Smith J, Bergeron R J, Celano P (1992) Steady-state messenger RNA and activity correlates with sensitivity to N¹,N¹²-bis(ethyl)spermine in human cell lines representing the major forms of lung cancer. Cancer Res 52:5359

11. Casero R A, Mank A R, Saab N H, Wu R, Dyer W J, Woster P M (1995) Growth and biochemical e.ects of unsymmetrically substituted polyamine analogues in human lung tumor cells. Cancer Chemother Pharmacol 36:69

12. Chen Y, Kramer D L, Diegelman P, Vujcic S, Porter C W (2001) Apoptotic signaling in polyamine analogue-treated SK-MEL-28 human melanoma cells. Cancer Res 61:6437

13. Coleman C S, Pegg A E (1997) Proteasomal degradation of spermidine/spermine N1-acetyltransferase requires the carboxyl-terminal glutamic acid residues. J Biol Chem 272:12164

14. Coleman C S, Pegg A E (2001) Polyamine analogues inhibit the ubiquitination of spermidine/spermine N1-acetyltransferase and prevent its targeting to the proteasome for degradation. Biochem J 358:137

15. Coleman C S, Huang H, Pegg A E (1995) Role of the carboxyl terminal matee sequence of spermidine/spermine N1-acetyltransferase in the activity and stabilization by the polyamine analog N1,N12-bis(ethyl)spermine. Biochemistry 34:13423

16. Devereux W, Wang Y, Murray Stewart T, Hacker A, Smith R, Ward T D, Woster P A, Casero R A (2003) Induction of polyamine oxidase by polyamine analogues in multiple human tumor lines. Proc Am Assoc Cancer Res 44:831

17. Fogel-Petrovic M, Vujcic S, Brown P J, Haddox M K, Porter C W (1996) Effects of polyamines, polyamine analogs, and inhibitors of protein synthesis on spermidine-spermine N¹-acetyltransferase gene expression. Biochemistry 35:14436

18. Fogel-Petrovic M, Vujcic S, Miller J, Porter C W (1996) Differential post-transcriptional control of ornithine decarboxylase and spermidine-spermine N1-acetyltransferase by polyamines. FEBS Lett 391:89

19. Fogel-Petrovic M, Kramer D L, Vujcic S, Miller J, McManis J S, Bergeron R J, Porter C W (1997) Structural basis for differential induction of spermidine/spermine N1-acetyltransferase activity by novel spermine analogs. Mol Pharmacol 52:69

20. Ha H C, Woster P M, Yager J D, Casero R A (1997) The role of polyamine catabolism in polyamine analogue-induced programmed cell death. Proc Natl Acad Sci USA 94:11557

21. Hahm H A, Ettinger D S, Bowling K, Hoker B, Chen T L, Zabelina Y, Casero R A (2002) Phase I study of N1,N11-diethylnorspermine in patients with non-small cell lung cancer. Clin Cancer Res 8:684

22. Kabra P M, Lee H K, Lubich W P, Marton L J (1986) Solidphase extraction and determination of dansyl derivatives of unconjugated and acetylated polyamines by reversed-phase liquid chromatography: improved separation systems for polyamines in cerebrospinal .uid, urine and tissue. J Chromatogr 380:19

23. Lawson K R, Marek S, Linehan J A, Woster P M, Casero R A, Payne C M, Gerner E W (2002) Detoxification of the polyamine analogue N1-ethyl-N11-[(cycloheptyl)methyl]-4,8-diazaundecane (Chenspm) by polyamine oxidase. Clin Cancer Res 8:1241

24. Lindsay G S, Wallace H M (1999) Changes in polyamine catabolism in Hl-60 human promyelogenous leukaemic cells in response to etoposide-induced apoptosis. Biochem J 337:83

25. Mank-Seymour A R, Murray T R, Berkey K A, Xiao L, Kern S, Casero R A (1998) Two active copies of the X-linked gene spermidine/spermine N1-acetyltransferase (Ssat) in a female lung cancer cell line are associated with an increase in sensitivity to an antitumor polyamine analogue. Clin Cancer Res 4:2003

26. Marton L J, Pegg A E (1995) Polyamines as targets for therapeutic intervention. Annu Rev Pharmacol Toxicol 35:55

27. McCloskey D E, Pegg A E (2000) Altered spermidine/spermine N¹-acetyltransferase activity as a mechanism of cellular resistance to bis(ethyl)polyamine analogues. J Biol Chem 275:28708

28. Murray Stewart T, Wang Y, Devereux W, Casero R. A. (2002) Cloning and characterization of human polyamine oxidase splice variants. Proc Am Assoc Cancer Res 43:1120

29. Niiranen K, Pietila M, Pirttila T J, Jarvinen A, Halmekyto M, Korhonen V P, Keinanen T A, Alhonen L, Janne J (2002) Targeted disruption of spermidine/spermine N¹-acetyltransferase gene in mouse embryonic stem cells. Effects on polyamine homeostasis and sensitivity to polyamine analogues. J Biol Chem 277:25323

30. Pegg A E (1988) Polyamine metabolism and its importance in neoplastic growth and a target for chemotherapy. Cancer Res 48:759

31. Porter C W, Ganis B, Libby P R, Bergeron R J (1991) Correlations between polyamine analogue-induced increases in spermidine/spermine N¹-acetyltransferase activity, polyamine pool depletion, and growth inhibition in human melanoma cell lines. Cancer Res 51:3715

32. Porter C W, Bernacki R J, Miller J, Bergeron R J (1993) Antitumor activity of N¹,N¹¹-bis(ethyl)norspermine against human melanoma xenografts and possible biochemical correlates of drug action. Cancer Res 53:581

33. Porter C W, Ganis B, Rustum Y, Wrzosek C, Kramer D L, Bergeron R J (1994) Collateral sensitivity of human melanoma multidrug-resistant variants to the polyamine analogue, N¹,N¹¹-diethylnorspermine. Cancer Res 54:5917

34. Reddy V K, Valasinas A, Sarkar A, Basu H S, Marton L J, Frydman B (1998) Conformationally restricted analogues of N¹,N¹²-bisethylspermine: synthesis and growth inhibitory effects on human tumor cell lines. J Med Chem 41:4723

35. Reddy V K, Sarkar A, Valasinas A, Marton L J, Basu H S, Frydman B (2001) Cis-unsaturated analogues of 3,8,13,18,23-pentaazapentacosane (Be-4-4-4-4): synthesis and growth inhibitory effects on human prostate cancer cell lines. J Med Chem 44:404

36. Seiler N (1995) Polyamine oxidase, properties and functions. Prog Brain Res 106:333

37. Shappell N W, Miller J T, Bergeron R J, Porter C W (1992) Differential effects of the spermine analog, N1,N12-bis(ethyl)-spermine, on polyamine metabolism and cell growth in human melanoma cell lines and melanocytes. Anticancer Res 12:1083

38. Suzuki O. Matsumoto T, Katsumata Y (1984) Determination of polyamine oxidase activities in human tissues. Experientia 40:838

39. Valasinas A, Sarkar A, Reddy V K, Marton L J, Basu H S, Frydman B (2001) Conformationally restricted analogues of N¹,N¹⁴-bisethylhomospermine (Be-4-4-4): synthesis and growth inhibitory effects on human prostate cancer cells. J Med Chem 44:390

40. Vujcic S, Diegelman P, Bacchi C J, Kramer D L, Porter C W (2002) Identification and characterization of a novel flavincontaining spermine oxidase of mammalian cell origin. Biochem J 367:665

41. Vujcic S, Liang P, Diegelman P, Kramer D, Porter C W (2003) Genomic identification and biochemical characterization of the mammalian polyamine oxidase involved in polyamine backconversion. Biochem J 370:19

42. Wang Y, Devereux W, Woster P M, Stewart T M, Hacker A, Casero R A (2001) Cloning and characterization of a human polyamine oxidase that is inducible by polyamine analogue exposure. Cancer Res 61:5370

43. Wang Y, Murray-Stewart T, Devereux W, Hacker A, Frydman B, Woster P M, Casero R A (2003) Properties of purified recombinant human polyamine oxidase, PAOh1/SMO. Biochem Biophys Res Commun 304:605

44. Webb H K, Wu Z, Sirisoma N, Ha H C, Casero R A, Woster P M (1999) 1-(N-Alkylamino)-11-(N-ethylamino)-4,8-diazaundecanes: simple synthetic polyamine analogues that differentially alter tubulin polymerization. J Med Chem 42:1415

### EXAMPLE 8

### Properties of Purified Recombinant Human Polyamine Oxidase, PAOh1/SMO

The polyamine metabolic pathway has been identified as a rational target for antineoplastic therapy [1-5]. It has been demonstrated that polyamine catabolism and the production of H₂O₂ through the oxidation of polyamines likely contribute to the cellular response to specific antitumor polyamine analogues. Consequently, interest in the potential of exploiting polyamine catabolism for therapeutic advantage has increased [6,7]. A considerable body of work has accumulated studying one rate-limiting enzyme in polyamine catabolism, spermidine/spermine N¹-acetyltransferase (SSAT) [8]. This enzyme is highly inducible by several antitumor polyamine analogues and has been linked to their cytotoxic activity [6,9-15]. A second step in polyamine catabolism is the oxidation of the acetylated polyamines by the action of a previously described FAD-dependent oxidase, polyamine oxidase (PAO), whose activity is generally limited by the availability of the acetylated substrate [16-19]. However, until very recently the role of polyamine oxidation in mammalian cells has been limited, since no verified animal polyamine oxidase had been cloned. We previously reported the cloning and preliminary characterization of a human, FAD-dependent, polyamine oxidase (PAOh1/SMO) that is highly inducible by specific antitumor polyamine analogues and can efficiently use spermine as a substrate [20]. We have also recently demonstrated using in vitro TnT produced proteins that this protein is capable of using N¹-acetylspermine as a substrate [21]. Vujcic et al. subsequently reported similar results using a mammalian cell transfection model with a cDNA construct referred to as spermine oxidase (SMO, the same sequence we originally reported as PAOh1). In these studies lysates from transfected cells demonstrated an oxidase activity that preferentially oxidized spermine [22]. However, further detailed study of human PAOh1/SMO has been limited since only in vitro TnT produced protein or mammalian transfection systems have been available. Therefore, to provide a source of readily available protein for further study, we have produced purified human recombinant PAOh1/SMO corresponding to the clone that we originally described [20] and initial characterization has been performed. Here, substrate specificity and sensitivity to inhibitors are examined. The results demonstrate that purified PAOh1/SMO codes for a protein that efficiently oxidizes spermine, less efficiently oxidizes N¹-acetylspermine, but does not use spermidine as a substrate. Additionally, a potent class of inhibitors of PAOh1/SMO is identified. The overall data support the hypothesis that PAOh1/SMO represents a new polyamine catabolic enzyme that affects polyamine homeostasis and has the potential to act as a determinant of cellular sensitivity to the antitumor polyamine analogues.

Materials and Methods

Abbreviations: PAOh1/SMO, human polyamine oxidase h1/spermine oxidase; SSAT, spermidine/spermine N¹-acetyltransferase; BEN Spm, N¹,N¹¹-bis(ethyl)norspemine; CPENSpm, N¹-ethyl-N¹¹-(cyclopropyl) methyl-4,8,diazaundecane; CHENSpm, N¹-ethyl-N¹¹-(cycloheptyl) methyl-4,8,diazaundecane; IPENSpm, (S)-N¹-(2-methyl-1-butyl)- N¹¹-ethyl-4,8,diazaundecane;MDL 72,527, (N1,N4-bis(2,3-butadienyl)-1,4-butanediamine).

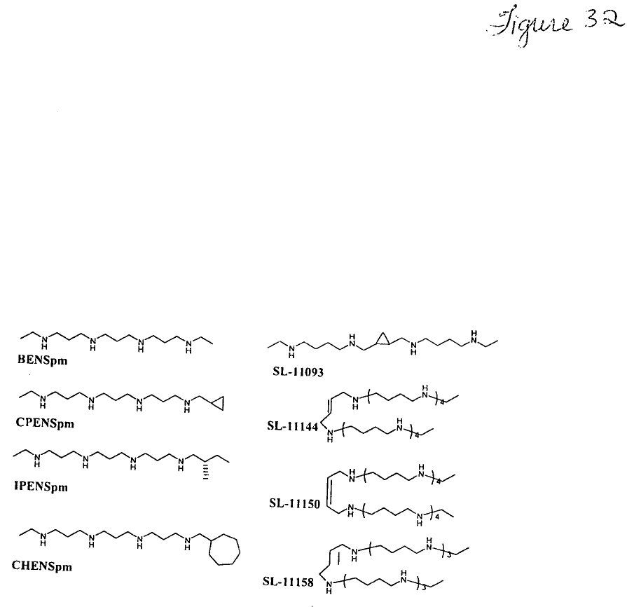

Chemicals. N¹,N¹¹-bis(ethyl)norspermine (BENSpm) was provided by Parke-Davis (Ann Arbor, Mich.). N¹-ethyl-N¹¹-(cyclopropyl)methyl-4,8, diazaundecane (CPENSpm), N¹-ethyl-N¹¹-(cycloheptyl)methyl-4,8, diazaundecane (CHENSpm), (S)-N1-(2-methyl-1-butyl)-4,8,diazaundecane (IPENSpm), SL-11093, SL-11144, SL-11150, SL-11158, and the selective PAO inhibitor N¹,N⁴-bis(2,3-butadienyl)-1,4-butanediamine (MDL 72,527) were synthesized as previously reported [23-27] (FIG. 32). Spermine, spermidine, and luminol were purchased from Sigma Chemical (St. Louis, Mo.). N1-acetylspermine was purchased from Fluka (Switzerland). Horseradish peroxidase was from Roche Molecular Biochemicals (Indianapolis, Ind.). Restriction and DNA modifying enzymes were purchased from New England Biolabs (Beverly, Mass.), Invitrogen (Carlsbad, Calif.) and Sigma. Invitrogen synthesized custom oligomers used in the experiments. Other chemicals came from Sigma, Bio-Rad (Hercules, Calif.), and J. T. Baker (Phillipsburg, N.J.).

Construction of the bacterial expression vector. A 1702 bp fragment of the PAOh1cDNA was produced by PCR using the primer pairs 50-TCGGCGCCATATGCAAAGTTGTGAATCCAGT-30 (SEQ ID NO: 31) and 50-ATTACTCGAGAGTTAGTGGCTCTTCTCAGCA-30 (SEQ ID NO: 32), and the pCR2.1/PAOh1 [20] plasmid as the template. The resultant PCR product was digested by NdeI and XhoI (underlined sequences in primers) and cloned into the His-tagged pET15b bacterial expression vector (Novagen, Madison, Wis.) in the same restriction sites, resulting in the bacterial expression vector pET15b/PAOh1/SMO.

Expression and Purification of PAOh1/SMO. The pET15b/PAOh1/SMO plasmid was used to transform the BL₂₁(DE₃) strain of Escherichia coli (Novagen) and transformed cells were selected on LB agar with 50 μg/ml ampicillin. The expression of PAOh1/SMO protein was induced in LB medium by the addition of 1 mM IPTG for 3 h at 37 ° C. Cell lysates were prepared under denaturing conditions with 8M urea and PAOh1/SMO protein was purified from the lysate by Ni-NTA resin according to the manufacturer's protocol (Qiagen, Valencia, Calif.). The resulting denatured protein was renatured in buffers containing decreasing concentrations of urea (5M urea, 4 h; 2.5M urea, 4 h; 1M urea, 12 h; and 0M urea, 12 h) and 50 mM Tris-HCl, pH 7.5, 250 mM NaCl, 0.1 mM EDTA, 1 mM DTT, and 0.2 μM flavin adenine dinucleotide (FAD).

Determination of PAO enzyme activity. PAO activity of the purified PAOh1/SMO was assayed using a modi.cation of the chemiluminesence analysis reported by Fernandez et al. [28] and Rogers, et al. [29]. Briefly, luminol-dependent chemiluminescence was determined using a Monolight 3010 luminometer with two reagent injectors. Luminol was prepared as a 100 mM stock solution in DMSO and diluted to 100 μM with H₂O, immediately prior to use. Purified PAOh1/SMO was assayed in a 100 mM glycine buffer, pH 8.0, 5 ηmol luminol, 20 μg horseradish peroxidase, and the polyamine substrate as indicated. All reagents with the exception of the polyamine substrate were combined and incubated for 2 min at 37 ° C., then the tube was transferred to the luminometer, substrate was added, and the resulting chemiluminescence was integrated over 20 s. The integral values are calibrated against standards containing known concentrations of H₂O₂ and the activities are expressed as pmols H₂O₂/mg protein/min. Where indicated, inhibitors were added at the specified concentrations prior to the addition of substrate. Kₘ and Vₘₐₓ values for the purified enzyme with the indicated substrate were estimated using Lineweaver-Burk transformation of the Michaelis-Menten kinetic equation.

Results

Expression of PAOh1/SMO in E. coli cells

High level expression of PAOh1/SMO was obtained from pET15b/PAOh1/SMO transformed BL₂₁DE₃ E. coli cells after induction with 1 mM IPTG for 3 h at 37° C., and was clearly observed as the predicted ˜64 kDa band by SDS-PAGE analysis when cell lysates were prepared under denaturing conditions (not shown). However, under non-denaturing conditions the most newly synthesized PAOh1/SMO protein was found to be insoluble and presumably located in inclusion bodies. Therefore, protein was first purified from the cell lysate under denaturing conditions, using the Ni-NTA column and renatured by dialysis against decreasing concentrations of urea. The renatured enzyme was then assayed for enzyme activity.

Enzyme Activity of Purified PAOh1/SMO

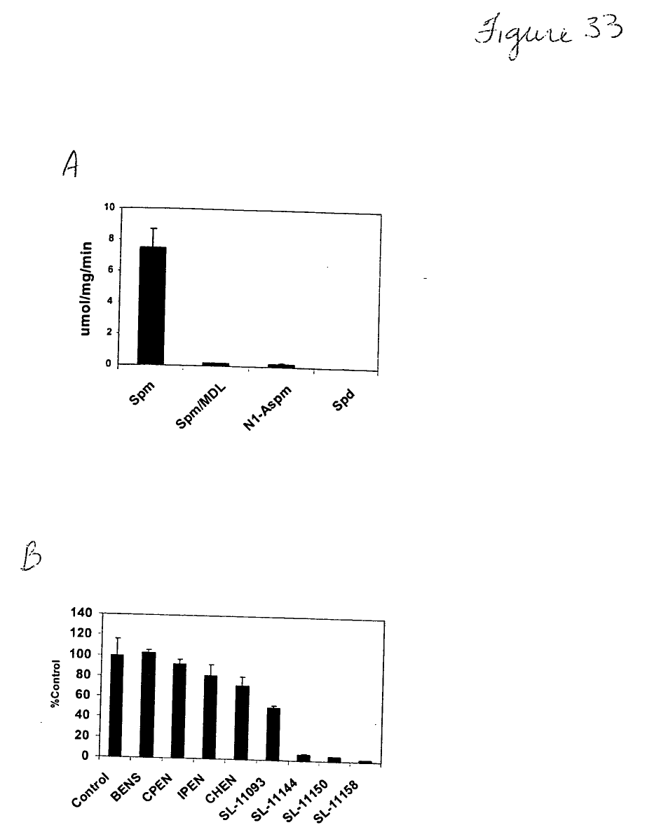

To determine the substrate specificity of PAOh1/SMO under standard reaction conditions, 250 μM spermine, spermidine, and N¹-acetylspermine were analyzed for their ability to serve as substrates (FIG. 33A), PAOh1/SMO was found to efficiently oxidize spermine. However, PAOh1/SMO was less efficient in oxidizing N¹-acetylspermine. No PAOh1/SMO activity was observed when spermidine was used as the substrate (FIG. 33A). Note that oxidation of spermine by purified PAOh1/SMO was inhibited >95% by 250 μM MDL 72,527, an inhibitor of polyamine oxidase (FIG. 3). Kₘ and Vₘₐₓ of purified PAOh1/SMO protein. To determine the apparent Kₘ and Vₘₐₓ values for the purified PAOh1/SMO protein, increasing concentrations of spermine ranging from 1 to 100 μM were used to calculate the initial velocities of substrate oxidation. The Kₘ and Vₘₐₓ values of purified PAOh1/SMO on spermine were determined by Lineweaver-Burk transformation to be 1.63 μM and 7.72 μmol/mg protein/min, respectively. However, when N¹-acetylspermine was used as substrate PAOh1/SMO demonstrated a lower affnity (Kₘ=51 μM) as well as a lower maximum velocity (Vₘₐₓ=0.251 μmol/mg protein/min).

Polyamine Analogues Do Not Serve as Substrates for PAOh1/SMO

There is evidence to suggest that some antitumor polyamine analogues are substrates for cellular oxidases, including PAO [30,31]. Therefore the ability of purified PAOh1/SMO to oxidize various antitumor polyamine analogues that are in or are being considered for clinical trials was examined. The symmetrically substituted BENSpm, and the unsymmetrically substituted CPENSpm, CHENSpm, and IPENSpm, the oligamines, SL-11144, SL-11150, and SL-11156, and the conformationally restricted analogue, SL-11093, were incubated at a concentration of 250 μM with purified PAOh1/SMO. At this concentration, none of the analogues examined were found to be oxidized by PAOh1/SMO (not shown).

Inhibition of PAOh1/SMO Activity by Polyamine Analogues

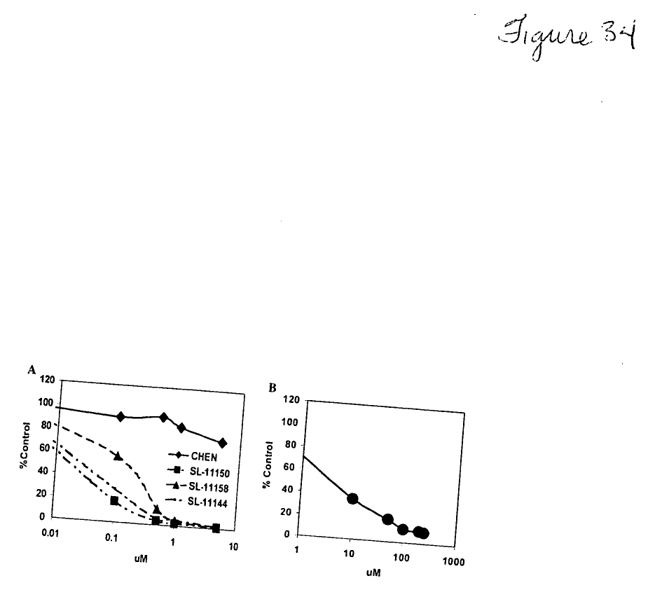

MDL 72,527 was originally designed as a specific inhibitor of PAO, the enzyme that prefers acetylated polyamines as its substrate [18,27]. However, it has clearly been demonstrated to effectively inhibit PAOh1/SMO activity at the concentration of 250 μM [19,20]. The unsymmetrically substituted polyamine analogue CHENSpm has been implicated as an inhibitor of the maize plant PAO in vitro [32]. Since oxidation of polyamines appears to be a mediator of specific analogue cytotoxicity and is significantly induced by specific antitumor polyamine analogues, the determination of which analogues act as inhibitors of PAOh1/SMO may be instructive with regard to understanding the mechanism of action of the individual analogues. Initially, to determine if any of the above analogues act as inhibitors of purified PAOh1/SMO activity (FIG. 33B), 10 μM of each was examined for its ability to inhibit the oxidation of spermine (250 μM). Of the 8 analogues examined, 3 were found to be potent inhibitors of the purified PAOh1/SMO enzyme. These 3 analogues, SL-11144, SL-11150, and SL-11158, were then compared to CHENSpm, which has been implicated in the inhibition of the maize PAO [32], and to MDL 72,527. Increasing concentrations of each inhibitor were incubated in the presence of the purified enzyme. The results of these inhibition studies (FIG. 34) clearly demonstrate that SL-11144, SL-11150, and SL-11158 are potent inhibitors of PAOh1/SMO activity, with SL-11144 and SL-11150 demonstrating an IC50<0:11M. SL-11144 and Sl-11150 were 100 times more potent than MDL 72,527 which demonstrated an IC50 of ˜10 μM. CHENSpm was not found to profoundly inhibit PAOh1/SMO activity within the range of concentrations tested.

Discussion

The modulation of polyamine catabolism has emerged as a potential target for antineoplastic intervention [6,7,14,19,20,22,33-35]. The oxidation of polyamines and their acetylated derivatives produces the diffusible reactive oxygen species, H₂O₂, whose production has been linked to the cytotoxic activity of specific antitumor polyamine analogues [6,7]. We recently cloned and preliminarily characterized a human gene that codes for the first identified mammalian polyamine oxidase, PAOh1/SMO [20]. Our initial enzymatic characterization of PAOh1/SMO was performed using protein products produced in a wheat germ linked transcription/translation (TnT) system, and demonstrated that the enzyme can efficiently catalyze the oxidation of spermine [20]. Subsequent studies using the same TnT system also indicated that PAOh1/SMO can oxidize spermidine and N¹-acetylspermine [36]. These results were in contrast to those reported by Vujcic et al. [22] who found that lysates from human cells transfected with a PAOh1/SMO expression construct could only efficiently oxidize spermine. The production of purified, recombinant PAOh1/SMO reported here has allowed us to directly address this apparent contradiction.

The purified recombinant PAOh1/SMO protein reported here has an apparent molecular weight of 64 kd. The fact that the E. coli produced protein exhibits a high specific activity indicates that extensive posttranslational modification is not required for activity. The substrate specificity exhibited by the purified protein is consistent with that observed by Vujcic et al. [19,22] and implicated by the findings of Niiranen et al. [37]. The purified protein readily uses spermine as a substrate, as well as N¹-acetylspermine. However, N¹-acetylspermine is a much poorer substrate for the enzyme than is spermine and the purified enzyme does not oxidize spermidine. The basis for the observed differences in substrate specificity as compared to our TnT produced protein is not immediately clear. A potential basis for the difference in the observations with the purified protein and the protein produced in the wheat germ TnT system may be a result of differences in protein folding or a difference in co-factors or post-translational modifications between the wheat germ system and PAOh1/SMO produced in E. coli [38]. The results with the protein produced in the wheat germ TnT system are, however, consistent with observations with the polyamine oxidases of the maize and barley plants, which are nearly identical in size to the human PAOh1/SMO protein, and possess protein domain organization very similar to the human protein. Both plant proteins are able to use both spermine and spermidine as substrates [39,40]. However, the plant enzymes do not oxidize spermine to spermidine but instead oxidize spermine to 1,3-diaminopropane, H₂O₂, and 3-(aminopropyl)-4-aminobutyraldehyde. Similarly, the plant enzymes oxidize spermidine to 1,3-diaminopropane, H₂O₂, and 4-aminobutyraldehyde [32].

It should be noted that the human PAOh1/SMO gene codes for multiple splice variants that demonstrate significant activity using spermidine, spermine, and N¹-acetylspermine as substrates when protein is produced in the wheat germ TnT system [36]. However, the PAOh1/SMO splice variant designated isoform 1 in Murray-Stewart et al. [36] and identical to the splice variant used to produce the purified protein here clearly behaves differently depending on whether it is produced in the TnT system or in the recombinant bacterial system as reported here. Therefore, it will be necessary to examine the substrate specificity of each of the isozymes coded by the various PAOh1/SMO splice variants once each is available in purified form.

At the start of these studies it was not clear if PAOh1/SMO represented a new polyamine catabolic enzyme. However, based on the results of Vujcic et al. [22] and the data produced with the purified enzymes as reported here, it is most likely that PAOh1/SMO does represent a previously unrecognized mammalian enzyme capable of oxidizing spermine. Supporting this probability is the very recent report by Porter and colleagues [19] who present convincing data defining the sequence and activity of a classical PAO that uses the N¹-acetylated polyamines as its preferred substrate [16-18]. Polyamine oxidation has been implicated in the metabolism of various antitumor polyamine analogues [30,31,41]. However, none of the analogues tested here were found to be substrates for the purified PAOh1/SMO, indicating that this isoform is not responsible for the metabolism of these representative analogues. These results are entirely consistent with those of Vujcic et al. [19] using similar compounds.

Interestingly, three of the analogues examined were determined to be potent inhibitors of PAOh1/SMO. SL-11144, SL-111150, and SL-11158 each was capable of inhibiting PAOh1/SMO>90% at concentrations less than 1 μM. Additionally, each of these compounds was significantly more potent than MDL 72, 527 that was originally synthesized as a specific PAO inhibitor [27]. CHENSpm has been reported to be an inhibitor of the maize PAO [32]. However, it appears to be a poor inhibitor of the human enzyme, only inhibiting PAOh1/SMO activity <20% at 10 μM. The discovery of potent inhibitors of PAOh1/SMO should be helpful in dissecting the role of its oxidase activity in both polyamine homeostasis and in determining the sensitivity to various polyamine analogues.

Since one of the products of polyamine catabolism is H₂O₂, the potential for a tumor-specific drug-induced increase in polyamine catabolism is an intriguing possibility that is actively being pursued as an antineoplastic strategy. Until recently, polyamine catabolism was thought to be solely under the control of the rate-limiting activity of SSAT. However, the newest data clearly indicate that this pathway is much more complex than that originally hypothesized. We and others have demonstrated that the PAOh1/SMO activity is inducible by specific antitumor polyamine analogues [19,20,42]. This increase in activity and subsequent production of H₂O₂ may play an active role in determining cellular sensitivity to these agents as it has been suggested [6,7]. Porter and colleagues have recently presented data indicating that the classical PAO is also an inducible enzyme in specific instances, further implicating a complex regulation of catabolism [19]. The availability of a purified PAOh1/SMO protein will now allow the detailed study of an enzyme whose activity may be critical to both polyamine homeostasis in normal cells and an important determinant of tumor cell response to agents that alter polyamine metabolism.

References

[1] T. Thomas, T. J. Thomas, Polyamines in cell growth and cell death: molecular mechanisms and therapeutic applications, Cell. Mol. Life Sci. 58 (2001) 244-258.

[2] R. G. Schipper, G. Deli, P. Deloyer, W. P. Lange, J. A. Schalken, A. A. Verhofstad, Antitumor activity of the polyamine analog N¹,N¹¹-diethylnorspermine against human prostate carcinoma cells, Prostate 44 (2000) 313-321.

[3] L. J. Marton, A. E. Pegg, Polyamines as targets for therapeutic intervention, Annu. Rev. Pharmacol. Toxicol. 35 (1995) 55-91.

[4] R. A. Casero, P. M. Woster, Terminally alkylated polyamine analogues as chemotherapeutic agents, J. Med. Chem. 44 (2001) 1-26.

[5] N. E. Davidson, H. A. Hahm, D. E. McCloskey, P. M. Woster, R. A. Casero Jr., Clinical aspects of cell death in breast cancer: the polyamine pathway as a new target for treatment, Endocr. Relat. Cancer 6 (1999) 69-73.

[6] H. C. Ha, P. M. Woster, J. D. Yager, R. A. Casero Jr., The role of polyamine catabolism in polyamine analogue-induced programmed cell death, Proc. Natl. Acad. Sci. USA 94 (1997) 11557-11562.

[7] Y. Chen, D. L. Kramer, P. Diegelman, S. Vujcic, C. W. Porter, Apoptotic signaling in polyamine analogue-treated SK-MEL-28 human melanoma cells, Cancer Res. 61 (2001) 6437-6444.

[8] R. A. Casero Jr., A. E. Pegg, Spermidine/spermine N1-acetyltransferase—the turning point in polyamine metabolism, FASEB J. 7 (1993) 653-661.

[9] R. A. Casero Jr., P. Celano, S. J. Ervin, C. W. Porter, R. J. Bergeron, P. R. Libby, Differential induction of spermidine/spermine N¹-acetyltransferase in human lung cancer cells by the bis(ethyl)polyamine analogues, Cancer Res. 49 (1989) 3829-3833.

[10] R. A. Casero Jr., A. R. Mank, L. Xiao, J. Smith, R. J. Bergeron, P. Celano, Steady-state messenger RNA and activity correlates with sensitivity to N¹,N¹²-bis(ethyl)spermine in human cell lines representing the major forms of lung cancer, Cancer Res. 52 (1992) 5359-5363.

[11] E. W. Gabrielson, A. E. Pegg, R. A. Casero Jr., The induction of spermidine/spermine N¹-acetyltransferase (SSAT) is a common event in the response of human primary non-small cell lung carcinomas to exposure to the new antitumor polyamine analogue N¹,N¹¹-bis(ethyl)norspermine, Clin. Cancer Res. (1999) 1638-1641.

[12] M. Fogel-Petrovic, S. Vujcic, P. J. Brown, M. K. Haddox, C. W. Porter, Effects of polyamines, polyamine analogs, inhibitors of protein synthesis on spermidine-spermine N¹-acetyltransferase gene expression, Biochemistry 35 (1996) 14436-14444.

[13] M. Fogel-Petrovic, D. L. Kramer, S. Vujcic, J. Miller, J. S. McManis, R. J. Bergeron, C. W. Porter, Structural basis for differential induction of spermidine/spermine N¹-acetyltransferase activity by novel spermine analogs, Mol. Pharmacol. 52 (1997) 69-74.

[14] D. E. McCloskey, A. E. Pegg, Altered spermidine/spermine N¹-acetyltransferase activity as a mechanism of cellular resistance to bis(ethyl)polyamine analogues, J. Biol. Chem. 275 (2000) 28708-28714.

[15] S. Vujcic, M. Halmekyto, P. Diegelman, G. Gan, D. L. Kramer, J. Janne, C. W. Porter, Effects of conditional overexpression of spermidine/spermine N1-acetyltransferase on polyamine pool dynamics, cell growth, sensitivity to polyamine analogs, J. Biol. Chem. 275 (2000) 38319-38328.

[16] E. Holtta, Oxidation of spermidine and spermine in rat liver: purification and properties of polyamine oxidase, Biochemistry 16 (1977) 91-100.

[17] E. Holtta, Polyamine oxidase (rat liver), Methods Enzymol. 94 (1983) 306-311.

[18] N. Seiler, Polyamine oxidase, properties and functions, Prog. Brain Res. 106 (1995) 333-344.

[19] S. Vujcic, P. Liang, P. Diegelman, D. Kramer, C. W. Porter, Genomic identification and biochemical characterization of the mammalian polyamine oxidase involved in polyamine backconversion. Biochem. J. 370 (2003) 19-28.

[20] Y. Wang, W. Devereux, P. M. Woster, T. M. Stewart, A. Hacker, R. A. Casero Jr., Cloning and characterization of a human polyamine oxidase that is inducible by polyamine analogue exposure, Cancer Res. 61 (2001) 5370-5373.

[21] T. Murray-Stewart, Y. Wang, W. Devereux, R. A. Casero, Cloning and characterization of human polyamine oxidase splice variants, Proc. Am. Assoc. Cancer Res. 43 (2002) 1120-1121.

[22] S. Vujcic, P. Diegelman, C. J. Bacchi, D. L. Kramer, C. W. Porter, Identification and characterization of a novel flavin-containing spermine oxidase of mammalian cell origin, Biochem. J. 367 (2002) 665-675.

[23] H. K. Webb, Z. Wu, N. Sirisoma, H. C. Ha, R. A. Casero Jr., P. M. Woster, 1-(N-alkylamino)-11-(N-ethylamino)-4,8-diazaundecanes: simple synthetic polyamine analogues that differentially alter tubulin polymerization, J. Med. Chem. 42 (1999) 1415-1421.

[24] V. K. Reddy, A. Valasinas, A. Sarkar, H. S. Basu, L. J. Marton, B. Frydman, Conformationally restricted analogues of N1,N12-bisethylspermine: synthesis and growth inhibitory effects on human tumor cell lines, J. Med. Chem. 41 (1998) 4723-4732.

[25] V. K. Reddy, A. Sarkar, A. Valasinas, L. J. Marton, H. S. Basu, B. Frydman, cis-Unsaturated analogues of 3,8,13,18,23-pentaazapentacosane (BE-4-4-4-4): synthesis and growth inhibitory effects on human prostate cancer cell lines, J. Med. Chem. 44 (2001) 404-417.

[26] A. Valasinas, A. Sarkar, V. K. Reddy, L. J. Marton, H. S. Basu, B. Frydman, Conformationally restricted analogues of N1,N14-bisethylhomospermine (be-4-4-4): synthesis and growth inhibitory effects on human prostate cancer cells, J. Med. Chem. 44 (2001) 390-403.

[27] P. Bey, F. N. Bolkenius, N. Seiler, P. Casara, N-2,3-butadienyl-1,4 derivatives: potent irreversible inactivators of mammalian polyamine oxidase, J. Med. Chem. 28 (1985) 1-2.

[28] C. Fernandez, R. M. Sharrard, M. Talbot, B. D. Reed, N. Monks, Evaluation of the significance of polyamines and their oxidases in the aetiology of human cervical carcinoma, Br. J. Cancer 72 (1995) 1194-1199.

[29] M. S. Rogers, S. F. Yim, K. C. Li, C. C. Wang, M. Arumanayagam, Cervical intraepithelial neoplasia is associated with increased polyamine oxidase and diamine oxidase concentrations in cervical mucus, Gynecol. Oncol. 84 (2002) 383-387.

[30] N. Seiler, B. Duranton, F. Vincent, F. Gosse, J. Renault, F, Raul, Inhibition of polyamine oxidase enhances the cytotoxicity of polyamine oxidase substrates. A model study with N1-(N-octanesulfonyl) spermine and human colon cancer cells, Int. J. Biochem. Cell. Biol. 32 (2000)703-716.

[31] K. R. Lawson, S. Marek, J. A. Linehan, P. M. Woster, R. A. Casero Jr., C. M. Payne, E. W. Gerner, Detoxi.cation of the polyamine analogue N1-ethyl-N11-[(cycloheptyl)methy]-4,8-diazaundecane (chenspm) by polyamine oxidase, Clin. Cancer Res. 8 (2002) 1241-1247.

[32] C. Binda, R. Angelini, R. Federico, P. Ascenzi, A. Mattevi, Structural bases for inhibitor binding and catalysis in polyamine oxidase, Biochemistry 40 (2001) 2766-2776.

[33] S. Lamond, H. M. Wallace, Polyamine oxidase activity and growth in human cancer cells, Biochem. Soc. Trans. 22 (1994) 396S.

[34] H. M. Wallace, J. Duthie, D. M. Evans, S. Lamond, K. M. Nicoll, S. D. Heys, Alterations in polyamine catabolic enzymes in human breast cancer tissue, Clin. Cancer Res. 6 (2000) 3657-3661.

[35] R. A. Casero, Y. Wang, T. M. Stewart, W. Devereux, A. Hacker, R. Smith, P. M. Woster, The role of polyamine catabolism in antitumor drug response, Biochem. Soc. Trans. 31 (2003) 361-365.

[36] T. Murray-Stewart, Y. Wang, W. Devereux, R. A. Casero Jr., Cloning and characterization of multiple human polyamine oxidase splice variants that code for isoenzymes with different biochemical characteristics, Biochem. J. 368 (2002) 673-677.

[37] K. Niiranen, M. Pietila, T. J. Pirttila, A. Jarvinen, M. Halmekyto, V. P. Korhonen, T. A. Keinanen, L. Alhonen, J. Janne, Targeted disruption of spermidine/spermine N¹-acetyltransferase gene in mouse embryonic stem cells. E.ects on polyamine homeostasis and sensitivity to polyamine analogues, J. Biol. Chem. 277 (2002) 25323-25328.

[38] F. U. Hartl, M. Hayer-Hartl, Molecular chaperones in the cytosol: from nascent chain to folded protein, Science 295 (2002) 1852-1858.

[39] R. Federico, C. Alisi, A. Cona, R. Angelini, Puri.cation of polyamine oxidase from maize seedlings by immunoadsorbent column, Adv. Exp. Med. Biol. 250 (1988) 617-623.

[40] R. Federico, A. Cona, R. Angelini, M. E. Schinina, A. Giartosio, Characterization of maize polyamine oxidase, Phytochemistry 29 (1990) 2411-2414.

[41] N. Seiler, L. Badolo, B. Duranton, F. Vincent, Y. Schneider, F. Gosse, F. Raul, E.ect of the polyamine oxidase inactivator mdl 72527 on N1-(N-octanesulfonyl)spermine toxicity, Int. J. Biochem. Cell. Biol. 32 (2000) 1055-1068.

[42] W. Devereux, Y. Wang, T. Murray-Stewart, A. Hacker, R. A. Casero, Differential induction of polyamine oxidase by polyamine analogues in various human lung carcinoma lines, Proc. Am. Assoc. Cancer Res. 43 (2002) 963-964.

### EXAMPLE 9

### Susceptibility to Antitumor Polyamine Analogues

Recent data indicate that the expression of human PAOh1/SMO polyamine oxidase is functionally associated with the cytotoxic response of tumor cells to specific antitumor polyamine analogues that induce its expression. Specifically, the production of H₂O₂ and 3-aminopropanal by PAOh1/SMO are cytotoxic when produced acutely in high amounts as is the case when PAOh1/SMO activity is induced by the specific analogues. Therefore, the detection of the expression of the oxidase (or splice variants thereof) before and/or after exposure to an antitumor polyamine analogue can be of significant prognostic and diagnostic value. For example, cells from a tumor or a tumor type (e.g. prostate cancer cells) are tested in vitro to see if they respond to treatment with a specific antitumor polyamine analogue by producing PAOh1/SMO polyamine oxidase (or a splice variant thereof). If PAOh1/SMO is detected at greater than normal levels, then it is predicted that the tumor or tumor type will respond favorably to treatment with the analog, i.e. the treatment will be effective in killing the tumor cells and is thus appropriate. Conversely, if induction of PAOh1/SMO is not detected, then an alternative analog or treatment should be considered. Alternatively, tumor cells may be sampled before and/or after treatment with an analog to ascertain the extent of induction of PAOh1/SMO, for the purpose of, for example, monitoring the course of treatment, or optimiziing the dose of analog, etc.

Such detection may be carried out, for example, by detecting the oxidase protein directly (e.g. with antibodies, or through measurement of oxidase activity, etc.) or by detecting the associated mRNA (e.g. via real time PCR, standard PCR, Northern analysis, or RNAse protection, etc.).

Antibody: Based on the use of RNA and protein synthesis inhibitors, the induction of PAOh1/SMO activity in response to analogue exposure appears to be the result of new mRNA synthesis followed by newly synthesized protein. Additionally, there is no evidence of significant post translational regulation of PAOh1/SMO protein. Consequently there is an apparent direct correlation between protein amount and enzyme activity. This result is identical to that observed with another polyamine catabolic enzyme, spermidine/spermine N¹-acetyltransferase (SSAT). Therefore, as has been previously demonstrated for SSAT, the development of a specific antisera that recognizes PAOh1/SMO protein will be an invaluable tool for the prognostic and diagnostic evaluation of tumor response to the antitumor polyamine analogues. Toward this end we have proceeded to develop specific antisera to PAOh1/SMO. It should be noted that the recombinant human PAOh1/SMO protein is not an effective immunogenic protein in rabbits. Therefore we have developed peptides based on the Kyte-Doolittle method for calculating hydrophilicity. The sequences chosen (H₂N-EEPRGGRWDEDEQ-COOH [SEQ ID NO: 31] and H₂N-EEVRNRIRNDPDD-COOH [SEQ ID NO:32]) are predicted to have the greatest immunogenicity based on there hydrophilic character. Initial immunoprecipitation testing of the antisera from immunized rabbits indicates that the antisera are capable of recognizing the recombinant human PAOh1/SMO protein. Analogue treated tissues and cells may be analyzed by, for example, immunohistochemical and Western analysis using such anti-PAOh1/SMO antibodies.

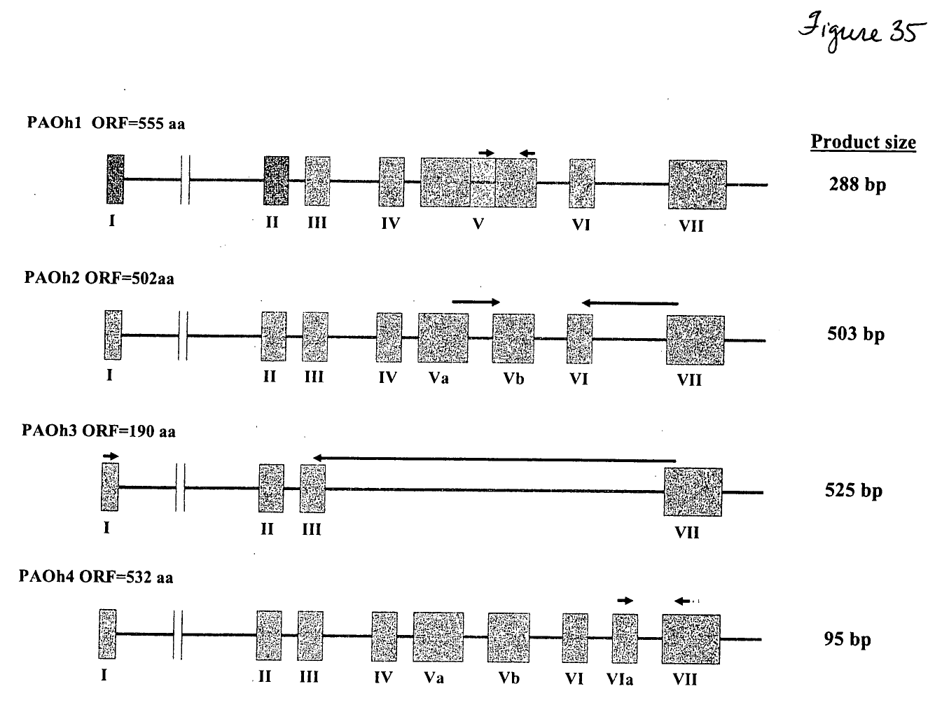

Real Time PCR: Since the current data indicate that the major level of regulation of PAOh1/SMO induction occurs at the level of increased mRNA, the potential exists to measure tissue response to antitumor polyamine analogue exposure by measuring the amount of PAOh1/SMO mRNA induced. One of the most sensitive and quantifiable methods available is real time PCR. Primers have been developed that can be used to quantitatively identify all four of the major human PAOh1 gene splice variants (Table 2 and FIGS. 35 and 36). The ability to individually quantify the expression of each splice variant may be important in the event that particular splice variants are linked either to disease etiology or drug response.

The methods of the present invention may thus be used to detect expression of PAOh1/SMO oxidase. In order to be considered significant, the amount of PAOh1/SMO oxidase expression that is detected will be at least in the range of from about 1.5 to about 20 or more times greater (or lower) than basal expression of the enzyme, or of basal expression of the splice variant. By “basal expression of the enzyme” we mean the level of expression typically detected in healthy, disease-free individuals. Those of skill in the art are familiar with the establishment of such base-line levels of expression of a biological product in order to provide a standard for comparison for diagnostic/prognostic purposes.

TABLE 2
Real time PCR primers for human PAO/SMO

| | | |
|---|---|---|
| PAOh1 | | |
| RT1-for: | 5′ GAT CCC GGC GGA CCA TGT GAT TGT G 3′ | (SEQ ID NO: 33) |
| P203: | 5′ CTC AGG CGG GTA GAG GAC ATC AAA 3′ | (SEQ ID NO: 34) |
| PAOh2 | | |
| RT2-for: | 5′ GCC CCG GGG TGT GCT AAA GAG 3′ | (SEQ ID NO: 35) |
| RT2b-rev: | 5′ CCT GCA TGG GCG CTG TCT TTG 3′ | (SEQ ID NO: 36) |
| PAOh3: | | |
| RT3-for: | 5′ CGC AGS CTT ACT TCC CCG GCT CAG 3′ | (SEQ ID NO: 37) |
| RT3-rev: | 5′ CTG CAT GGG CTC GTT GTA TAA ATC 3′ | (SEQ ID NO: 38) |
| POOh4: | | |
| RT4-for: | 5′ GGA TGC TAA CAG GGG CGC CGT AAA 3′ | (SEQ ID NO: 39) |
| RT4-rev: | 5′ GCA GAG CAC CGT GGG TGG TGG AAT A 3′ | (SEQ ID NO: 40) |

### EXAMPLE 10

### Transgenic Mice

Emerging data indicate that oxidative stress and etiology of specific cancers, including prostate cancer, are closely linked. The prostate represents the human tissue with the highest intracellular concentration of spermine, the preferred substrate for PAOh1/SMO. If there were inappropriate expression of this enzyme over the lifetime of an individual, the resultant exposure to the products of PAOh1/SMO activity, H₂O₂ and 3-aminopropanal, might be expected to produce genotypic changes known to be associated with the etiology of cancer. Currently, the only mouse model for spontaneous prostate cancer is the TRAMP model that uses the over expression of SV40 large T-antigen under the control of the prostate specific probasin (androgen regulated) promoter. Although this model has been extremely useful and instructive, the molecular mechanisms leading to the disease in this transgenic model does not in any way resemble the actually etiology of human prostate cancer. In an attempt to remedy this, we have created transgenic mice that express PAOh1/SMO under control of the same prostate-specific probasin promoter. Mice expressing high levels of PAOh1/SMO may undergo preneoplastic and neoplastic changes that are more typical of the changes that occur in the development of human disease. During the 2 year life span of the mice, they may develop prostatic intraepithelial neoplasia (PIN). This model will be much more useful and instructive that the currently available TRAMP mouse model, both with respect to understanding the origins of human prostate cancer and as a model to test chemopreventive strategies (see immediately below).

The present invention thus encompasses a transgenic non-human animal that expresses PAOh1/SMO either inducibly or constitutively. In a preferred embodiment of the present invention, the transgenic animal is a mouse. The techniques used in the development of transgenic animals such as mice are well known to those of skill in the art. There are a number of methods to introduce the exogenous DNA into the germ line of an animal. One method is by microinjection of the gene construct into the pronucleus of an early stage embryo (e.g., before the four-cell stage) A detailed procedure to produce such transgenic mice has been described (see, for example, U.S. Pat. No. 5,175,383 to Leder et al., 1992, the complete contents of which are hereby incorporated by reference). This procedure has also been adapted for other mammalian species. Another method for producing germ-line transgenic mammals is through the use of embryonic stem cells. The gene construct may be introduced into embryonic stem cells by homologous recombination in a transcriptionally active region of the genome. A suitable construct may also be introduced into the embryonic stem cells by DNA-mediated transfection, such as electroporation. Detailed procedures for culturing embryonic stem cells and the methods of making transgenic mammals from embryonic stem cells can be found in Teratocarcinomas and Embryonic Stem Cells, A practical Approach, ed. E. J. Robertson (IRL Press, 1987).

In one embodiment of the invention, PAOh1/SMO expression is under control of the prostate-specific probasin promoter. However, those of skill in the art will recognize that it is possible to have expression of PAOh1/SMO under control of other promoters, for example the keratin 5/6 promoters for skin-specific expression, or the MMTV promoter for breast. Further, other elements related to expression of PAOh1/SMO may also be included, such as enhancer elements and the like. Further, the form of PAOh1/SMO that is expressed may be full-length enzyme, or a splice variant thereof.

Chemoprevention

As described above, there is ample evidence that oxidative stress has a role in the etiology of prostate cancer. Therefore, the above transgenic model may successfully mimick the earliest stages of prostate cancer, and be an ideal model for the testing of chemopreventative strategies including small molecule therapies. This would be particularly true of agents that act as specific inhibitors of the PAOh1/SMO oxidase. The transgenic model may become the standard model in which chemopreventative agents targeting prostate cancer will be tested.

### EXAMPLE 11

### Therapy Example

Although the chronic production of the reactive oxygen species (ROS) can lead to genotoxic changes and potentially carcinogenic transformation, the acute, targeted production of ROS like H₂O₂ can also lead to apoptotic cell death. Since the high induction of PAOh1/SMO activity by specific antitumor polyamine analogues appears to be a tumor type-specific event and not a general phenomenon, the acute induction of PAOh1/SMO activity may be used for therapeutic advantage. Specifically, by treating cancers with agents that selectively and rapidly induce high PAOh1/SMO activity within the tumor, it may be possible to stimulate apoptotic death in the tumor cells through the production of ROS. It has been demonstrated that the induction of polyamine catabolism and the subsequent production of H₂O₂ were directly associated with the apoptotic response of human non-small cell lung cancer cells. However, this finding occurred prior to the discovery of the PAOh1/SMO enzyme and was thought to be the result of another enzyme in the polyamine catabolic pathway. Regardless, those experiments demonstrated the proof-of-principle that rapidly induced ROS production specifically in tumor cells can induce apoptosis. We anticipate as the regulation of PAOh1/SMO in response to polyamine analogue exposure is better understood it will be possible to identify the most effective and selective agents to kill tumor cells through the induction of PAOh1/SMO. This has already been demonstrated in lung cancers and it is likely that it will be successful in prostate, breast and other important solid tumors as well. The induction of PAOh1/SMO in the cancer cells may be carried out by any of several means that are known to those of skill in the art, including but not limited to exposing the cancer cells to an antitumor polyamine analog, through the introduction of PAOh1/SMO encoding DNA or mRNA into the cancer cells (e.g. by gene therapy), or by combinations of these techniques. It will be understood by those of skill in the art that, whereas some cancer cells treated in this matter will be killed outright, others may only be damaged or weakened, or may lose their ability to proliferate, etc. Thus, this method may be used alone, or in conjunction with other cancer treatments, e.g. chemotherapy, radiation, etc. The method may be used to increase the efficacy of the killing of cancer cells by these other methods.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A substantially purified polynucleotide isolated from a mammal and encoding a polypeptide with polyamine oxidase activity.

2. The substantially purified polynucleotide of claim 1, wherein said mammal is a human.

3. The substantially purified polynucleotide of claim 1, wherein said substantially purified polynucleotide encodes an isoform or truncation of mammalian PAO.

4. The substantially purified polynucleotide of claim 1, wherein said substantially purified polynucleotide is DNA.

5. The substantially purified polynucleotide of claim 1, wherein said substantially purified polynucleotide is RNA.

6. The substantially purified polynucleotide of claim 4, wherein a nucleotide sequence of said DNA is selected from the group consisting of SEQ ID NO. 1, SEQ ID NO.3, SEQ ID NO.5, SEQ ID NO.7, SEQ ID NO.9, SEQ ID NO.11, SEQ ID NO. 13, and SEQ ID NO. 15.

7. Substantially purified polyamine oxidase isolated from a mammal.

8. The substantially purified polyamine oxidase of claim 7, wherein said mammal is a human.

9. The substantially purified polyamine oxidase of claim 7, wherein said polyamine oxidase is an isoform or truncation of PAO.

10. The substantially purified polyamine oxidase of claim 7, wherein an amino acid sequence of said polyamine oxidase is selected from the group consisting of SEQ ID NO. 2, SEQ ID NO.4, SEQ ID NO.6, SEQ ID NO.8, SEQ ID NO.10, SEQ ID NO.12, SEQ ID NO. 14, and SEQ ID NO. 16.

11. A vector comprising,
a substantially purified polynucleotide encoding a polypeptide with polyamine oxidase activity isolated from a mammal, or fragment thereof.

12. The vector of claim 11 wherein said mammal is a human.

13. The vector of claim 11 wherein said isolated polynucleotide encodes an isoform or truncation of mammalian PAO.

14. The vector of claim 11 wherein a nucleotide sequence of said isolated polynucleotide is selected from the group consisting of SEQ ID NO. 1, SEQ ID NO.3, SEQ ID NO.5, SEQ ID NO.7, SEQ ID NO.9, SEQ ID NO.11, SEQ ID NO. 13, and SEQ ID NO. 15and fragments thereof.

15. The vector of claim 11, said isolated polynucleotide encodes a polypeptide with a sequence selected from the group consisting of SEQ ID NO. 2, SEQ ID NO.4, SEQ ID NO.6, SEQ ID NO.8, SEQ ID NO.10, SEQ ID NO.12 SEQ ID NO. 14, and SEQ I) NO. 16, and fragments thereof.

16. A method for detecting PAO-related DNA or RNA in a cell, comprising,
probing said cell with a probe, said probe comprising,
a substantially purified polynucleotide encoding a polypeptide with polyamine oxidase activity isolated from a mammal, or isoform, truncation, or fragment thereof.

17. A host, comprising,
a vector comprising a substantially purified polynucleotide encoding a polypeptide with polyamine oxidase activity isolated from a mammal, or isoform, truncation, or fragment thereof.

18. The host of claim 17, wherein said host is selected from the group consisting of bacteria, yeast, mammalian cells, and insect cells.

19. An antibody to substantially purified polyamine oxidase isolated from a mammal, or to an isoform, truncation, or fragment thereof.

20. The antibody of claim 19, wherein said antibody is polyclonal.

21. The antibody of claim 19, wherein said antibody is monoclonal

22. A diagnostic or prognostic method for evaluating a response of a tumor to an antitumor polyamine analog, comprising the step of
detecting expression of PAOh1/SMO oxidase or a splice variant thereof in said tumor, wherein detection of said expression indicates that said tumor is responding or will respond favorably to treatment with said antitumor polyamine analog.

23. The method of claim 22, wherein said method is carried out prior to treatment of said tumor with said antitumor polyamine analog.

24. The method of claim 22, wherein said method is carried out after treating said tumor with said antitumor polyamine analog.

25. The method of claim 22, wherein said step of detecting is carried out using an antibody to detect PAOh1/SMO oxidase or a splice variant thereof.

26. The method of claim 22, wherein said step of detecting is carried out via real time PCR amplification to detect mRNA encoding PAOh1/SMO oxidase or a splice variant thereof.

27. A method for diagnosing a predisposition to cancer in a patient, comprising the step of
detecting expression of PAOh1/SMO oxidase or a splice variant thereof in cells of said patient, wherein detection of said expression is associated with a predisposition to develop cancer in said cells.

28. The method of claim 27, wherein said cells are selected from the group consisting of prostate cells, lung cells, and breast cells.

29. The method of claim 27, wherein said step of detecting is carried out using antibodies to detect PAOh1/SMO oxidase or a splice variant thereof.

30. The method of claim 27, wherein said step of detecting is carried out via real time PCR amplification to detect mRNA of PAOh1/SMO oxidase or a splice variant thereof.

31. A method of killing cancer cells, comprising the step of
inducing the production of PAOh1/SMO oxidase or a splice variant thereof in said cancer cells, wherein said step of inducing causes apoptotic cell death of said cancer cells.

32. The method of claim 31, wherein said cancer cells are solid tumor cells.

33. The method of claim 32, wherein said solid tumor cells are selected from the group consisting of lung, prostate, and breast tumor cells.

34. A transgenic mouse, wherein said transgenic mouse expresses PAOh1/SMO oxidase or a splice variant thereof.

35. The transgenic mouse of claim 34, wherein expression of PAOh1/SMO oxidase is under control of the probasin promoter.
